(19) United States
(12) Patent Application Publication
(10) Pub. No.: US 2024/0108629 A1
(43) Pub. Date: Apr. 4, 2024

(54) AZAQUINAZOLINE DERIVATIVES FOR USE IN TREATING OR PREVENTING DIROFILARIA INFECTION IN A MAMMAL

(71) Applicants: Liverpool School of Tropical Medicine, Liverpool (GB); The University of Liverpool Foundation Building, Liverpool (GB)

(72) Inventors: Joseph D. Turner, Liverpool (GB); Weiquan David Hong, Liverpool (GB); Paul M. O'Neill, Liverpool (GB); Stephen A. Ward, Liverpool (GB); Mark J. Taylor, Liverpool (GB)

(21) Appl. No.: 18/272,697

(22) PCT Filed: Jan. 17, 2022
(86) PCT No.: PCT/EP22/50922

§ 371 Date: Jul. 17, 2023

(30) Foreign Application Priority Data
Jan. 18, 2021 (GB) 2100635.8

(51) Int. Cl. A61K 31/5377; A61K 31/519; A61P 33/10

(52) U.S. Cl. CPC A61K 31/5377; A61K 31/519; A61P 33/10

(57) ABSTRACT

Provided is a compound of formula I, or pharmaceutically acceptable salt or solvate thereof, as defined herein for use in the treatment or prevention of a Dirofilaria infection in a mammal,

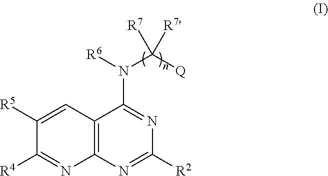

The compounds are also for use in the treatment or prevention of diseases or conditions caused by a Dirofilaria infection in a mammal. Also described are corresponding methods of treating or preventing a Dirofilaria infection in a mammal.

### INTRODUCTION

The present disclosure relates to compounds for use in the treatment or prevention of Dirofilaria infections in mammals, as well as methods for the treatment and prevention of Dirofilaria infection in mammals. The compounds and methods defined herein also find application in the treatment or prevention of a condition or a disorder caused by Dirofilaria infection in mammals, such as canine heartworm disorder or a heartworm associated respiratory disease.

### BACKGROUND OF THE INVENTION

Mammals are often susceptible to parasite infections with endoparasites such as filariae. Filariae are vector-borne, tissue-dwelling parasitic worms that cause tropical diseases, such as lymphatic filariasis, onchocerciasis, loiasis, and mansonellosis in humans (Taylor et al., 2010), and dirofilariasis in cats and dogs (Simon et al., 2012). Dirofilaria spp. can also cause zoonotic infections in humans (McCall et al., 2008; Simon et al., 2012).

One type of dirofilarial parasite which seriously harms mammals is Dirofilaria immitis (D. immitis) also known as heartworm. D. immitis is a major veterinary filarial pathogen causing chronic heartworm disease (HWD) and occasional death in dogs, cats and ferrets. Pathology develops following the establishment of adult nematodes in the right chambers of the heart following a generally asymptomatic subcutaneous and muscle migratory larval infection phase.

D. immitis go through several life stages before they become adults infecting the host mammal. The worms require the mosquito as an intermediate host to complete their life cycles. The period between the initial infection when the definitive host (e.g. dog) is bitten by a mosquito and the maturation of the worms into adults living in the heart takes six to seven months in dogs and is known as the “prepatent period”. L3 larvae migrate during blood feeding of the mosquito to the tip of the mosquito's mouth parts (labium), leave the mosquito and are deposited on the skin of the dog where they then migrate through the bite wound into the host. Most L3 larvae molt to fourth-stage larvae (L4s) in canine subcutaneous tissues within 1-3 days after infection. They then migrate to the muscles of the chest and abdomen, and 45 to 60 days after infection, molt to the fifth stage (L5, immature adult). Between 75 and 120 days after infection, these immature heartworms then enter the bloodstream and are carried through the heart to reside in the pulmonary artery. Around seven months after infection, D. immitis adults reach maturity and sexually reproduce in the pulmonary arteries and right ventricle. Adult males are around 15 cm in length, and females are around 25 cm in length and their normal life span as adults is calculated to be about 5 years. After mating, female worms release larvae known as microfilariae (mf) into the circulation. The microfilariae circulate in the bloodstream for as long as two years, waiting for the next stage in their life cycles in the gut of a bloodsucking mosquito. When ingested by a mosquito, the microfilariae undergo a series of molts to the infective third larval stage, and then migrate to the salivary glands of the mosquito, where they wait to infect another host.

Canine heartworm disease (HWD) is a severe and life-threatening disease. Pathology is generally chronic-progressive, associated with enlargement and hyper-proliferation of endocardium and physical blockage of adult worms in the pulmonary artery contributing to vessel narrowing, hypertension and ultimately heart failure (Simon et al., 2012).

D. immitis causes a more acute immunopathology in cats, due to the less well adapted nature of the parasite-host relationship. In cats, arrival of immature worms often triggers an overt eosinophilic inflammatory reaction in the lungs leading to heartworm associated respiratory disease (HARD) (McCall et al., 2008). Both cats and dogs are at risk of acute, fatal thromboembolisms when dead adult worms lodge in pulmonary vasculature (Simon et al., 2012). D. immitis can also cause kidney damage attributable to immune-complex deposition progressing from chronic infections and are likely attributable to the large antigenic burden of adults and circulating mf (Simon et al., 2012), which can attain numbers of more than 100,000/ml peripheral blood.

The related Dirofilaria, D. repens, develops exclusively in the subcutaneous tissues (Genchi and Kramer, 2017). Whilst it does not provoke the life-threatening cardiopulmonary pathologies of HWD/HARD, it can cause a range of dermatological symptoms.

D. immitis and D. repens have a widespread distribution throughout the tropics and sub-tropics although D. repens is not endemic to The Americas (Genchi and Kramer, 2017). Within the USA there is generally good knowledge of the disease and access to molecular diagnostics and thus it is the most extensively monitored country for HWD. Recent survey data indicates HWD is endemic in all US states with a State prevalence between 1-12% (Lee et al., 2010), equating to roughly one out of ten pet dogs being infected (AHS, 2016). However, in ‘hot-spot’ transmission areas of the Southern Gulf States regional prevalence above 50% has been reported (Bowman et al., 2009). In 2012, 48,000 dogs tested positive for heartworm in the USA and in 2016 over one million pets were estimated to carry disease (AHS, 2016). Incidence of HWD in the US is increasing both within endemic areas and into erstwhile HW-free, Westerly and Northerly regions, including Canada (Simon et al., 2012). A similar epidemiological pattern of increased dirofilariae incidence in the Mediterranean and spread into Northern latitudes of Central and Western Europe has also been documented (Genchi and Kramer, 2017; Morchon et al., 2012). More anecdotal epideiological surveys exist for canid and feline filarid prevalences in Latin America, Africa and South East Asia but which highlight the potential for remarkably high incidence of mf positive animals (Simon et al., 2012). Recent surveys in Sri-Lanka and Thailand have noted, for example, prevalence of Dirofilaria and Brugia co-infections in both cats and dogs exceeding 60% (Mallawarachchi et al., 2018a). High incidence of zoonotic Brugia infections are thought to be a reservoir for persistent and re-emergent brugian lymphatic filariasis in health districts that have undergone extensive mass drug administration elimination programmes (Khowawisetsut et al., 2017; Mallawarachchi et al., 2018b). This also may suggest an underappreciated and underdiagnosed zoonotic infection potential for Dirofilaria spp. in humans in low to middle income countries.

As there is no vaccine available for dirofilariasis, disease is controlled by preventative chemotherapy and curative treatment of diagnosed cases. Because human zoonotic infections remain undetected until the occurrence of pathology, when immature stages have arrested, effective chemotherapeutic control of dirofilariasis in companion animals is also important to limit incidence of zoonotic human disease.

Prophylactic treatment of pets with macrocyclic lactones (ML), namely: ivermectin, milbimycin oxime, moxidectin and selamectin, are effective at targeting L3-L4 larvae during subcutaneous tissue development and before immature adults reach the pulmonary artery to establish adult infection and cause HWD in the case of D. immitis, reviewed by (Prichard and Geary, 2019; Wolstenholme et al., 2015). With oral ‘chewable’ ML formulations, a single monthly low dose regimen of an ML prophylactic effectively targets L3 and early-stage L4 in the subcutaneous tissues with complete efficacy proven in both experimental studies and dirofilariasis field trials, for instance see: (Clark et al., 1992; Di Cesare et al., 2014; Kryda et al., 2019; Pollono et al., 1998). More intermittent oral or topical treatments, with longer intervals between doses, are not currently recommended due to risk of failure to completely remove later stage L4 larvae (McCall et al., 1996; Paul et al., 1986). This approach is therefore vulnerable to incomplete adherence by pet owners or disrupted drug absorptions, particularly in areas where pets are exposed to year-round transmission. An injectable high-dose microsphere formulation of moxidectin, which exploits the lipophilicity of the drug to produce a subcutaneous depot and slow release exposure, have been proven efficacious to protect against the establishment of heartworm up to 12-months post-inoculation (McTier et al., 2019). Whilst long-lasting injectable formulations guard against the disruption of the prophylactic window, their increased expense, requirement for veterinary administration and the inability to conveniently combine in a single delivery with gut deworming anthelmintics which still generally require monthly oral treatments, reduces their widespread adoption as replacements of more frequent oral ML prophylaxis.

After more than 35 years of use in veterinary medicine, ML drug resistance is prevalent in veterinary nematode parasites, discussed in (Prichard and Geary, 2019). Monthly, repetitive and incomplete low dose ML exposure is a likely selection pressure for the establishment of ML resistance in Dirofilaria. Phenotypic resistance to ML in D. immitis was first formally demonstrated in a HW infected dog re-housed from New Orleans to Canada, whereby multiple high doses of ivermectin and milbimycin oxime were ineffective at clearing circulating mf (Bourguinat et al., 2011a). Subsequently, mf derived from individual client-owned HW positive dogs with loss of ML efficacy at the level of circulating mf have been passaged experimentally in laboratory maintained A. aegypti mosquitoes and dogs to develop multiple D. immitis ‘isolates’ (Pulaski et al., 2014; Snyder et al., 2011). Several isolates have now been formally determined to be resistant to the prophylactic mode-of-action of ML whereby timed experimental infections and accurate prophylactic dosing with ML have failed to prevent the development of fecund adult HW infections. At the genetic level, a repertoire of multiple single nucleotide polymorphisms (SNP) have been identified in mf that occur with higher frequency in infections that do not respond to ML (Bourguinat et al., 2015).

Once a dog is diagnosed with HWD, the only currently recommended curative ‘adulticidal’ treatment is injection with melarsomine dihydrochloride, an arsenical compound. Melarsomine has low selective toxicity and induces adverse reactions around the injection site. Further, there is risk of severe treatment adverse reactions following drug-mediated death of parasites causing potentially fatal thromboembolisms (Kramer et al., 2011; Simon et al., 2012). For these reasons, melarsomine is delivered as one low dose followed 30 days later by two similar low doses administered 24 h apart (AHS, 2019a). Further, prior to treatment initiation, dogs undergo a rigorous pre-assessment and high worm burdens (assessed by radiography or echocardiogram) may preclude treatment. Dogs need to be restricted from exercise over the course of treatment. Corticosteroid treatments may be administered to reduce inflammation during death of adult worms. Because of increased risk of anaphylactic complications and low selective toxicity, melarsomine is not recommended for the treatment of feline dirofilariasis (AHS, 2019b).

Recently, targeting of Wolbachia—an intracellular bacterium of some filarial species required for larval development and embryogenesis—with antibiotics has emerged as a strategy for treating filarial worm infection.

The prophylactic potential in certain filarial infections of targeting Wolbachia was first demonstrated in 1993 where gerbils administered tetracycline adlibitum in drinking water were completely protected from developing patent adult Brugia pahangi infections following subcutaneous experimental inoculations of L3 (Bosshardt et al., 1993). The prophylactic window was defined as constant exposure to tetracycline over the first 26 days of infection. In a follow-up investigation, 28-day dosing commencing at 14 days-post infection (when B. pahangi had established L4 stage larval infections) confirmed that a minimum 28-day ad libitum exposure to tetracycline in drinking water mediated prevention of mature female adult infections.

To date effective depletion of the endosymbiont Wolbachia in L3 or L4 developing larvae using tetracycline antibiotics can prevent adult filarial infections. However, long courses of 26-30 days are necessary to mediate complete prevention.

PCT publication WO 2018/134685 (filed 17 Jan. 2018) describes compounds with anti-Wolbachia activity shown to be effective in reducing Brugia malayi microfilaria production in an in vivo mouse model.

Due to emergent spread of Dirofilaria infection, the growing concerns of ML prophylactic failure in USA and the current inadequacies of curative treatments, there is a need for new therapeutic strategies for the treatment and prevention of Dirofilaria infection in mammals. Suitably, new therapeutic strategies would be provide prophylaxis and/or curative treatment without prolonged administration, e.g. single dose or short-course monthly delivery.

### SUMMARY OF THE INVENTION

In one aspect, the present invention provides a compound of formula I as defined herein, or a pharmaceutically acceptable salt or solvate thereof, for use in the treatment or prevention of a Dirofilaria infection in a mammal.

In another aspect, the present invention provides a method of treating or preventing a Dirofilaria infection in a mammal comprising administering to said mammal an effective amount of a compound of formula I as defined herein, or a pharmaceutically acceptable salt or solvate thereof.

In another aspect, the present invention provides the use of a compound of formula I as defined herein, or a pharmaceutically acceptable salt or solvate thereof, for treating or preventing a Dirofilaria infection in a mammal.

In another aspect, the present invention provides the use of a compound of formula I as defined herein, or a pharmaceutically acceptable salt or solvate thereof, in the manufacture of medicament for the treatment or prevention of a Dirofilaria infection in a mammal.

In another aspect, the present invention provides a compound of formula I as defined herein, or a pharmaceutically acceptable salt or solvate thereof, for use in the treatment or prevention of a disease or condition caused by a Dirofilaria infection in a mammal.

In another aspect, the present invention provides a method of treating or preventing a disease or condition caused by a Dirofilaria infection in a mammal comprising administering to said mammal an effective amount of a compound of formula I as defined herein, or a pharmaceutically acceptable salt or solvate thereof.

In another aspect, the present invention provides the use of a compound of formula I as defined herein, or a pharmaceutically acceptable salt or solvate thereof, for treating or preventing a disease or condition caused by a Dirofilaria infection in a mammal.

In another aspect, the present invention provides the use of a compound of formula I as defined herein, or a pharmaceutically acceptable salt or solvate thereof, in the manufacture of medicament for the treatment or prevention of a disease or condition caused by a Dirofilaria infection in a mammal.

Preferred, suitable, and optional features of any one particular aspect of the present invention are also preferred, suitable, and optional features of any other aspect.

### DETAILED DESCRIPTION OF THE INVENTION

### Definitions

The compounds described herein may be named according to either the IUPAC (International Union for Pure and Applied Chemistry) or CAS (Chemical Abstracts Service) nomenclature systems. It should be understood that unless expressly stated to the contrary, the terms “compounds of Formula I” and the more general term “compounds” refer to and include any and all compounds described by and/or with reference to Formula I. It should also be understood that these terms encompasses all stereoisomers, i.e. cis and trans isomers, as well as optical isomers, i.e. R and S enantiomers, of such compounds and all salts thereof, in substantially pure form and/or any mixtures of the foregoing in any ratio. This understanding extends to pharmaceutical compositions and methods of treatment that employ or comprise one or more compounds of the Formula I, either by themselves or in combination with additional agents.

The various hydrocarbon-containing moieties provided herein may be described using a prefix designating the minimum and maximum number of carbon atoms in the moiety, e.g. “(C_{a-b})” or “Cₐ-C_{b}” or “(a-b)C”. For example, (C_{a-b})alkyl indicates an alkyl moiety having the integer “a” to the integer “b” number of carbon atoms, inclusive. Certain moieties may also be described according to the minimum and maximum number of members with or without specific reference to a particular atom or overall structure. For example, the terms “a to b membered ring” or “having between a to b members” refer to a moiety having the integer “a” to the integer “b” number of atoms, inclusive.

“About” when used herein in conjunction with a measurable value such as, for example, an amount or a period of time and the like, is meant to encompass reasonable variations of the value, for instance, to allow for experimental error in the measurement of said value.

As used herein by themselves or in conjunction with another term or terms, “alkyl” and “alkyl group” refer to a branched or unbranched saturated hydrocarbon chain. Unless specified otherwise, alkyl groups typically contain 1-10 carbon atoms, such as 1-6 carbon atoms or 1-4 carbon atoms or 1-3 carbon atoms, and can be substituted or unsubstituted. Representative examples include, but are not limited to, methyl, ethyl, n-propyl, i-propyl, n-butyl, i-butyl, s-butyl, t-butyl, n-pentyl, n-hexyl, n-heptyl, n-octyl, n-nonyl, n-decyl, isopropyl, tert-butyl, isobutyl, etc.

As used herein by themselves or in conjunction with another term or terms, “alkylene” and “alkylene group” refer to a branched or unbranched saturated hydrocarbon chain. Unless specified otherwise, alkylene groups typically contain 1-10 carbon atoms, such as 1-6 carbon atoms or 1-3 carbon atoms, and can be substituted or unsubstituted. Representative examples include, but are not limited to, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₃—, and —CH₂CH₂CH₂—), etc.

As used herein by themselves or in conjunction with another term or terms, “alkenyl” and “alkenyl group” refer to a branched or unbranched hydrocarbon chain containing at least one double bond. Unless specified otherwise, alkenyl groups typically contain 2-10 carbon atoms, such as 2-6 carbon atoms or 2-4 carbon atoms, and can be substituted or unsubstituted. Representative examples include, but are not limited to, ethenyl, 3-buten-1-yl, 2-ethenylbutyl, and 3-hexen-1-yl.

As used herein by themselves or in conjunction with another term or terms, “alkynyl” and “alkynyl group” refer to a branched or unbranched hydrocarbon chain containing at least one triple bond. Unless specified otherwise, alkynyl groups typically contain 2-10 carbon atoms, such as 2-6 carbon atoms or 2-4 carbon atoms, and can be substituted or unsubstituted. Representative examples include, but are not limited to, ethynyl, 3-butyn-1-yl, propynyl, 2-butyn-1-yl, and 3-pentyn-1-yl.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

As used herein by themselves or in conjunction with another term or terms, “aryl” and “aryl group” refer to phenyl and 7-15 membered bicyclic or tricyclic hydrocarbon ring systems, including bridged, spiro, and/or fused ring systems, in which at least one of the rings is aromatic. Aryl groups can be substituted or unsubstituted. Unless specified otherwise, an aryl group may contain 6 ring atoms (i.e., phenyl) or a ring system containing 9 to 15 atoms, such as 9 to 11 ring atoms, or 9 or 10 ring atoms. Representative examples include, but are not limited to, naphthyl, indanyl, 1,2,3,4-tetrahydronaphthalenyl, 6,7,8,9-tetrahydro-5H-benzocycloheptenyl, and 6,7,8,9-tetrahydro-5H-benzocycloheptenyl. Suitably an aryl group is phenyl and naphthyl, suitably phenyl.

As used herein by themselves or in conjunction with another term or terms, “arylene” and “arylene group” refer to a phenylene (—C₆H₄—) or to 7 to 15 membered bicyclic or tricyclic hydrocarbon ring systems, including bridged, spiro, and/or fused ring systems, in which at least one of the rings is aromatic. Arylene groups can be substituted or unsubstituted. In some embodiments, an arylene group may contain 6 (i.e., phenylene) ring atoms or be a ring system containing 9 to 15 atoms; such as 9 to 11 ring atoms; or 9 or 10 ring atoms. Arylene groups can be substituted or unsubstituted.

As used herein by themselves or in conjunction with another term or terms, “alkylaryl” and “arylalkyl group” refer to an alkyl group in which a hydrogen atom is replaced by an aryl group, wherein alkyl group and aryl group are as previously defined, such as, for example, benzyl (C₆H₅CH₂—). Arylalkyl groups can be substituted or unsubstituted.

As used herein by themselves or in conjunction with another term or terms, “carbocyclic group” and “carbocycle” refer to monocyclic and polycyclic ring systems that contain only carbon atoms in the ring(s), i.e., hydrocarbon ring systems, without regard or reference to aromaticity or degree of unsaturation. Thus, carbocyclic group should be understood as referring to and including ring systems that are fully saturated (such as, for example, a cyclohexyl group), ring systems that are aromatic (such as, for example, a phenyl group), as well as ring systems having fully saturated, aromatic and/or unsaturated portions (such as, for example, cyclohexenyl, 2,3-dihydro-indenyl, and 1,2,3,4-tetrahydro-naphthalenyl). The terms carbocyclic and carbocycle further include bridged, fused, and spirocyclic ring systems.

As used herein by themselves or in conjunction with another term or terms, “cycloalkyl” and “cycloalkyl group” refer to a non-aromatic carbocyclic ring system, that may be monocyclic, bicyclic, or tricyclic, saturated or unsaturated, and may be bridged, spiro, and/or fused. A cycloalkyl group may be substituted or unsubstituted. Unless specified otherwise, a cycloalkyl group typically contains from 3 to 12 ring atoms. In some instances a cycloalkyl group may contain 4 to 10 ring atoms (e.g., 4 ring atoms, 5 ring atoms, 6 ring atoms, 7 ring atoms, etc.). Representative examples include, but are not limited to, cyclopropyl, cyclopropenyl, cyclobutyl, cyclobutenyl, cyclopentyl, cyclopentenyl, cyclohexyl, cyclohexenyl, norbornyl, norbornenyl, bicyclo[2.2.1]hexane, bicyclo[2.2.1]heptane, bicyclo[2.2.1]heptene, bicyclo[3.1.1]heptane, bicyclo[3.2.1]octane, bicyclo[2.2.2]octane, bicyclo[3.2.2]nonane, bicyclo[3.3.1]nonane, and bicyclo[3.3.2]decane. Suitably, cycloalkyl groups are selected from cyclopropyl, cyclobutyl, cyclopentyl and cyclohexyl groups.

As used herein by themselves or in conjunction with another term or terms, “cycloalkylalkyl” and “cycloalkylalkyl group” refer to an alkyl group in which a hydrogen atom is replaced by a cycloalkyl group, wherein alkyl group and cycloalkyl group are as previously defined, such as, for example, cyclohexylmethyl (C₆H₁₁CH₂—). Cycloalkylalkyl groups can be substituted or unsubstituted.

As used herein by themselves or in conjunction with another term or terms, “haloalkyl” and “haloalkyl group” refer to alkyl groups in which one or more hydrogen atoms are replaced by halogen atoms. Haloalkyl includes both saturated alkyl groups as well as unsaturated alkenyl and alkynyl groups. Representative examples include, but are not limited to, —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —Cl═CH₂, —C≡C—CF₃, —CHFCH₂CH₃ and —CHFCH₂CF₃. Haloalkyl groups can be substituted or unsubstituted. Suitably, a haloalkyl group is selected from CHF₂ and CF₃, suitably CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Haloalkoxy includes both saturated alkoxy groups as well as unsaturated alkenyl and alkynyl groups. Representative examples include, but are not limited to, —OCF₃, —OCHF₂, —OCH₂F, —OCF₂CF₃, —OCHFCF₃, —OCH₂CF₃, —OCF₂CH₃, —OCHFCH₃, —OCF₂CF₂CF₃, —OCF₂CH₂CH₃, —OCF═CF₂, —OCCl═CH₂, —OCBr═CH₂, —OCHFCH₂CH₃ and —OCHFCH₂CF₃. Haloalkoxy groups can be substituted or unsubstituted. Suitably, a haloalkyoxy group is selected from —OCHF₂ and —OCF₃, suitably —OCF₃.

As used herein by themselves or in conjunction with another term or terms, “halo” and “halogen” include fluorine, chlorine, bromine and iodine atoms and substituents.

As used herein by themselves or in conjunction with another term or terms, “heteroaryl” and “heteroaryl group” refer to (a) 5 and 6 membered monocyclic aromatic rings, which contain, in addition to carbon atom(s), at least one heteroatom, such as nitrogen, oxygen or sulfur, and (b) 7 to 15 membered bicyclic and tricyclic rings, which contain, in addition to carbon atom(s), at least one heteroatom, such as nitrogen, oxygen or sulfur, and in which at least one of the rings is aromatic. In some instances, a heteroaryl group can contain two or more heteroatoms, which may be the same or different. Heteroaryl groups can be substituted or unsubstituted, and may be bridged, spiro, and/or fused. In some instances, a heteroaryl group may contain 5, 6, or 8 to 15 ring atoms. In other instances, a heteroaryl group may contain 5 to 10 ring atoms, such as 5, 6, 9, or 10 ring atoms. Representative examples include, but are not limited to, 2,3-dihydrobenzofuranyl, 1,2-dihydroquinolinyl, 3,4-dihydroisoquinolinyl, 1,2,3,4-tetrahydroisoquinolinyl, 1,2,3,4-tetrahydroquinolinyl, benzoxazinyl, benzthiazinyl, chromanyl, furanyl, 2-furanyl, 3-furanyl, imidazolyl, isoxazolyl, isothiazolyl, oxadiazolyl, oxazolyl, pyridinyl, 2-, 3-, or 4-pyridinyl, pyrimidinyl, 2-, 4-, or 5-pyrimidinyl, pyrazolyl, pyrrolyl, 2- or 3-pyrrolyl, pyrazinyl, pyridazinyl, 3- or 4-pyridazinyl, 2-pyrazinyl, thienyl, 2-thienyl, 3-thienyl, tetrazolyl, thiazolyl, thiadiazolyl, triazinyl, triazolyl, pyridin-2-yl, pyridin-4-yl, pyrimidin-2-yl, pyridazin-4-yl, pyrazin-2-yl, naphthyridinyl, pteridinyl, phthalazinyl, purinyl, alloxazinyl, benzimidazolyl, benzofuranyl, benzofurazanyl, 2H-1-benzopyranyl, benzothiadiazine, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl, isoquinolinyl, 10-aza-tricyclo[6.3.1.0^{2,7}]dodeca-2(7),3,5-trienyl, 12-oxa-10-aza-tricyclo[6.3.1.0^{2,7}]dodeca-2(7),3,5-trienyl, 12-aza-tricyclo[7.2.1.0^{2,7}]dodeca-2(7),3,5-trienyl, 10-aza-tricyclo[6.3.2.0^{2,7}]trideca-2(7),3,5-trienyl, 2,3,4,5-tetrahydro-1H-benzo[d]azepinyl, 1,3,4,5-tetrahydro-benzo[d]azepin-2-onyl, 1,3,4,5-tetrahydro-benzo[b]azepin-2-onyl, 2,3,4,5-tetrahydro-benzo[c]azepin-1-onyl, 1,2,3,4-tetrahydro-benzo[e][1,4]diazepin-5-onyl, 2,3,4,5-tetrahydro-1H-benzo[e][1,4]diazepinyl, 5,6,8,9-tetrahydro-7-oxa-benzocycloheptenyl, 2,3,4,5-tetrahydro-1H-benzo[b]azepinyl, 1,2,4,5-tetrahydro-benzo[e][1,3]diazepin-3-onyl, 3,4-dihydro-2H-benzo[b][1,4]dioxepinyl, 3,4-dihydro-2H-benzo[f][1,4]oxazepin-5-onyl, 6,7,8,9-tetrahydro-5-thia-8-aza-benzocycloheptenyl, 5,5-dioxo-6,7,8,9-tetrahydro-5-thia-8-aza-benzocycloheptenyl, and 2,3,4,5-tetrahydro-benzo[f][1,4]oxazepinyl. Suitably, a heteroaryl is a 5- or 6-membered heteroaryl ring comprising one, two or three heteroatoms selected from N, O or S.

As used herein by themselves or in conjunction with another term or terms, “heteroarylalkyl” and “heteroarylalkyl group” refer to an alkyl group in which a hydrogen atom is replaced by a heteroaryl group, wherein alkyl group and heteroaryl group are as previously defined. Heteroarylalkyl groups can be substituted or unsubstituted. Where carbon numbers are provided, e.g. (Cₙ₋ₘ) heteroarylalkyl, the range refers to the whole group. Suitably, the constituent alkyl group has 1-6 carbons, suitable 1-3 carbons.

As used herein by themselves or in conjunction with another term or terms, “heterocyclic group” and “heterocycle” refer to monocyclic and polycyclic ring systems that contain carbon atoms and at least one heteroatom selected from nitrogen, oxygen, sulfur or phosphorus in the ring(s), without regard or reference to aromaticity or degree of unsaturation. Thus, a heterocyclic group should be understood as referring to and including ring systems that are fully saturated (such as, for example, a piperidinyl group), ring systems that are aromatic (such as, for example, a pyrindinyl group), as well as ring systems having fully saturated, aromatic and/or unsaturated portions (such as, for example, 1,2,3,6-tetrahydropyridinyl and 6,8-dihydro-5H-[1,2,4]triazolo[4,3-a]pyrizinyl). The terms heterocyclic and heterocycle further include bridged, fused, and spirocyclic ring systems.

As used herein by themselves or in conjunction with another term or terms, “heterocycloalkyl” and “heterocycloalkyl group” refer to 3 to 15 membered monocyclic, bicyclic, and tricyclic non-aromatic ring systems, which contain, in addition to carbon atom(s), at least one heteroatom, such as nitrogen, oxygen, sulfur or phosphorus. Heterocycloalkyl groups may be fully saturated or contain unsaturated portions and may be bridged, spiro, and/or fused ring systems. In some instances a heterocycloalkyl group may contain at least two or heteroatoms, which may be the same or different. Heterocycloalkyl groups can be substituted or unsubstituted. In some instances a heterocycloalkyl group may contain from 3 to 10 ring atoms or from 3 to 7 ring atoms or from 5 to 7 ring atoms, such as 5 ring atoms, 6 ring atoms, or 7 ring atoms. Representative examples include, but are not limited to, tetrahydrofuranyl, pyrrolidinyl, pyrrolinyl, imidazolidinyl, imidazolinyl, pyrazolidinyl, pyrazolinyl, piperidyl, piperazinyl, indolinyl, isoindolinyl, morpholinyl, thiomorpholinyl, homomorpholinyl, homopiperidyl, homopiperazinyl, thiomorpholinyl-5-oxide, thiomorpholinyl-S,S-dioxide, pyrrolidinyl, tetrahydropyranyl, piperidinyl, tetrahydrothienyl, homopiperidinyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, quinuclidinyl, 2-oxa-5-azabicyclo[2.2.1]heptanyl, 8-oxa-3-aza-bicyclo[3.2.1]octanyl, 3,8-diaza-bicyclo[3.2.1]octanyl, 2,5-diaza-bicyclo[2.2.1]heptanyl, 3,8-diaza-bicyclo[3.2.1]octanyl, 3,9-diaza-bicyclo[4.2.1]nonanyl, 2,6-diaza-bicyclo[3.2.2]nonanyl, [1,4]oxaphosphinanyl-4-oxide, [1,4]azaphosphinanyl-4-oxide, [1,2]oxaphospholanyl-2-oxide, phosphinanyl-1-oxide, [1,3]azaphospholidinynl-3-oxide, [1,3]oxaphospholanyl-3-oxide, 7-oxabicyclo[2.2.1]heptanyl, 6,8-dihydro-5H-[1,2,4]triazolo[4,3-a]pyrazin-7-yl, 6,8-dihydro-5H-imidazo[1,5-a]pyrazin-7-yl, 6,8-dihydro-5H-imidazo[1,2-a]pyrazin-7-yl, 5,6,8,9-tetrahydro-[1,2,4]triazolo[4,3-d][1,4]diazepin-7-yl and 6,8-dihydro-5H-[1,2,4]triazolo[4,3-a]pyrazin-7-yl. Suitably, a heterocyclylalkyl group as defined herein is a monocyclic, bicyclic or spiro heterocyclyl group comprising one, two or three heteroatoms selected from N, O or S.

As used herein by themselves or in conjunction with another term or terms, “heterocycloalkylene” and “heterocycloalkylene group” refer to 3 to 15 membered monocyclic, bicyclic, or tricyclic non-aromatic ring systems, which contain, in addition to carbon atom(s), at least one heteroatom, such as nitrogen, oxygen, sulfur or phosphorus. Heterocycloalkylene groups may be fully saturated or contain unsaturated portions and may be bridged, spiro, and/or fused. Heterocycloalkylene groups can be substituted or unsubstituted. In some instances, a heterocycloalkylene group may contain from 3 to 10 ring atoms; such as from 3 to 7 ring atoms. In other instances a heterocycloalkylene group may contain from 5 to 7 ring atoms, such as 5 ring atoms, 6 ring atoms, or 7 ring atoms.

As used herein by themselves or in conjunction with another term or terms, “heterocycloalkylalkyl” and “heterocycloalkylalkyl group” refer to an alkyl group in which a hydrogen atom is replaced by a heterocycloalkyl group, wherein alkyl group and heterocycloalkyl group are as previously defined, such as, for example, pyrrolidinylmethyl (C₄H₈NCH₂—). Heteroycloalkylalkyl groups can be substituted or unsubstituted. Where carbon numbers are provided, e.g. (Cₙ₋ₘ) heterocycloalkylalkyl, the range refers to the whole group. Suitably, the constituent alkyl group has 1-6 carbons, suitable 1-3 carbons.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

As used herein by itself or in conjunction with another term or terms, “pharmaceutical composition” refers to a composition that can be used to treat a disease, condition, or disorder in a subject, including a human.

As used herein by itself or in conjunction with another term or terms, “pseudohalogen” refers to —OCN, —SCN, —CF₃, and —CN.

As used herein by themselves or in conjunction with another term or terms, “stable” and “chemically stable” refer to a compound that is sufficiently robust to be isolated from a reaction mixture with a useful degree of purity. The present application is directed solely to the preparation of stable compounds. When lists of alternative substituents include members which, owing to valency requirements, chemical stability, or other reasons, cannot be used to substitute a particular group, the list is intended to be read in context to include those members of the list that are suitable for substituting the particular group. For example, when considering the degree of optional substitution of a particular moiety, it should be understood that the number of substituents does not exceed the valency appropriate for that moiety. For example, if R¹ is a methyl group (—CH₃), it can be optionally substituted by 1 to 3 R⁵.

As used herein by itself or in conjunction with another term or terms, “substituted” indicates that a hydrogen atom on a molecule has been replaced with a different atom or group of atoms and the atom or group of atoms replacing the hydrogen atom is a “substituent.” It should be understood that the terms “substituent”, “substituents”, “moiety”, “moieties”, “group”, or “groups” refer to substituent(s).

As used herein by themselves or in conjunction with another term or terms, “treatment” or “treating” refer to an action (e.g. compound or composition for administration) which (a) inhibits or causes an improvement in a particular disease, condition or disorder; (b) attenuates, ameliorates or eliminates one or more symptoms of a particular disease, condition or disorder; (c) delays the onset of one or more symptoms of a particular disease, condition or disorder described herein or (d) diminishes the likelihood or seriousness of a condition, symptom, or disease state. It should be understood that the terms “therapeutic” and “therapeutically effective” encompass any one of the aforementioned effects (a)-(d), either alone or in combination with any of the others (a)-(d). It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein whether by themselves or in conjunction with another term or terms, “prevention” or “preventing”, refers to prophylactic treatment. Prevention as used herein is not absolute but rather refers to uses and results where the administration of a compound or composition diminishes the likelihood or seriousness of a condition, symptom, or disease state, and/or delays the onset of a condition, symptom, or disease state for a period of time.

A “therapeutically effective amount” means the amount of a compound that, when administered to a subject or patient for treating a disease, is sufficient to effect such treatment for the disease. The “therapeutically effective amount” will vary depending on the compound, the disease and its severity and the age, weight, etc., of the subject or patient to be treated. Similarly, a “prophylactically effective amount” means the amount of a compound that, when administered to a subject or patient (e.g. mammal) for preventing a disease, is sufficient to effect such prevention of the disease.

The term “drug resistance” or “drug resistant” and the like, as used herein, unless otherwise indicated, refers to the ability of a parasite to display a delayed, lessened and/or null response to treatment or prophylaxis with a therapeutic agent (e.g. a macrocyclic lactone or tetracycline antibiotic) at the therapeutically recommended dosages, which would normally treat or protect against said parasites of the same species and stage. The term is used to include such separately identifiable forms of resistance as “full resistance”, “immunity”, “partial resistance”, “hypersensitivity” and “tolerance”.

As used herein, Dirofilaria infection (or dirofilarial infection) refers to infection with a parasitic worm from the genus Dirofilaria.

### Medical Uses and Methods of Treatment

In one aspect, the present invention provides a compound of formula I as defined herein, or a pharmaceutically acceptable salt or solvate thereof, for use in the treatment or prevention of a Dirofilaria infection in a mammal.

In another aspect, the present invention provides a method of treating or preventing a Dirofilaria infection in a mammal comprising administering to said mammal an effective amount of a compound of formula I as defined herein, or a pharmaceutically acceptable salt or solvate thereof.

In another aspect, the present invention provides the use of a compound of formula I as defined herein, or a pharmaceutically acceptable salt or solvate thereof, for treating or preventing a Dirofilaria infection in a mammal.

In another aspect, the present invention provides the use of a compound of formula I as defined herein, or a pharmaceutically acceptable salt or solvate thereof, in the manufacture of medicament for the treatment or prevention of a Dirofilaria infection in a mammal.

In each of the aforementioned aspects of the invention, the compound of formula I, is defined as follows:

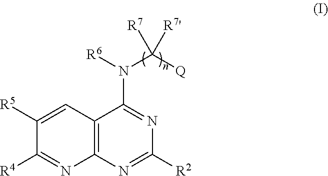

wherein
Q is a group selected from an C₃₋₁₁cycloalkyl optionally substituted by one or more R^{b}, 3-15 membered heterocycloalkyl optionally substituted by one or more R^{b}, C₆₋₁₁ aryl group optionally substituted with by one or more R^{b}, 5-15 membered heteroaryl optionally substituted by one or more R^{b};
R⁶ is selected from hydrogen and C₁₋₆ alkyl;
R⁷ and R^{7′} are independently selected from hydrogen, C₃₋₆ cycloalkyl, C₁₋₆ alkyl, where said C₃₋₆ cycloalkyl and C₁₋₆ alkyl are optionally substituted by one or more R^{a}; or
R⁷ and R^{7′}, together with the carbon to which they are attached form a 3-7 membered cycloalkyl ring, optionally substituted with one or more R^{a}, or R⁷ and R^{7′}, together with the carbon to which they are attached form a carbonyl group; or
R⁶ and R⁷, together with the atoms to which they are attached form a 3-7 membered heterocyclic ring, optionally substituted with one or more R^{a};
n is a number selected from 1, 2 and 3;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1} OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1}, C₁₋₁₀ haloalkyl, C₁₋₁₀alkyl optionally substituted by one or more R^{e}, C₂₋₆alkenyl optionally substituted by one or more R^{e}, C₂₋₆alkynyl optionally substituted by one or more R^{e}, C₆₋₁₁aryl optionally substituted by one or more R^{e}, (C₇₋₁₆)alkylaryl optionally substituted by one or more R^{e}, C₃₋₁₁cycloalkyl optionally substituted by one or more R^{e}, (C₄₋₁₇)cycloalkylalkyl optionally substituted by one or more R^{e}, 3-15 membered heterocycloalkyl optionally substituted by one or more R^{e}, 4-21 membered alkylheterocycloalkyl optionally substituted by one or more R^{e}, 5-15 membered heteroaryl optionally substituted by one or more R^{e}, and 6-21 membered alkylheteroaryl optionally substituted by one or more R^{e};
where each R^{c1} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d1} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c1} and R^{d1}, when attached to the same atom, together with the atom to which they are attached form a 3-7 membered ring, optionally containing one or more for heteroatoms selected from O, NH and S, and wherein said ring is optionally substituted with one or more R^{a};
where each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl and 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆alkyl;
each R^{b} and R^{e} is independently selected from hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c2} and R^{d2}, when attached to the same atom, together with the atom to which they are attached form a 3-7 membered ring, optionally containing one or more for heteroatoms selected from O, NH and S, and wherein said ring is optionally substituted with one or more R^{a};
R³, R⁴ and R⁵ are independently selected from hydrogen, hydroxyl, halogen, CN, C₁₋₆ haloalkyl, C₁₋₆ haloalkoxy, C₁₋₆ alkyl, phenyl and cyclopropyl, wherein said C₁₋₆ alkyl, phenyl and cyclopropyl are optionally substituted by one or more R^{a}.

In each of the aforementioned aspects, in one embodiment the Dirofilaria infection is an infection with a Dirofilaria spp. parasite. Typically, the Dirofilaria parasite is selected from one or more of the species D. immitis D. repens and D. tenuis. Accordingly, in one embodiment, the Dirofilaria infection is an infection with one or more of D. immitis D. repens and D. tenuis.

In one embodiment, the Dirofilaria parasite is selected from D. immitis and/or D. repens. Suitably, the Dirofilaria infection is an infection with one or more of D. immitis and D. repens. Suitably, the Dirofilaria infection is D. immitis and/or D. repens infection.

In one embodiment, the Dirofilaria infection is infection with a single Dirofilaria parasite, suitably selected from D. immitis, D. repens and D. tenuis. In another embodiment, the single Dirofilarial parasite is suitably selected from D. immitis and D. repens. In another embodiment, the single Dirofilaria parasite is D. immitis. In another embodiment, the single Dirofilarial parasite is D. repens.

In one embodiment, the dirofilarial infection comprises D. immitis infection. In another embodiment, the Dirofilaria infection consists of D. immitis infection.

In one embodiment, the D. immitis infection is a resistant D. immitis infection, i.e. infection with a drug resistant D. immitis parasite. For instance, in one embodiment, the resistant D. immitis parasite is resistant to treatment or prophylaxis with a macrocyclic lactone or tetracycline antibiotic when either is used alone. Suitably, the D. immitis parasite is resistant to treatment or prophylaxis with a macrocyclic lactone when used alone.

In one embodiment, the Dirofilaria infection is infection with a Dirofilaria microfilariae, Dirofilaria third stage (L3) larvae, Dirofilaria fourth stage (L4) larvae, Dirofilaria fifth stage (L5) larvae, an adult dirofilarial worm or a combination thereof.

In one embodiment, the Dirofilaria infection is infection with a Dirofilaria microfilariae, Dirofilaria third stage (L3) larvae or Dirofilaria fourth stage (L4) larvae, or a combination thereof.

In another embodiment, the Dirofilaria infection is infection with a Dirofilaria third stage (L3) larvae or Dirofilaria fourth stage (L4) larvae, or a combination thereof.

In one embodiment, the Dirofilaria infection is infection with a D. immitis microfilariae, D. immitis third stage (L3) larvae, D. immitis fourth stage (L4) larvae, D. immitis fifth stage (L5) larvae, an adult D. immitis worm or a combination thereof.

In one embodiment, the Dirofilaria infection is infection with a D. immitis microfilariae, D. immitis third stage (L3) larvae, a D. immitis fourth stage (L4) larvae, or a combination thereof.

In another embodiment, the Dirofilaria infection is infection with a D. immitis third stage (L3) larvae or a D. immitis fourth stage (L4) larvae, or a combination thereof.

In one embodiment, the Dirofilaria infection is infection with a D. repens microfilariae, D. repens third stage (L3) larvae, D. repens fourth stage (L4) larvae, D. repens fifth stage (L5) larvae, an adult D. repens worm or a combination thereof.

In one embodiment, the Dirofilaria infection is infection with a D. repens microfilariae, D. repens third stage (L3) larvae or a D. repens fourth stage (L4) larvae, or a combination thereof.

In another embodiment, the Dirofilaria infection is infection with a D. repens third stage (L3) larvae or a D. repens fourth stage (L4) larvae, or a combination thereof.

In one embodiment, the Dirofilaria infection is infection with a D. tenuis microfilariae, D. tenuis third stage (L3) larvae, D. tenuis fourth stage (L4) larvae, D. tenuis fifth stage (L5) larvae an adult D. tenuis worm or a combination thereof.

In one embodiment, the Dirofilaria infection is infection with a D. tenuis microfilariae, D. tenuis third stage (L3) larvae or a D. tenuis fourth stage (L4) larvae, or a combination thereof.

In another embodiment, the Dirofilaria infection is infection with a D. tenuis third stage (L3) larvae or a D. tenuis fourth stage (L4) larvae, or a combination thereof.

In one embodiment of any of the aforementioned aspects, the mammal is selected from a human, a dog, a cat, a racoon, a coyote, a jackal, a wolf, a fox, and a ferret. In another embodiment, the mammal is selected from a human, a dog, a cat, a racoon and a ferret. In another embodiment, the mammal is selected from a human, a dog and a cat. In another embodiment, the mammal is selected from a dog and a cat. In another embodiment, the mammal is a dog.

In one embodiment, the Dirofilaria infection is D. immitis infection and the mammal is selected from a human, a dog, a cat, a racoon, a coyote, a jackal, a wolf, a fox, and a ferret. In another embodiment, the Dirofilaria infection is D. immitis infection and the mammal is selected from a human, a dog, a cat, a coyote, a jackal, a wolf, a fox, and a ferret. In another embodiment, the Dirofilaria infection is D. immitis infection and the mammal is selected from a human, a dog, a cat, a racoon and a ferret. In another embodiment, the Dirofilaria infection is D. immitis infection and the mammal is selected from a human, a dog, a cat, and a ferret. In another embodiment, the Dirofilaria infection is D. immitis infection and the mammal is selected from a human, a dog and a cat. In another embodiment, the Dirofilaria infection is D. immitis infection and the mammal is selected from a dog and a cat. In another embodiment, the Dirofilaria infection is D. immitis infection and the mammal is a dog.

In one embodiment, the Dirofilaria infection is D. repens infection and the mammal is selected from a human, a dog, a cat, a racoon, a coyote, a jackal, a wolf, a fox, and a ferret. In another embodiment, the Dirofilaria infection is D. repens infection and the mammal is selected from a human, a dog, a cat, a coyote, a jackal, a wolf, a fox, and a ferret. In another embodiment, the Dirofilaria infection is D. repens infection and the mammal is selected from a human, a dog, a cat, a racoon and a ferret. In another embodiment, the Dirofilaria infection is D. repens infection and the mammal is selected from a human, a dog, a cat, and a ferret. In another embodiment, the Dirofilaria infection is D. repens infection and the mammal is selected from a human, a dog and a cat. In another embodiment, the Dirofilaria infection is D. repens infection and the mammal is selected from a dog and a cat. In another embodiment, the Dirofilaria infection is D. repens infection and the mammal is a dog.

In another embodiment, the Dirofilaria infection is D. tenuis infection and the mammal is selected from a human and a racoon. In one embodiment, the Dirofilaria infection is D. tenuis infection and the mammal is a racoon. In one embodiment, the Dirofilaria infection is D. tenuis infection and the mammal is a human.

In one embodiment of each of the aforementioned aspects, the compound of formula I, or a pharmaceutically salt or solvate thereof, is administered/for administration for 7 days per month or less. Suitably, the compound of formula I, or a pharmaceutically salt or solvate thereof, is administered/for administration for 6, 5, 4, 3 or 2 days per month or less.

In one embodiment, the compound of formula I, or a pharmaceutically salt or solvate thereof, is administered/for administration for 7, 6, 5, 4, 3 or 2 days per month.

In one embodiment of each of the aforementioned aspects, the compound of formula I, or a pharmaceutically salt or solvate thereof, is administered/for administration for 7 consecutive days per month or less. Suitably, the compound of formula I, or a pharmaceutically salt or solvate thereof, is administered/for administration for 6, 5, 4, 3 or 2 consecutive days per month or less.

In one embodiment, the compound of formula I, or a pharmaceutically salt or solvate thereof, is administered/for administration for 7, 6, 5, 4, 3 or 2 consecutive days per month.

In one embodiment of each of the above aspects, the compound of formula I, or a pharmaceutically salt or solvate thereof, is administered/for administration once per month.

In one embodiment provided herein is a compound of formula I, or pharmaceutically acceptable salt or solvate thereof, for use in the treatment or prevention of D. immitis or D. repens infection in a mammal selected from a human, a dog or a cat, wherein the compound of formula I, or a pharmaceutically acceptable salt or solvate thereof is for administration for 7 days per month or less, suitably 7 consecutive days per month or less.

In one embodiment provided herein is a compound of formula I, or pharmaceutically acceptable salt or solvate thereof, for use in the treatment or prevention of D. immitis or D. repens infection in a mammal selected from a human, a dog or a cat, wherein the compound of formula I, or pharmaceutically acceptable salt or solvate thereof is for administration for 5 days per month or less, suitably 5 consecutive days per month or less.

In one embodiment provided herein is a compound of formula I, or pharmaceutically acceptable salt or solvate thereof, for use in the treatment or prevention of D. immitis or D. repens infection in a mammal selected from a human, a dog or a cat, wherein the compound of formula I, or pharmaceutically acceptable salt or solvate thereof is for administration for 1 or 2 days per month, suitably 1 or 2 consecutive days per month.

In one embodiment provided herein is a compound of formula I, or pharmaceutically acceptable salt or solvate thereof, for use in the treatment or prevention of D. immitis infection in a mammal selected from a human, a dog or a cat, wherein the compound of formula I, or pharmaceutically acceptable salt or solvate thereof is for administration for 7 days per month or less, suitably 7 consecutive days per month or less.

In one embodiment provided herein is a compound of formula I, or pharmaceutically acceptable salt or solvate thereof, for use in the treatment or prevention of D. immitis infection in a mammal selected from a human, a dog or a cat, wherein the compound of formula I, or pharmaceutically acceptable salt or solvate thereof is for administration for 5 days per month or less, suitably 5 consecutive days per month or less.

In one embodiment provided herein is a compound of formula I, or pharmaceutically acceptable salt or solvate thereof, for use in the treatment or prevention of D. immitis infection in a mammal selected from a human, a dog or a cat, wherein the compound of formula I, or pharmaceutically acceptable salt or solvate thereof is for administration for 1 or 2 days per month, suitably 1 or 2 consecutive days per month or less.

In one embodiment provided herein is a compound of formula I, or pharmaceutically acceptable salt or solvate thereof, for use in the treatment or prevention of D. immitis L3 or L4 infection in a mammal selected from a human, a dog or a cat, wherein the compound of formula I, or pharmaceutically acceptable salt or solvate thereof is for administration for 7 days per month or less, suitably 7 consecutive days per month or less.

In one embodiment provided herein is a compound of formula I, or pharmaceutically acceptable salt or solvate thereof, for use in the treatment or prevention of D. immitis L3 or L4 infection in a mammal selected from a human, a dog or a cat, wherein the compound of formula I, or pharmaceutically acceptable salt or solvate thereof is for administration for 5 days per month or less, suitably 5 consecutive days per month or less.

In one embodiment provided herein is a compound of formula I, or pharmaceutically acceptable salt or solvate thereof, for use in the treatment or prevention of D. immitis L3 or L4 infection in a mammal selected from a human, a dog or a cat, wherein the compound of formula I, or pharmaceutically acceptable salt or solvate thereof is for administration for 1 or 2 days per month, suitably 1 or 2 consecutive days per month or less.

In one embodiment provided herein is a compound of formula I, or pharmaceutically acceptable salt or solvate thereof, for use in the treatment or prevention of D. immitis L3 or L4 infection in a mammal selected from a human, a dog or a cat, wherein the compound of formula I, or pharmaceutically acceptable salt or solvate thereof is for administration for 1 or 2 days per month, suitably 1 or 2 consecutive days per month or less.

In one embodiment provided herein is a compound of formula I, or pharmaceutically acceptable salt or solvate thereof, for use in the treatment or prevention of D. immitis mf infection in a mammal selected from a human, a dog or a cat, wherein the compound of formula I, or pharmaceutically acceptable salt or solvate thereof is for administration for 7 days per month or less, suitably 7 consecutive days per month or less.

In one embodiment provided herein is a compound of formula I, or pharmaceutically acceptable salt or solvate thereof, for use in the treatment or prevention of D. immitis mf infection in a mammal selected from a human, a dog or a cat, wherein the compound of formula I, or pharmaceutically acceptable salt or solvate thereof is for administration for 5 days per month or less, suitably 5 consecutive days per month or less.

In one embodiment provided herein is a compound of formula I, or pharmaceutically acceptable salt or solvate thereof, for use in the treatment or prevention of D. immitis mf infection in a mammal selected from a human, a dog or a cat, wherein the compound of formula I, or pharmaceutically acceptable salt or solvate thereof is for administration for 1 or 2 days per month, suitably 1 or 2 consecutive days per month or less.

In one embodiment provided herein is a compound of formula I, or pharmaceutically acceptable salt or solvate thereof, for use in the treatment or prevention of D. immitis mf infection in a mammal selected from a human, a dog or a cat, wherein the compound of formula I, or pharmaceutically acceptable salt or solvate thereof is for administration for 1 or 2 days per month, suitably 1 or 2 consecutive days per month or less.

In one embodiment provided herein is a method for the treatment or prevention of D. immitis or D. repens infection in a mammal selected from a human, a dog or a cat, wherein said method comprises administering to said mammal a compound of formula I, or a pharmaceutically acceptable salt or solvate thereof for 7 days per month or less, suitably 7 consecutive days per month or less.

In one embodiment provided herein is a method for the treatment or prevention of D. immitis or D. repens infection in a mammal selected from a human, a dog or a cat, wherein said method comprises administering to said mammal a compound of formula I, or a pharmaceutically acceptable salt or solvate thereof for 5 days per month or less, suitably 5 consecutive days per month or less.

In one embodiment provided herein is a method for the treatment or prevention of D. immitis or D. repens infection in a mammal selected from a human, a dog or a cat, wherein said method comprises administering to said mammal a compound of formula I, or a pharmaceutically acceptable salt or solvate thereof for administration for 1 or 2 days per month, suitably 1 or 2 consecutive days per month.

In one embodiment provided herein is a method for the treatment or prevention of D. immitis infection in a mammal selected from a human, a dog or a cat, wherein said method comprises administering to said mammal a compound of formula I, or a pharmaceutically acceptable salt or solvate thereof, for 7 days per month or less, suitably 7 consecutive days per month or less.

In one embodiment provided herein is a method for the treatment or prevention of D. immitis infection in a mammal selected from a human, a dog or a cat, wherein said method comprises administering to said mammal a compound of formula I, or a pharmaceutically acceptable salt or solvate thereof, for 5 days per month or less, suitably 5 consecutive days per month or less.

In one embodiment provided herein is a method for the treatment or prevention of D. immitis infection in a mammal selected from a human, a dog or a cat, wherein said method comprises administering to said mammal a compound of formula I, or a pharmaceutically acceptable salt or solvate thereof, for 1 or 2 days per month, suitably 1 or 2 consecutive days per month or less.

In one embodiment provided herein is a method for the treatment or prevention of D. immitis L3 or L4 infection in a mammal selected from a human, a dog or a cat, wherein said method comprises administering to said mammal a compound of formula I, or a pharmaceutically acceptable salt or solvate thereof, for 7 days per month or less, suitably 7 consecutive days per month or less.

In one embodiment provided herein is a method for the treatment or prevention of D. immitis L3 or L4 infection in a mammal selected from a human, a dog or a cat, wherein said method comprises administering to said mammal a compound of formula I, or a pharmaceutically acceptable salt or solvate thereof, for 5 days per month or less, suitably 5 consecutive days per month or less.

In one embodiment provided herein is a method for the treatment or prevention of D. immitis L3 or L4 infection in a mammal selected from a human, a dog or a cat, wherein said method comprises administering to said mammal a compound of formula I, or a pharmaceutically acceptable salt or solvate thereof, for 1 or 2 days per month, suitably 1 or 2 consecutive days per month or less.

In one embodiment provided herein is a method for the treatment or prevention of D. immitis L3 or L4 infection in a mammal selected from a human, a dog or a cat, wherein said method comprises administering to said mammal a compound of formula I, or a pharmaceutically acceptable salt or solvate thereof, for 1 or 2 days per month, suitably 1 or 2 consecutive days per month or less.

In one embodiment provided herein is a method for the treatment or prevention of D. immitis mf infection in a mammal selected from a human, a dog or a cat, wherein said method comprises administering to said mammal a compound of formula I, or a pharmaceutically acceptable salt or solvate thereof, for 7 days per month or less, suitably 7 consecutive days per month or less.

In one embodiment provided herein is a method for the treatment or prevention of D. immitis mf infection in a mammal selected from a human, a dog or a cat, wherein said method comprises administering to said mammal a compound of formula I, or a pharmaceutically acceptable salt or solvate thereof, for 5 days per month or less, suitably 5 consecutive days per month or less.

In one embodiment provided herein is a method for the treatment or prevention of D. immitis mf infection in a mammal selected from a human, a dog or a cat, wherein said method comprises administering to said mammal a compound of formula I, or a pharmaceutically acceptable salt or solvate thereof, for 1 or 2 days per month, suitably 1 or 2 consecutive days per month or less.

In one embodiment provided herein is a method for the treatment or prevention of D. immitis mf infection in a mammal selected from a human, a dog or a cat, wherein said method comprises administering to said mammal a compound of formula I, or a pharmaceutically acceptable salt or solvate thereof, for 1 or 2 days per month, suitably 1 or 2 consecutive days per month or less.

In one embodiment provided herein is a method for the prevention of an adult dirofilarial worm infection in a mammal, wherein said method comprises administering to said mammal a compound of formula I, or a pharmaceutically acceptable salt or solvate thereof.

In another embodiment provided herein is a method for the prevention of an adult D. immitis or D. repens worm infection in a mammal, wherein said method comprises administering to said mammal a compound of formula I, or a pharmaceutically acceptable salt or solvate thereof, suitably where the mammal is selected from a human, dog or cat.

In another embodiment provided herein is a method for the prevention of an adult D. immitis worm infection in a dog, wherein said method comprises administering to said mammal a compound of formula I, or a pharmaceutically acceptable salt or solvate thereof, suitably wherein the compound is for administration for 1 or 2 days per month, suitably 1 or 2 consecutive days per month or less.

In one embodiment of each of the aforementioned aspects, the compound is a compound of sub-formula Ia, or a pharmaceutically acceptable salt or solvate thereof:

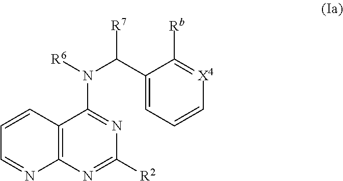

wherein,
R⁶ is selected from hydrogen and C₁₋₆ alkyl;
R⁷ is selected from hydrogen, ═O, C₃₋₆ cycloalkyl, C₁₋₆ alkyl, where said C₃₋₆ cycloalkyl, C₁₋₆ alkyl are optionally substituted by one or more R^{a}; or
R⁶ and R⁷, together with the atoms to which they are attached form a 3-7 membered heterocyclic ring, optionally substituted with one or more R^{a};
X⁴ is selected from CH and N;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1}, —OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1}, C₁₋₁₀haloalkyl, C₁₋₁₀alkyl optionally substituted by 1-13 R^{e}, C₂₋₆alkenyl optionally substituted by 1-11 R^{e}, C₂₋₆alkynyl optionally substituted by 1-9 R^{e}, C₆₋₁₁aryl optionally substituted by 1-11 R^{e}, (C₇₋₁₆)alkylaryl optionally substituted by 1-9 R^{e}, C₃₋₁₁cycloalkyl optionally substituted by 1-21 R^{e}, (C₄₋₁₇)cycloalkylalkyl optionally substituted by 1-32 R^{e}, 3-15 membered heterocycloalkyl optionally substituted by 1-28 R^{e}, 4-21 membered alkylheterocycloalkyl optionally substituted by 1-40 R^{e}, 5-15 membered heteroaryl optionally substituted by 1-15 R^{e}, and 6-21 membered alkylheteroaryl optionally substituted by 1-27 R^{e};
each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl, 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆ alkyl;
each R^{b} and R^{e} is independently selected from hydroxyl, ═O, halogen, CN, C₁₋₆ haloalkyl, C₁₋₆ haloalkoxy, C₃₋₆ cycloalkyl, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl; and
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl and O—C₁₋₆ alkyl, C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl; or
R^{c2} and R^{d2}, when attached to the same atom, together with the atom to which they are attached form a 3-7 membered ring, optionally substituted with one or more R^{a}.

In another embodiment, the compound of formula I is selected from one of the following compounds, or a pharmaceutically acceptable salt or solvate thereof:

N²-isopropyl-N²,N⁴-dimethyl-N⁴-(2-(trifluoromethyl)pyridin-3-yl)methyl)pyrido
[2,3-d]pyrimidine-2,4-diamine;
N²-isopropyl-N²-methyl-N⁴-(2-(trifluoromethyl)pyridin-3-yl)methyl)pyrido
[2,3-d]pyrimidine-2,4-diamine;
N²-isopropyl-N²-methyl-N⁴-((2-(trifluoromethyl)pyridin-3-yl)methyl)pyrido
[2,3-d]pyrimidine-2,4-diamine methanesulfonate;
2-(azetidin-1-yl)-N-((2-(trifluoromethyl)pyridin-3-yl)methyl)pyrido[2,3-d]pyrimidin-4-
amine;
N⁴-(2-fluoro-6-(trifluoromethyl)benzyl)-N²,N²-dimethylpyrido[2,3-d]pyrimidine-2,4-
diamine;
3-methyl-1-(4-(((2-(trifluoromethyl)pyridin-3-yl)methyl)amino)pyrido[2,3-d]pyrimidin-
2-yl)azetidin-3-ol;
2-(2-methylazetidin-1-yl)-N-((2-(trifluoromethyl)pyridin-3-yl)methyl)pyrido[2,3-d]
pyrimidin-4-amine;
2-(2,2-dimethylazetidin-1-yl)-N-((2-(trifluoromethyl)pyridin-3-yl)methyl)pyrido
[2,3-d]pyrimidin-4-amine;
2-(pyrrolidin-1-yl)-N-((2-(trifluoromethyl)pyridin-3-yl)methyl)pyrido[2,3-d]pyrimidin-4-
amine;
N²-cyclopropyl-N²-methyl-N⁴-((2-(trifluoromethyl)pyridin-3-yl)methyl)pyrido[2,3-d]
pyrimidine-2,4-diamine;
N-isopropyl-N-methyl-4-(2-(2-(trifluoromethyl)phenyl)azetidin-1-yl)pyrido[2,3-d]
pyrimidin-2-amine;
N-isopropyl-N-methyl-4-(2-(2-(trifluoromethyl)phenyl)piperidin-1-yl)pyrido[2,3-d]
pyrimidin-2-amine;
N-isopropyl-N-methyl-4-(3-(2-(trifluoromethyl)phenyl)morpholino)pyrido[2,3-d]
pyrimidin-2-amine;
N-isopropyl-N-methyl-4-(2-(2-(trifluoromethyl)phenyl)pyrazolidin-1-yl)pyrido[2,3-d]
pyrimidin-2-amine;
2-morpholino-N-((2-(trifluoromethyl)pyridin-3-yl)methyl)pyrido[2,3-d]pyrimidin-4-amine;
2-(3-methylmorpholino)-N-((2-(trifluoromethyl)pyridin-3-yl)methyl)pyrido[2,3-d]
pyrimidin-4-amine;
2-(3-methylmorpholino)-N-((2-(trifluoromethyl)pyridin-3-yl)methyl)pyrido[2,3-d]
pyrimidin-4-amine methanesulfonate;
2-(3,3-difluoropyrrolidin-1-yl)-N-((2-(trifluoromethyl)pyridin-3-yl)methyl)pyrido[2,3d]-
pyrimidin-4-amine;
2-(3,3-difluoropyrrolidin-1-yl)-N-((2-(trifluoromethyl)pyridin-3-yl)methyl)pyrido[2,3-d]
pyrimidin-4-amine methanesulfonate;
2-(4-fluoropiperidin-1-yl)-N-((2-(trifluoromethyl)pyridin-3-yl)methyl)pyrido[2,3-d]
pyrimidin-4-amine;
2-(4,4-difluoropiperidin-1-yl)-N-(2-(trifluoromethyl)benzyl)pyrido[2,3-d]pyrimidin-4-
amine;
2-(4,4-difluoropiperidin-1-yl)-N-((2-(trifluoromethyl)pyridin-3-yl)methyl)pyrido[2,3-d]
pyrimidin-4-amine;
2-(4-chloropiperidin-1-yl)-N-(2-(trifluoromethyl)benzyl)pyrido[2,3-d]pyrimidin-4-amine;
2-(4-chloropiperidin-1-yl)-N-((2-(trifluoromethyl)pyridin-3-yl)methyl)pyrido[2,3-d]
pyrimidin-4-amine;
2-(3-fluoroazetidin-1-yl)-N-((2-(trifluoromethyl)pyridin-3-yl)methyl)pyrido[2,3-d]
pyrimidin-4-amine;
2-(3,3-difluoroazetidin-1-yl)-N-((2-(trifluoromethyl)pyridin-3-yl)methyl)pyrido[2,3-d]
pyrimidin-4-amine;
2-(3-(trifluoromethyl)azetidin-1-yl)-N-((2-(trifluoromethyl)pyridin-3-yl)methyl)pyrido
[2,3-d]pyrimidin-4-amine;
(S)-2-(3-methylmorpholino)-N-(2-(trifluoromethyl)benzyl)pyrido[2,3-d]pyrimidin-4-
amine;
(S)-2-(3-methylmorpholino)-N-(2-(trifluoromethyl)benzyl)pyrido[2,3-d]pyrimidin-4-amine
methanesulfonate;
(R)-2-(3-methylmorpholino)-N-(2-(trifluoromethyl)benzyl)pyrido[2,3-d]pyrimidin-4-
amine;
(S)-2-(3-methylmorpholino)-N-((2-(trifluoromethyl)pyridin-3-yl)methyl)pyrido[2,3-d]
pyrimidin-4-amine;
(R)-2-(3-methylmorpholino)-N-((2-(trifluoromethyl)pyridin-3-yl)methyl)pyrido[2,3-d]
pyrimidin-4-amine;
2-(4-chloropiperidin-1-yl)-4-(2-(2-(trifluoromethyl)phenyl)azetidin-1-yl)pyrido[2,3-d]
pyrimidine;
2-(4,4-difluoropiperidin-1-yl)-4-(2-(2-(trifluoromethyl)phenyl)azetidin-1-yl)pyrido
[2,3-d]pyrimidine;
3-methyl-4-(4-(2-(2-(trifluoromethyl)phenyl)azetidin-1-yl)pyrido[2,3-d]pyrimidin-2-yl)
morpholine;
2-(3,3-difluoropyrrolidin-1-yl)-4-(2-(2-(trifluoromethyl)phenyl)azetidin-1-yl)pyrido
[2,3-d]pyrimidine;
2-(3-(trifluoromethyl)azetidin-1-yl)-4-(2-(2-(trifluoromethyl)phenyl)azetidin-1-yl)
pyrido[2,3-d]pyrimidine;
2-(4-chloropiperidin-1-yl)-4-(2-(2-(trifluoromethyl)pyridin-3-yl)azetidin-1-yl)pyrido
[2,3-d]pyrimidine;
2-(4,4-difluoropiperidin-1-yl)-4-(2-(2-(trifluoromethyl)pyridin-3-yl)azetidin-1-yl)
pyrido[2,3-d]pyrimidine;
3-methyl-4-(4-(2-(2-(trifluoromethyl)pyridin-3-yl)azetidin-1-yl)pyrido[2,3-d]pyrimidin-2-
yl)morpholine;
2-(3,3-difluoropyrrolidin-1-yl)-4-(2-(2-(trifluoromethyl)pyridin-3-yl)azetidin-1-yl)
pyrido[2,3-d]pyrimidine;
2-(3-(trifluoromethyl)azetidin-1-yl)-4-(2-(2-(trifluoromethyl)pyridin-3-yl)azetidin-1-yl)
pyrido[2,3-d]pyrimidine;
2-((4-chloropiperidin-1-yl)methyl)-N-((2-(trifluoromethyl)pyridin-3-yl)methyl)pyrido
[2,3-d]pyrimidin-4-amine;
2-((4,4-difluoropiperidin-1-yl)methyl)-N-((2-(trifluoromethyl)pyridin-3-yl)
methyl)pyrido[2,3-d]pyrimidin-4-amine;
2-((3-methylmorpholino)methyl)-N-((2-(trifluoromethyl)pyridin-3-yl)methyl)pyrido
[2,3-d]pyrimidin-4-amine;
2-((3,3-difluoropyrrolidin-1-yl)methyl)-N-((2-(trifluoromethyl)pyridin-3-yl)
methyl)pyrido[2,3-d]pyrimidin-4-amine;
2-((3-(trifluoromethyl)azetidin-1-yl)methyl)-N-((2-(trifluoromethyl)pyridin-3-yl)
methyl)pyrido[2,3-d]pyrimidin-4-amine;
2-((4-chloropiperidin-1-yl)methyl)-N-((2-(trifluoromethyl)pyridin-3-yl)methyl)pyrido
[2,3-d]pyrimidin-4-amine;
2-((4,4-difluoropiperidin-1-yl)methyl)-N-((2-(trifluoromethyl)pyridin-3-yl)
methyl)pyrido[2,3-d]pyrimidin-4-amine;
2-((3-methylmorpholino)methyl)-N-((2-(trifluoromethyl)pyridin-3-yl)methyl)pyrido
[2,3-d]pyrimidin-4-amine;
2-((3,3-difluoropyrrolidin-1-yl)methyl)-N-((2-(trifluoromethyl)pyridin-3-yl)
methyl)pyrido[2,3-d]pyrimidin-4-amine;
2-((3-(trifluoromethyl)azetidin-1-yl)methyl)-N-((2-(trifluoromethyl)pyridin-3-yl)
methyl)pyrido[2,3-d]pyrimidin-4-amine;
2-(4-(methylsulfonyl)piperidin-1-yl)-N-((2-(trifluoromethyl)pyridin-3-yl)methyl)pyrido
[2,3-d]pyrimidin-4-amine;
2-(4,4-dimethylpiperidin-1-yl)-N-((2-(trifluoromethyl)pyridin-3-yl)methyl)pyrido
[2,3-d]pyrimidin-4-amine;
1-(4-(((2-(trifluoromethyl)pyridin-3-yl)methyl)amino)pyrido[2,3-d]pyrimidin-2-
yl)piperidine-4-carbonitrile;
2-(4-(trifluoromethyl)piperidin-1-yl)-N-((2-(trifluoromethyl)pyridin-3-yl)methyl)pyrido
[2,3-d]pyrimidin-4-amine;
N-((2-(trifluoromethyl)pyridin-3-yl)methyl)-2-(3-(trifluoromethyl)pyrrolidin-1-yl)
pyrido[2,3-d]pyrimidin-4-amine;
1-(4-(((2-(trifluoromethyl)pyridin-3-yl)methyl)amino)pyrido[2,3-d]pyrimidin-2-yl)
azetidine-3-carbonitrile;
1-(4-(((2-(trifluoromethyl)pyridin-3-yl)methyl)amino)pyrido[2,3-d]pyrimidin-2-yl)
pyrrolidine-2-carbonitrile;
2-(2,2-dimethylmorpholino)-N-((2-(trifluoromethyl)pyridin-3-yl)methyl)pyrido
[2,3-d]pyrimidin-4-amine;
2-(3,3-dimethylmorpholino)-N-((2-(trifluoromethyl)pyridin-3-yl)methyl)pyrido
[2,3-d]pyrimidin-4-amine;
2-(2-methylmorpholino)-N-((2-(trifluoromethyl)pyridin-3-yl)methyl)pyrido[2,3-d]
pyrimidin-4-amine;
2-(3-fluoropyrrolidin-1-yl)-N-((2-(trifluoromethyl)pyridin-3-yl)methyl)pyrido[2,3-d]
pyrimidin-4-amine;
2-(4-(methylsulfonyl)piperidin-1-yl)-N-(2-(trifluoromethyl)benzyl)pyrido[2,3-d]pyrimidin-
4-amine;
2-(4,4-dimethylpiperidin-1-yl)-N-(2-(trifluoromethyl)benzyl)pyrido[2,3-d]pyrimidin-4-
amine;
1-(4-((2-(trifluoromethyl)benzyl)amino)pyrido[2,3-d]pyrimidin-2-yl)azetidine-3-
carbonitrile;
2-(3,3-dimethylmorpholino)-N-(2-(trifluoromethyl)benzyl)pyrido[2,3-d]pyrimidin-4-
amine;
2-(2,2-dimethylmorpholino)-N-(2-(trifluoromethyl)benzyl)pyrido[2,3-d]pyrimidin-4-
amine;
2-(2-methylmorpholino)-N-(2-(trifluoromethyl)benzyl)pyrido[2,3-d]pyrimidin-4-amine;
2-(3-(trifluoromethyl)piperidin-1-yl)-N-((2-(trifluoromethyl)pyridin-3-yl)methyl)pyrido
[2,3-d]pyrimidin-4-amine;
4-methyl-1-(4-(((2-(trifluoromethyl)pyridin-3-yl)methyl)amino)pyrido[2,3-d]pyrimidin-
2-yl)piperidine-4-carbonitrile;
1-(4-(((2-(trifluoromethyl)pyridin-3-yl)methyl)amino)pyrido[2,3-d]pyrimidin-2-yl)
piperidine-3-carbonitrile;
2-(2,2-difluoro-7-azaspiro[3.5]nonan-7-yl)-N-((2-(trifluoromethyl)pyridin-3-yl)
methyl)pyrido[2,3-d]pyrimidin-4-amine;
2-(2,2-difluoro-7-azaspiro[3.5]nonan-7-yl)-N-((2-(trifluoromethyl)pyridin-3-yl)
methyl)pyrido[2,3-d]pyrimidin-4-amine methanesulfonate;
2-(3-cyclopropylmorpholino)-N-((2-(trifluoromethyl)pyridin-3-yl)methyl)pyrido
[2,3-d]pyrimidin-4-amine;
2-((6S)-2,6-dimethylmorpholino)-N-((2-(trifluoromethyl)pyridin-3-yl)methyl)pyrido
[2,3-d]pyrimidin-4-amine;
2-(2-oxa-5-azabicyclo[2.2.1]heptan-5-yl)-N-((2-(trifluoromethyl)pyridin-3-yl)
methyl)pyrido[2,3-d]pyrimidin-4-amine;
2-(4-oxa-7-azaspiro[2.5]octan-7-yl)-N-((2-(trifluoromethyl)pyridin-3-yl)
methyl)pyrido[2,3-d]pyrimidin-4-amine;
4-(4-(((2-(trifluoromethyl)pyridin-3-yl)methyl)amino)pyrido[2,3-d]pyrimidin-2-yl)
morpholine-2-carbonitrile;
2-(3-(fluoromethyl)piperidin-1-yl)-N-((2-(trifluoromethyl)pyridin-3-yl)methyl)pyrido
[2,3-d]pyrimidin-4-amine;
2-(2-(trifluoromethyl)piperidin-1-yl)-N-((2-(trifluoromethyl)pyridin-3-yl)methyl)pyrido
[2,3-d]pyrimidin-4-amine;
2-(2,2-difluoro-7-azaspiro[3.5]nonan-7-yl)-N-(2-(trifluoromethyl)benzyl)pyrido[2,3-d]
pyrimidin-4-amine;
4-methyl-1-(4-((2-(trifluoromethyl)benzyl)amino)pyrido[2,3-d]pyrimidin-2-yl)piperidine-
4-carbonitrile;
1-(4-((2-(trifluoromethyl)benzyl)amino)pyrido[2,3-d]pyrimidin-2-yl)piperidine-3-
carbonitrile;
2-(3-(methylsulfonyl)pyrrolidin-1-yl)-N-((2-(trifluoromethyl)pyridin-3-yl)
methyl)pyrido[2,3-d]pyrimidin-4-amine;
N-((2-(trifluoromethyl)pyridin-3-yl)methyl)-2-(2-(trifluoromethyl)pyrrolidin-1-yl)
pyrido[2,3-d]pyrimidin-4-amine;
2-(3-chloropyrrolidin-1-yl)-N-((2-(trifluoromethyl)pyridin-3-yl)methyl)pyrido[2,3-d]
pyrimidin-4-amine;
N-(2-(trifluoromethyl)benzyl)-2-(3-(trifluoromethyl)pyrrolidin-1-yl)pyrido[2,3-d]pyrimidin-
4-amine;
2-(2-oxa-5-azabicyclo[2.2.1]heptan-5-yl)-N-(2-(trifluoromethyl)benzyl)pyrido[2,3-d]
pyrimidin-4-amine;
2-((6S)-2,6-dimethylmorpholino)-N-(2-(trifluoromethyl)benzyl)pyrido[2,3-d]pyrimidin-4-
amine;
2-(3-isopropylmorpholino)-N-((2-(trifluoromethyl)pyridin-3-yl)methyl)pyrido[2,3-d]
pyrimidin-4-amine;
2-((2R,3R)-2,3-dimethylmorpholino)-N-((2-(trifluoromethyl)pyridin-3-yl)
methyl)pyrido[2,3-d]pyrimidin-4-amine;
2-((2S,5R)-2,5-dimethylmorpholino)-N-((2-(trifluoromethyl)pyridin-3-yl)
methyl)pyrido[2,3-d]pyrimidin-4-amine;
2-(6-oxa-9-azaspiro[4.5]decan-9-yl)-N-((2-(trifluoromethyl)pyridin-3-yl)
methyl)pyrido[2,3-d]pyrimidin-4-amine;
2-(4-(trifluoromethoxy)piperidin-1-yl)-N-((2-(trifluoromethyl)pyridin-3-yl)
methyl)pyrido[2,3-d]pyrimidin-4-amine;
2-(4-(trifluoromethoxy)piperidin-1-yl)-N-((2-(trifluoromethyl)pyridin-3-yl)
methyl)pyrido[2,3-d]pyrimidin-4-amine methanesulfonate;
2-(3-(difluoromethyl)azetidin-1-yl)-N-((2-(trifluoromethyl)pyridin-3-yl)methyl)pyrido
[2,3-d]pyrimidin-4-amine;
2-((3R)-3,5-dimethylmorpholino)-N-((2-(trifluoromethyl)pyridin-3-yl)methyl)pyrido
[2,3-d]pyrimidin-4-amine;
2-(3-(difluoromethyl)azetidin-1-yl)-N-(2-(trifluoromethyl)benzyl)pyrido[2,3-d]pyrimidin-4-
amine;
2-((3R)-3,5-dimethylmorpholino)-N-(2-(trifluoromethyl)benzyl)pyrido[2,3-d]pyrimidin-4-
amine;
2-(2-cyclopropylmorpholino)-N-((2-(trifluoromethyl)pyridin-3-yl)methyl)pyrido[2,3-d]
pyrimidin-4-amine;
2-((2S,5S)-2,5-dimethylmorpholino)-N-((2-(trifluoromethyl)pyridin-3-yl)
methyl)pyrido[2,3-d]pyrimidin-4-amine;
(R)-2-(3-(difluoromethoxy)pyrrolidin-1-yl)-N-((2-(trifluoromethyl)pyridin-3-yl)
methyl)pyrido[2,3-d]pyrimidin-4-amine;
(S)-2-(3-(difluoromethoxy)pyrrolidin-1-yl)-N-((2-(trifluoromethyl)pyridin-3-yl)
methyl)pyrido[2,3-d]pyrimidin-4-amine;
2-(2-(difluoromethyl)morpholino)-N-((2-(trifluoromethyl)pyridin-3-yl)methyl)pyrido
[2,3-d]pyrimidin-4-amine;
4-(4-((2-(trifluoromethyl)benzyl)amino)pyrido[2,3-d]pyrimidin-2-yl)morpholine-2-
carbonitrile;
2-(2-(difluoromethyl)morpholino)-N-(2-(trifluoromethyl)benzyl)pyrido[2,3-d]pyrimidin-4-
amine;
2-(8-oxa-5-azaspiro[3.5]nonan-5-yl)-N-(2-(trifluoromethyl)benzyl)pyrido[2,3-d]
pyrimidin-4-amine;
2-(9-oxa-6-azaspiro[4.5]decan-6-yl)-N-(2-(trifluoromethyl)benzyl)pyrido[2,3-d]
pyrimidin-4-amine;
2-((2R,3S)-2,3-dimethylmorpholino)-N-((2-(trifluoromethyl)pyridin-3-yl)
methyl)pyrido[2,3-d]pyrimidin-4-amine;
(3R)-3-methyl-4-(4-(2-(2-(trifluoromethyl)pyridin-3-yl)azetidin-1-yl)pyrido[2,3-d]
pyrimidin-2-yl)morpholine;
(3S)-3-methyl-4-(4-(2-(2-(trifluoromethyl)pyridin-3-yl)azetidin-1-yl)pyrido[2,3-d]
pyrimidin-2-yl)morpholine;
(3R)-3-methyl-4-(4-(2-(2-(trifluoromethyl)phenyl)azetidin-1-yl)pyrido[2,3-d]pyrimidin-
2-yl)morpholine;
(3S)-3-methyl-4-(4-(2-(2-(trifluoromethyl)phenyl)azetidin-1-yl)pyrido[2,3-d]pyrimidin-
2-yl)morpholine;
2-(3-(difluoromethoxy)piperidin-1-yl)-N-((2-(trifluoromethyl)pyridin-3-yl)
methyl)pyrido[2,3-d]pyrimidin-4-amine;
2-(6-oxa-3-azabicyclo[3.1.1]heptan-3-yl)-N-((2-(trifluoromethyl)pyridin-3-yl)
methyl)pyrido[2,3-d]pyrimidin-4-amine;
2-(2-oxa-5-azabicyclo[4.1.0]heptan-5-yl)-N-((2-(trifluoromethyl)pyridin-3-yl)
methyl)pyrido[2,3-d]pyrimidin-4-amine;
2-(2,2,6,6-tetrafluoromorpholino)-N-((2-(trifluoromethyl)pyridin-3-yl)methyl)pyrido
[2,3-d]pyrimidin-4-amine;
2-(4-azaspiro[2.5]octan-4-yl)-N-((2-(trifluoromethyl)pyridin-3-yl)methyl)pyrido
[2,3-d]pyrimidin-4-amine;
2-(3-(trifluoromethoxy)pyrrolidin-1-yl)-N-((2-(trifluoromethyl)pyridin-3-yl)
methyl)pyrido[2,3-d]pyrimidin-4-amine;
2-(5-azaspiro[3.4]octan-5-yl)-N-((2-(trifluoromethyl)pyridin-3-yl)methyl)pyrido
[2,3-d]pyrimidin-4-amine;
2-(2-((trifluoromethoxy)methyl)pyrrolidin-1-yl)-N-((2-(trifluoromethyl)pyridin-3-yl)
methyl)pyrido[2,3-d]pyrimidin-4-amine;
2-(6-oxa-3-azabicyclo[3.1.1]heptan-3-yl)-N-(2-(trifluoromethyl)benzyl)pyrido[2,3-d]
pyrimidin-4-amine;
2-(2-oxa-5-azabicyclo[4.1.0]heptan-5-yl)-N-(2-(trifluoromethyl)benzyl)pyrido[2,3-d]
pyrimidin-4-amine;
2-((3R)-3,5-dimethylmorpholino)-N-(2-(trifluoromethyl)benzyl)pyrido[2,3-d]pyrimidin-4-
amine;
6-fluoro-2-(3-methylmorpholino)-N-((2-(trifluoromethyl)pyridin-3-yl)methyl)pyrido
[2,3-d]pyrimidin-4-amine;
6-methoxy-2-(3-methylmorpholino)-N-((2-(trifluoromethyl)pyridin-3-yl)
methyl)pyrido[2,3-d]pyrimidin-4-amine;
7-methoxy-2-(3-methylmorpholino)-N-((2-(trifluoromethyl)pyridin-3-yl)
methyl)pyrido[2,3-d]pyrimidin-4-amine;
5-methoxy-2-(3-methylmorpholino)-N-((2-(trifluoromethyl)pyridin-3-yl)
methyl)pyrido[2,3-d]pyrimidin-4-amine;
6,7-dimethoxy-2-(3-methylmorpholino)-N-((2-(trifluoromethyl)pyridin-3-yl)
methyl)pyrido[2,3-d]pyrimidin-4-amine; and
2-(3-methylmorpholino)-7-(2-morpholinoethoxy)-N-((2-(trifluoromethyl)pyridin-3-yl)
methyl)pyrido[2,3-d]pyrimidin-4-amine.

In another embodiment, the compound of formula I is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2′3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In another embodiment, the compound of formula I is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In another embodiment, the compound of formula I is 2-(3-methylmorpholino)-N-((2-(trifluoromethyl)pyridin-3-yl)methyl)pyrido[2,3-d]pyrimidin-4-amine or a pharmaceutically acceptable salt or solvate thereof. Suitably, the compound is (S)-2-(3-methylmorpholino)-N-((2-(trifluoromethyl)pyridin-3-yl)methyl)pyrido[2,3-d]pyrimidin-4-amine or a pharmaceutically acceptable salt or solvate thereof.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis or D. repens infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or a pharmaceutically acceptable salt or solvate thereof is for administration for 7 days per month or less, suitably 7 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis or D. repens infection in a mammal selected from a human, a dog or a cat, wherein the compound, or a pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis or D. repens infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis or D. repens infection in a mammal selected from a human, a dog or a cat, wherein the compound, or a pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis or D. repens infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis or D. repens infection in a mammal selected from a human, a dog or a cat, wherein the compound, or pharmaceutically acceptable salt or solvate thereof is for administration for 1 or 2 days per month, suitably 1 or 2 consecutive days per month. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis infection in a mammal selected from a human, a dog or a cat, wherein the compound, or pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis infection in a mammal selected from a human, a dog or a cat, wherein the compound, or pharmaceutically acceptable salt or solvate thereof is for administration for 5 days per month or less, suitably 5 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less.

In one embodiment provided herein is a compound selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis L3 or L4 infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of D. immitis mf infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 7 days per month or less, suitably 7 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the treatment or prevention of adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the treatment or prevention of adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 5 days per month or less, suitably 5 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is 2-(3-methylmorpholino)-N-((2-(trifluoromethyl)pyridin-3-yl)methyl)pyrido[2,3-d]pyrimidin-4-amine, or pharmaceutically acceptable salt or solvate thereof, for use in the prevention of an adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less.

In one embodiment provided herein is 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof, for use in the prevention of an adult D. immitis infection in a mammal selected from a human, a dog or a cat, wherein 2-(3-methylmorpholino)-N-((2-(trifluoromethyl)pyridin-3-yl)methyl)pyrido[2,3-d]pyrimidin-4-amine, or pharmaceutically acceptable salt or solvate thereof is for administration for 1 or 2 days per month, suitably 1 or 2 consecutive days per month or less. Suitably, the compound is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof. Suitably, the compound 2-(3-methylmorpholino)-N-((2-(trifluoromethyl)pyridin-3-yl)methyl)pyrido[2,3-d]pyrimidin-4-amine is (S)-2-(3-methylmorpholino)-N-((2-(trifluoromethyl)pyridin-3-yl)methyl)pyrido[2,3-d]pyrimidin-4-amine.

In one embodiment provided herein is a method for the treatment or prevention of D. immitis or D. repens infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof for 7 days per month or less, suitably 7 consecutive days per month or less.

In one embodiment provided herein is a method for the treatment or prevention of D. immitis or D. repens infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof for 5 days per month or less, suitably 5 consecutive days per month or less.

In one embodiment provided herein is a method for the treatment or prevention of D. immitis or D. repens infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof for administration for 1 or 2 days per month, suitably 1 or 2 consecutive days per month.

In one embodiment provided herein is a method for the treatment or prevention of D. immitis infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 7 days per month or less, suitably 7 consecutive days per month or less.

In one embodiment provided herein is a method for the treatment or prevention of D. immitis infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 5 days per month or less, suitably 5 consecutive days per month or less.

In one embodiment provided herein is a method for the treatment or prevention of D. immitis infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In one embodiment provided herein is a method for the treatment or prevention of D. immitis L3 or L4 infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 7 days per month or less, suitably 7 consecutive days per month or less.

In one embodiment provided herein is a method for the treatment or prevention of D. immitis L3 or L4 infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 5 days per month or less, suitably 5 consecutive days per month or less.

In one embodiment provided herein is a method for the treatment or prevention of D. immitis L3 or L4 infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In one embodiment provided herein is a method for the treatment or prevention of D. immitis L3 or L4 infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In one embodiment provided herein is a method for the treatment or prevention of D. immitis mf infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 7 days per month or less, suitably 7 consecutive days per month or less.

In one embodiment provided herein is a method for the treatment or prevention of D. immitis mf infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 5 days per month or less, suitably 5 consecutive days per month or less.

In one embodiment provided herein is a method for the treatment or prevention of D. immitis mf infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In one embodiment provided herein is a method for the treatment or prevention of D. immitis mf infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In one embodiment provided herein is a method for prevention of an adult D. immitis infection in a mammal selected from a human, a dog or a cat, wherein said method comprises administering to said mammal 2-(3-methylmorpholino)-N-((2-(trifluoromethyl)pyridin-3-yl)methyl)pyrido[2,3-d]pyrimidin-4-amine, or a pharmaceutically acceptable salt or solvate thereof, for 1 or 2 days per month, suitably 1 or 2 consecutive days per month or less.

In another aspect, the present invention provides a compound of formula I as defined herein, or a pharmaceutically acceptable salt or solvate thereof, for use in the treatment or prevention of a disease or condition caused by a Dirofilaria infection in a mammal.

In another aspect, the present invention provides a method of treating or preventing a disease or condition caused by a Dirofilaria infection in a mammal comprising administering to said mammal an effective amount of a compound of formula I as defined herein, or a pharmaceutically acceptable salt or solvate thereof.

In another aspect, the present invention provides the use of a compound of formula I as defined herein, or a pharmaceutically acceptable salt or solvate thereof, for treating or preventing a disease or condition caused by a Dirofilaria infection in a mammal.

In another aspect, the present invention provides the use of a compound of formula I as defined herein, or a pharmaceutically acceptable salt or solvate thereof, in the manufacture of medicament for the treatment or prevention of a disease or condition caused by a Dirofilaria infection in a mammal.

In one embodiment, the disease or condition caused by the Dirofilaria infection is selected from heartworm disease (HWD), heartworm associated respiratory disease (HARD), or a dermatological condition.

In one embodiment, the dermatological condition is selected from pruritis, erythema, alopecia and eczema.

In one embodiment, the Dirofilaria infection is D. immitis infection and the disease or condition is heartworm disease (HWD), heartworm associated respiratory disease (HARD). Accordingly, in one embodiment, the present invention relates to a compound of formula I as defined herein, or a pharmaceutically acceptable salt or solvate thereof, for use in the treatment or prevention of heartworm disease (HWD) or heartworm associated respiratory disease (HARD) in a mammal. In another embodiment, the present invention relates to a method of treating or preventing heartworm disease (HWD) or heartworm associated respiratory disease (HARD) in a mammal comprising administering to said mammal an effective amount of a compound of formula I as defined herein, or a pharmaceutically acceptable salt or solvate thereof.

In one embodiment, the Dirofilaria infection is D. repens infection and the disease or condition a dermatological condition, suitably selected from one or more of pruritis, erythema, alopecia and eczema. Accordingly, in one embodiment, the present invention relates to a compound of formula I as defined herein, or a pharmaceutically acceptable salt or solvate thereof, for use in the treatment or prevention of a dermatological condition, suitably selected from one or more of pruritis, erythema, alopecia and eczema, in a mammal. In another embodiment, the present invention relates to a method of treating or preventing a dermatological condition, suitably selected from one or more of pruritis, erythema, alopecia and eczema in a mammal comprising administering to said mammal an effective amount of a compound of formula I as defined herein, or a pharmaceutically acceptable salt or solvate thereof.

In one embodiment, the mammal is a dog and the disease or condition is canine heartworm disease.

In one embodiment, the mammal is a cat and the disease or condition is heartworm associated respiratory disease.

The invention will now be further described by way of the following numbered paragraphs which are not claims:

1. A compound of formula I, or pharmaceutically acceptable salt or solvate thereof, for use in the treatment or prevention of a Dirofilaria infection in a mammal,

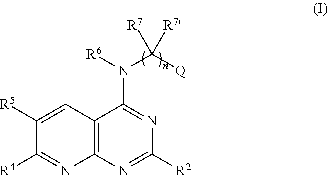

wherein
Q is a group selected from an C₃₋₁₁cycloalkyl optionally substituted by one or more R^{b}, 3-15 membered heterocycloalkyl optionally substituted by one or more R^{b}, C₆₋₁₁ aryl group optionally substituted with by one or more R^{b}, 5-15 membered heteroaryl optionally substituted by one or more R^{b};
R⁶ is selected from hydrogen and C₁₋₆ alkyl;
R⁷ and R^{7′} are independently selected from hydrogen, C₃₋₆ cycloalkyl, C₁₋₆ alkyl, where said C₃₋₆ cycloalkyl and C₁₋₆ alkyl are optionally substituted by one or more R^{a}; or
R⁷ and R^{7′}, together with the carbon to which they are attached form a 3-7 membered cycloalkyl ring, optionally substituted with one or more R^{a}, or R⁷ and R^{7′}, together with the carbon to which they are attached form a carbonyl group; or
R⁶ and R⁷, together with the atoms to which they are attached form a 3-7 membered heterocyclic ring, optionally substituted with one or more R^{a};
n is a number selected from 1, 2 and 3;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1} OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1} S(═O)₂NR^{c1}R^{d1}. C₁₋₁₀ haloalkyl, C₁₋₁₀alkyl optionally substituted by one or more R^{e}, C₂₋₆alkenyl optionally substituted by one or more R^{e}, C₂₋₆alkynyl optionally substituted by one or more R^{e}, C₆₋₁₁aryl optionally substituted by one or more R^{e}, (C₇₋₁₆)alkylaryl optionally substituted by one or more R^{e}, C₃₋₁₁cycloalkyl optionally substituted by one or more R^{e}, (C₄₋₁₇)cycloalkylalkyl optionally substituted by one or more R^{e}, 3-15 membered heterocycloalkyl optionally substituted by one or more R^{e}, 4-21 membered alkylheterocycloalkyl optionally substituted by one or more R^{e}, 5-15 membered heteroaryl optionally substituted by one or more R^{e}, and 6-21 membered alkylheteroaryl optionally substituted by one or more R^{e};
where each R^{c1} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d1} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c1} and R^{d1}, when attached to the same atom, together with the atom to which they are attached form a 3-7 membered ring, optionally containing one or more for heteroatoms selected from O, NH and S, and wherein said ring is optionally substituted with one or more R^{a};
where each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl and 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆alkyl;
each R^{b} and R^{e} is independently selected from hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c2} and R^{d2}, when attached to the same atom, together with the atom to which they are attached form a 3-7 membered ring, optionally containing one or more for heteroatoms selected from O, NH and S, and wherein said ring is optionally substituted with one or more R^{a};
R³, R⁴ and R⁵ are independently selected from hydrogen, hydroxyl, halogen, CN, C₁₋₆ haloalkyl, C₁₋₆ haloalkoxy, C₁₋₆ alkyl, phenyl and cyclopropyl, wherein said C₁₋₆ alkyl, phenyl and cyclopropyl are optionally substituted by one or more R^{a};

2. A compound of formula I, or a pharmaceutically acceptable salt or solvate thereof, for use according to paragraph 1 wherein the Dirofilaria infection is infection with one or more parasite selected from D. immitis, D. repens and D. tenuis.

3. A compound of formula I, or a pharmaceutically acceptable salt or solvate thereof, for use according to paragraph 1 or paragraph 2 wherein the Dirofilaria infection is infection with D. immitis.

4. A compound of formula I, or a pharmaceutically acceptable salt or solvate thereof, for use according to paragraph 1 or paragraph 2 wherein the Dirofilaria infection is infection with drug resistant D. immitis.

5. A compound of formula I, or a pharmaceutically acceptable salt or solvate thereof, for use according to any one of the preceding paragraphs wherein the Dirofilaria infection is infection with Dirofilaria microfilariae, Dirofilaria third stage (L3) larvae, Dirofilaria fourth stage (L4) larvae, Dirofilaria fifth stage (L5) larvae, an adult dirofilarial worm or a combination thereof.

6. A compound of formula I, or a pharmaceutically acceptable salt or solvate thereof, for use according to any one of the preceding paragraphs wherein the Dirofilaria infection is infection with Dirofilaria L3 or L4 larvae.

7. A compound of formula I, or a pharmaceutically acceptable salt or solvate thereof, for use according to any one of paragraphs 1 to 3 wherein the Dirofilaria infection is infection with D. immitis microfilariae (mf), L3 larvae, L4 larvae or a combination thereof.

8. A compound of formula I, or a pharmaceutically acceptable salt or solvate thereof, for use according to any one of the preceding paragraphs wherein the mammal is selected from a human, a dog, a cat, a racoon, a coyote, a jackal, a wolf, a fox, a ferret.

9. A compound of formula I, or a pharmaceutically acceptable salt or solvate thereof, for use according to any one of the preceding paragraphs wherein the mammal is selected from a human, a dog and a cat.

10. A compound of formula I, or a pharmaceutically acceptable salt or solvate thereof, for use according to any one of the preceding paragraphs wherein the mammal is a dog.

11. A compound of formula I, or a pharmaceutically acceptable salt or solvate thereof, as defined in paragraph 1 for use in the treatment or prevention of a disease or condition caused by a Dirofilaria infection in a mammal.

12. A compound of formula I, or a pharmaceutically acceptable salt or solvate thereof, for use according to paragraph 11 wherein the disease or condition is selected from heartworm disease (HWD), heartworm associated respiratory disease (HARD) or a dermatological condition.

13. A compound of formula I, or a pharmaceutically acceptable salt or solvate thereof, for use according to paragraph 11 or 12 wherein the disease or condition is selected from heartworm disease (HWD) or a dermatological condition and the mammal is a dog.

14. A compound of formula I, or a pharmaceutically acceptable salt or solvate thereof, for use according to paragraph 11 or 12 wherein the disease or condition is selected from heartworm associated respiratory disease (HARD) and the mammal is a cat.

15. A compound of formula I, or a pharmaceutically acceptable salt or solvate thereof, for use according to any one of paragraphs 11 to 14 wherein the Dirofilaria infection is D. immitis infection.

16. A compound of formula I, or a pharmaceutically acceptable salt or solvate thereof, for use according to any one of the preceding paragraphs wherein the compound is for administration for 7 consecutive days per month or less.

17. A compound of formula I, or a pharmaceutically acceptable salt or solvate thereof, for use according to any one of the preceding paragraphs wherein the compound is for administration for 1 or 2 consecutive days per month or less.

18. A compound of formula I, or a pharmaceutically acceptable salt or solvate thereof, for use according to any one of the preceding paragraphs wherein the compound is for oral administration.

19. A method of treating or preventing a Dirofilaria infection in a mammal comprising administering to said mammal an effective amount of a compound of formula I as defined in paragraph 1, or a pharmaceutically acceptable salt or solvate thereof.

20. A method according to paragraph 19 wherein the Dirofilaria infection is infection with one or more parasite selected from D. immitis, D. repens and D. tenuis.

21. A method according to paragraph 19 or paragraph 20 wherein the Dirofilaria infection is infection with D. immitis.

22. A method according to paragraph 19 or paragraph 20 wherein the Dirofilaria infection is infection with drug resistant D. immitis.

23. A method according to any one of paragraphs 19 to 22 wherein the Dirofilaria infection is infection with Dirofilaria microfilariae, Dirofilaria third stage (L3) larvae, Dirofilaria fourth stage (L4) larvae, Dirofilaria fifth stage (L5) larvae, an adult dirofilarial worm or a combination thereof.

24. A method according to any one of paragraphs 19 to 23 wherein the Dirofilaria infection is infection with Dirofilaria L3 or L4 larvae.

25. A method according to any one of paragraphs 19 to 22 wherein the Dirofilaria infection is infection with D. immitis microfilariae (mf), L3 larvae, L4 larvae or a combination thereof.

26. A method according to any one of paragraphs 19 to 25 wherein the mammal is selected from a human, a dog, a cat, a racoon, a coyote, a jackal, a wolf, a fox, a ferret.

27. A method according to any one of paragraph 19 to 26 wherein the mammal is selected from a human, a dog and a cat.

28. A method according to any one of paragraph 19 to 27 wherein the mammal is a dog.

29. A method of treating or preventing a disease or condition caused by a Dirofilaria infection in a mammal comprising administering to said mammal an effective amount of a compound of formula I as defined in paragraph 1, or a pharmaceutically acceptable salt or solvate thereof.

30. A method according to paragraph 29 wherein the disease or condition is selected from heartworm disease (HWD), heartworm associated respiratory disease (HARD) or a dermatological condition.

31. A method according to paragraph 29 or 30 wherein the disease or condition is selected from heartworm disease (HWD) or a dermatological condition and the mammal is a dog.

32. A method according to paragraph 29 or 30 wherein the disease or condition is selected from heartworm associated respiratory disease (HARD) and the mammal is a cat.

33. A method according to any one of paragraphs 29 to 32 wherein the Dirofilaria infection is D. immitis infection.

34. A method according to any one of paragraphs 19 to 33 wherein the compound is for administration for 7 consecutive days per month or less.

35. A method according to any one of paragraphs 19 to 33 wherein the compound is for administration for 1 or 2 consecutive days per month or less.

36. A method according to any one of paragraph 19 to 33 wherein the compound is for oral administration.

37. A compound for use/a method according to any one of the preceding paragraphs wherein when Q is phenyl, R^{b} is not such that Q is a 3,4-di-O—C₁₋₆ alkyl phenyl, a 3,5-di-O—C₁₋₆ alkyl phenyl or a 3,4,5-tri-O—C₁₋₆ alkyl phenyl

38. A compound for use/a method according to any one of the preceding paragraphs wherein the compound of formula I is not N-(4-fluorobenzyl)-2-(piperidinyl-1-yl)pyrido[2,3-d]pyrimidin-4-amine.

40. A compound for use/a method according to any one of the preceding paragraphs, wherein Q is a group selected from a C₆₋₁₁ aryl group optionally substituted with by 1-11 R^{b} and a 5-15 membered heteroaryl optionally substituted by 1-15 R^{b}.

41. A compound for use/a method according to any one of the preceding paragraphs, wherein Q is selected from a phenyl or pyridyl group optionally substituted with 1-5 R^{b}.

42. A compound for use/a method according to any one of the preceding paragraphs, wherein Q is a group of Formula III:

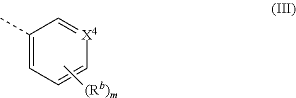

wherein
X⁴ is selected from CH and N;
m is selected from 0, 1 and 2; and
R^{b} is as previously defined.

43. A compound for use/a method according to any one of the preceding paragraphs, wherein Q is a group of Formula IIIa:

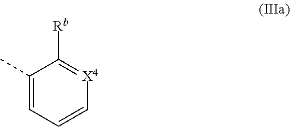

wherein
X⁴ is selected from CH and N; and
R^{b} is as previously defined.

44. A compound for use/a method according to any one of the preceding paragraphs, wherein each R^{b} is independently selected from hydroxyl, ═O, halogen, CN, C₁₋₆ haloalkyl, C₁₋₆ haloalkoxy, C₃₋₆ cycloalkyl, C₁₋₆ alkyl, O—C₁₋₆ alkyl, C₃₋₆ cycloalkyl, 3-7 membered heterocycloalkyl, —NR^{c2}R^{d2}, —NR^{c2}C(═O)R^{d2}, —OR^{d2}, —SR^{d2}, —S(═O)₂R^{d2}, —S(═O)R^{d2}, —S(═O)NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl.

45. A compound for use/a method according to any one of the preceding paragraphs, wherein each R^{b} is independently selected from fluoro, chloro, and CF₃.

46. A compound for use/a method according to any one of the preceding paragraphs, wherein R⁶, R⁷ and R^{7′} are each hydrogen.

47. A compound for use/a method according to any one of the preceding paragraphs, wherein n is 1.

48. A compound for use/a method according to any one of the preceding paragraphs, wherein R² is selected from —CN, —C(═O)R^{d1}, —C(═O)OR^{d1}, —C(═O)NR^{c1}R^{d1}, —NR^{c1}R^{d1}, —NR^{c1}C(═O)R^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1}, —OC(═O)R^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1} C₁₋₁₀ haloalkyl, C₁₋₁₀alkyl optionally substituted by 1-13 R^{e}, C₆₋₁₁aryl optionally substituted by 1-11 R^{e}, C₃₋₁₁cycloalkyl optionally substituted by 1-21 R^{e}, 3-15 membered heterocycloalkyl optionally substituted by 1-28 R^{e}, and 5-15 membered heteroaryl optionally substituted by 1-15 R^{e}.

49. A compound for use/a method according to any one of the preceding paragraphs, wherein R² is a 3-15 membered heterocycloalkyl optionally substituted by 1-28 R^{e}, or NR^{c1}R^{d1} wherein R^{c1} is C₁₋₆ alkyl, and R^{d1} is independently selected from C₃₋₆ cycloalkyl, C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl.

50. A compound for use/a method according to any one of the preceding paragraphs, wherein R² is a 3-7 membered heterocycloalkyl optionally substituted by 1-28 R^{e}, or NR^{c1}R^{d1} wherein R^{c1} is C₁₋₆ alkyl, and R^{d1} is independently selected from C₃₋₆ cycloalkyl, C₁₋₆ alkyl and C₆₋₁₁ aryl, wherein said C₁₋₆ alkyl, C₆₋₁₁ aryl, and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl.

51. A compound for use/a method according to any one of the preceding paragraphs, wherein R² is selected from

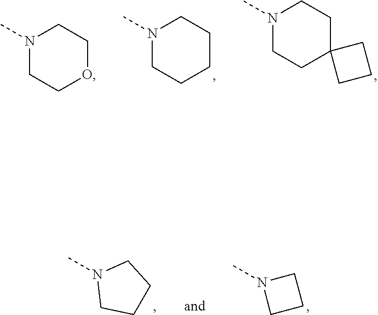

each of which may optionally be substituted with one or more R^{e}.

52. A compound for use/a method according to any one of the preceding paragraphs, wherein R^{e} is independently selected from hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, 3-7 membered heterocycloalkyl, —NR^{c2}R^{d2} where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl.

53. A compound for use/a method according to any one of the preceding paragraphs, wherein R^{e} is independently selected from hydroxyl, ═O, halogen, CN, C₁₋₆ haloalkyl, C₁₋₆ haloalkoxy, C₁₋₆ alkyl and O—C₁₋₆ alkyl.

54. A compound for use/a method according to any one of the preceding paragraphs, wherein each R^{e} is independently selected from fluoro, chloro, CN, CF₃, OCF₃, and methyl.

55. A compound for use/a method according to any one of the preceding paragraphs, wherein each R^{c1} or R^{c2} is independently selected from hydrogen and C₁₋₆ alkyl.

56. A compound for use/a method according to any one of the preceding paragraphs, wherein each R^{d1} or R^{d2} is independently selected from C₃₋₆ cycloalkyl, C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl.

57. A compound for use/a method according to any one of paragraphs 1 to 36, wherein the compound is of sub-formula Ia:

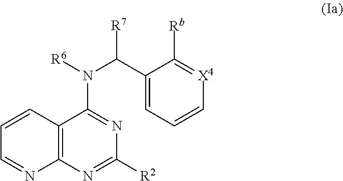

wherein,
R⁶ is selected from hydrogen and C₁₋₆ alkyl;
R⁷ is selected from hydrogen, ═O, C₃₋₆ cycloalkyl, C₁₋₆ alkyl, where said C₃₋₆ cycloalkyl, C₁₋₆ alkyl are optionally substituted by one or more R^{a}; or
R⁶ and R⁷, together with the atoms to which they are attached form a 3-7 membered heterocyclic ring, optionally substituted with one or more R^{a};
X⁴ is selected from CH and N;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1}, —OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1}, C₁₋₁₀haloalkyl, C₁₋₁₀alkyl optionally substituted by 1-13 R^{e}, C₂₋₆alkenyl optionally substituted by 1-11 R^{e}, C₂₋₆alkynyl optionally substituted by 1-9 R^{e}, C₆₋₁₁aryl optionally substituted by 1-11 R^{e}, (C₇₋₁₆)alkylaryl optionally substituted by 1-9 R^{e}, C₃₋₁₁cycloalkyl optionally substituted by 1-21 R^{e}, (C₄₋₁₇)cycloalkylalkyl optionally substituted by 1-32 R^{e}, 3-15 membered heterocycloalkyl optionally substituted by 1-28 R^{e}, 4-21 membered alkylheterocycloalkyl optionally substituted by 1-40 R^{e}, 5-15 membered heteroaryl optionally substituted by 1-15 R^{e}, and 6-21 membered alkylheteroaryl optionally substituted by 1-27 R^{e};
each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl, 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆ alkyl;
each R^{b} and R^{e} is independently selected from hydroxyl, ═O, halogen, CN, C₁₋₆ haloalkyl, C₁₋₆ haloalkoxy, C₃₋₆ cycloalkyl, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl; and
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl and O—C₁₋₆ alkyl, C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl; or
R^{c2} and R^{d2}, when attached to the same atom, together with the atom to which they are attached form a 3-7 membered ring, optionally substituted with one or more R^{a}.

58. A compound for use/a method according to paragraph 57, wherein each R^{b} is independently selected from fluoro, chloro, and CF₃.

59. A compound for use/a method according to paragraph 57 or paragraph 58, wherein R⁶ and R⁷ are both hydrogen.

60. A compound for use/a method according to any one of paragraphs 57 to 59, wherein R² is selected from —CN, —C(═O)R^{d1}, C(═O)NR^{c1}R^{d1}, —NR^{c1}R^{d1}, —NR^{c1}C(═O)NR^{c1}R^{d1}, —OR^{d1}, —SR^{d1}, S(═O)₂R^{d1}, C₁₋₁₀ haloalkyl, C₁₋₁₀alkyl optionally substituted by 1-13 R^{e}, C₆₋₁₁aryl optionally substituted by 1-11 R^{e}, C₃₋₁₁cycloalkyl optionally substituted by 1-21 R^{e}, 3-15 membered heterocycloalkyl optionally substituted by 1-28 R^{e}, and 5-15 membered heteroaryl optionally substituted by 1-15 R^{e}.

61. A compound for use/a method according to any one of paragraphs 57 to 60, wherein R² is selected from 3-8 membered heterocycloalkyl optionally substituted by one or more R^{e}.

62. A compound for use/a method according to any one of paragraphs 57 to 61, wherein R² is selected from

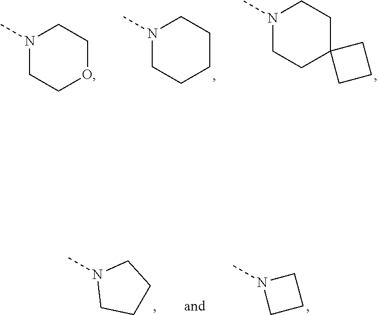

each of which may optionally be substituted with one or more R^{e}.

63. A compound for use/a method according to any one of paragraphs 57 to 62, wherein each R^{e} is independently selected from fluoro, chloro, CN, CF₃, OCF₃, and methyl.

64. A compound for use/a method according to any one of paragraphs 57 to 60, wherein R^{c1} and R^{d1} are independently selected from C₁₋₆ alkyl.

65. A compound for use/a method according to any one of paragraphs 1 to 36, wherein the compound of formula I is selected from:
2-chloro-N-(2-(trifluoromethyl)benzyl)pyrido[2,3-d]pyrimidin-4-amine
N²-isopropyl-N⁴-(2-(trifluoromethyl)benzyl)pyrido[2,3-d]pyrimidine-2,4-diamine
N⁴-(4-cyanobenzyl)-N²-isopropylpyrido[2,3-d]pyrimidine-2,4-diamine
N²-isopropyl-N⁴-(2-(methylsulfonyl)benzyl)pyrido[2,3-d]pyrimidine-2,4-diamine
N²-isopropyl-N⁴-(4-(methylsulfonyl)benzyl)pyrido[2,3-d]pyrimidine-2,4-diamine
N²-isopropyl-N⁴-(4-(trifluoromethyl)benzyl)pyrido[2,3-d]pyrimidine-2,4-diamine
N²-isopropyl-N⁴-((6-(trifluoromethyl)pyridin-3-yl)methyl)pyrido[2,3-d]pyrimidine-2,4-diamine
N²-(azetidin-3-yl)-N⁴-(2-(trifluoromethyl)benzyl)pyrido[2,3-d]pyrimidine-2,4-diamine
N²-(1-methylazetidin-3-yl)-N⁴-(2-(trifluoromethyl)benzyl)pyrido[2,3-d]pyrimidine-2,4-diamine
N²-methyl-N⁴-(2-(trifluoromethyl)benzyl)pyrido[2,3-d]pyrimidine-2,4-diamine
N²-(oxetan-3-yl)-N⁴-(2-(trifluoromethyl)benzyl)pyrido[2,3-d]pyrimidine-2,4-diamine
2-((methylamino)methyl)-N-(2-(trifluoromethyl)benzyl)pyrido[2,3-d]pyrimidin-4-amine
4-((2-(trifluoromethyl)benzyl)amino)pyrido[2,3-d]pyrimidine-2-carboxamide
N²-(azetidin-3-yl)-N⁴-((2-(trifluoromethyl)pyridin-3-yl)methyl)pyrido[2,3-d]pyrimidine-2,4-diamine
N²-(1-methylazetidin-3-yl)-N⁴-((2-(trifluoromethyl)pyridin-3-yl)methyl)pyrido[2,3-d]pyrimidine-2,4-diamine
N²-methyl-N⁴-((2-(trifluoromethyl)pyridin-3-yl)methyl)pyrido[2,3-d]pyrimidine-2,4-diamine
N²-isopropyl-N⁴-((2-(trifluoromethyl)pyridin-3-yl)methyl)pyrido[2,3-d]pyrimidine-2,4-diamine
N²-cyclopropyl-N⁴-(2-(trifluoromethyl)benzyl)pyrido[2,3-d]pyrimidine-2,4-diamine
4-((2-(trifluoromethyl)benzyl)amino)pyrido[2,3-d]pyrimidin-2-ol
N²-(oxetan-3-yl)-N⁴-((2-(trifluoromethyl)pyridin-3-yl)methyl)pyrido[2,3-d]pyrimidine-2,4-diamine
N²-cyclopropyl-N⁴-((2-(trifluoromethyl)pyridin-3-yl)methyl)pyrido[2,3-d]pyrimidine-2,4-diamine
N²-isopropyl-N⁴-methyl-N⁴-((2-(trifluoromethyl)pyridin-3-yl)methyl)pyrido[2,3-d]pyrimidine-2,4-diamine
N²-isopropyl-N², N⁴-dimethyl-N⁴-((2-(trifluoromethyl)pyridin-3-yl)methyl)pyrido[2,3-d]pyrimidine-2,4-diamine
N²-isopropyl-N²-methyl-N⁴-((2-(trifluoromethyl)pyridin-3-yl)methyl)pyrido[2,3-d]pyrimidine-2,4-diamine
2-(azetidin-1-yl)-N-((2-(trifluoromethyl)pyridin-3-yl)methyl)pyrido[2,3-d]pyrimidin-4-amine
2-(isopropylthio)-N-((2-(trifluoromethyl)pyridin-3-yl)methyl)pyrido[2,3-d]pyrimidin-4-amine
N⁴-(2-fluoro-6-(trifluoromethyl)benzyl)-N²-isopropylpyrido[2,3-d]pyrimidine-2,4-diamine
N²-(tert-butyl)-N⁴-((2-(trifluoromethyl)pyridin-3-yl)methyl)pyrido[2,3-d]pyrimidine-2,4-diamine
N⁴-(2-fluoro-6-(trifluoromethyl)benzyl)-N², N²-dimethylpyrido[2,3-d]pyrimidine-2,4-diamine
N-((2-(trifluoromethyl)pyridin-3-yl)methyl)pyrido[2,3-d]pyrimidin-4-amine
3-methyl-1-(4-(((2-(trifluoromethyl)pyridin-3-yl)methyl)amino)pyrido[2,3-d]pyrimidin-2-yl)azetidin-3-ol
2-(2-methylazetidin-1-yl)-N-((2-(trifluoromethyl)pyridin-3-yl)methyl)pyrido[2,3-d]pyrimidin-4-amine
2-(2,2-dimethylazetidin-1-yl)-N-((2-(trifluoromethyl)pyridin-3-yl)methyl)pyrido[2,3-d]pyrimidin-4-amine
2-(pyrrolidin-1-yl)-N-((2-(trifluoromethyl)pyridin-3-yl)methyl)pyrido[2,3-d]pyrimidin-4-amine
N²-cyclopropyl-N²-methyl-N⁴-((2-(trifluoromethyl)pyridin-3-yl)methyl)pyrido[2,3-d]pyrimidine-2,4-diamine
2-methyl-N-((2-(trifluoromethyl)pyridin-3-yl)methyl)pyrido[2,3-d]pyrimidin-4-amine
2-(isopropylsulfinyl)-N-((2-(trifluoromethyl)pyridin-3-yl)methyl)pyrido[2,3-d]pyrimidin-4-amine
2-(isopropylsulfonyl)-N-((2-(trifluoromethyl)pyridin-3-yl)methyl)pyrido[2,3-d]pyrimidin-4-amine
4-(((2-(trifluoromethyl)pyridin-3-yl)methyl)amino)pyrido[2,3-d]pyrimidin-2-ol
N-isopropyl-4-(2-(2-(trifluoromethyl)phenyl)azetidin-1-yl)pyrido[2,3-d]pyrimidin-2-amine
N-isopropyl-N-methyl-4-(2-(2-(trifluoromethyl)phenyl)azetidin-1-yl)pyrido[2,3-d]pyrimidin-2-amine
N-isopropyl-4-(2-(2-(trifluoromethyl)phenyl)piperidin-1-yl)pyrido[2,3-d]pyrimidin-2-amine
N-isopropyl-N-methyl-4-(2-(2-(trifluoromethyl)phenyl)piperidin-1-yl)pyrido[2,3-d]pyrimidin-2-amine
N-isopropyl-4-(3-(2-(trifluoromethyl)phenyl)morpholino)pyrido[2,3-d]pyrimidin-2-amine
N-isopropyl-N-methyl-4-(3-(2-(trifluoromethyl)phenyl)morpholino)pyrido[2,3-d]pyrimidin-2-amine
N-isopropyl-4-(2-(2-(trifluoromethyl)phenyl)pyrazolidin-1-yl)pyrido[2,3-d]pyrimidin-2-amine
N-isopropyl-N-methyl-4-(2-(2-(trifluoromethyl)phenyl)pyrazolidin-1-yl)pyrido[2,3-d]pyrimidin-2-amine
2-((difluoromethyl)thio)-N-((2-(trifluoromethyl)pyridin-3-yl)methyl)pyrido[2,3-d]pyrimidin-4-amine
2-morpholino-N-((2-(trifluoromethyl)pyridin-3-yl)methyl)pyrido[2,3-d]pyrimidin-4-amine
2-(3-methylmorpholino)-N-((2-(trifluoromethyl)pyridin-3-yl)methyl)pyrido[2,3-d]pyrimidin-4-amine
2-(3,3-difluoropyrrolidin-1-yl)-N-((2-(trifluoromethyl)pyridin-3-yl)methyl)pyrido[2,3-d]pyrimidin-4-amine
2-(4-fluoropiperidin-1-yl)-N-((2-(trifluoromethyl)pyridin-3-yl)methyl)pyrido[2,3-d]pyrimidin-4-amine
2-(4,4-difluoropiperidin-1-yl)-N-(2-(trifluoromethyl)benzyl)pyrido[2,3-d]pyrimidin-4-amine
2-(4,4-difluoropiperidin-1-yl)-N-((2-(trifluoromethyl)pyridin-3-yl)methyl)pyrido[2,3-d]pyrimidin-4-amine
2-(4-chloropiperidin-1-yl)-N-(2-(trifluoromethyl)benzyl)pyrido[2,3-d]pyrimidin-4-amine
2-(4-chloropiperidin-1-yl)-N-((2-(trifluoromethyl)pyridin-3-yl)methyl)pyrido[2,3-d]pyrimidin-4-amine
2-(3-fluoroazetidin-1-yl)-N-((2-(trifluoromethyl)pyridin-3-yl)methyl)pyrido[2,3-d]pyrimidin-4-amine
2-(3,3-difluoroazetidin-1-yl)-N-((2-(trifluoromethyl)pyridin-3-yl)methyl)pyrido[2,3-d]pyrimidin-4-amine
2-(3-(trifluoromethyl)azetidin-1-yl)-N-((2-(trifluoromethyl)pyridin-3-yl)methyl)pyrido[2,3-d]pyrimidin-4-amine
2-(4-(trifluoromethyl)-1H-pyrazol-1-yl)-N-((2-(trifluoromethyl)pyridin-3-yl)methyl)pyrido[2,3-d]pyrimidin-4-amine
1-(4-fluorophenyl)-3-(4-(((2-(trifluoromethyl)pyridin-3-yl)methyl)amino)pyrido[2,3-d]pyrimidin-2-yl)urea
1-(3-fluorophenyl)-3-(4-(((2-(trifluoromethyl)pyridin-3-yl)methyl)amino)pyrido[2,3-d]pyrimidin-2-yl)urea
1-(2-fluorophenyl)-3-(4-(((2-(trifluoromethyl)pyridin-3-yl)methyl)amino)pyrido[2,3-d]pyrimidin-2-yl)urea
1-ethyl-3-(4-(((2-(trifluoromethyl)pyridin-3-yl)methyl)amino)pyrido[2,3-d]pyrimidin-2-yl)urea
(S)-2-(3-methylmorpholino)-N-(2-(trifluoromethyl)benzyl)pyrido[2,3-d]pyrimidin-4-amine
(R)-2-(3-methylmorpholino)-N-(2-(trifluoromethyl)benzyl)pyrido[2,3-d]pyrimidin-4-amine
(S)-2-(3-methylmorpholino)-N-((2-(trifluoromethyl)pyridin-3-yl)methyl)pyrido[2,3-d]pyrimidin-4-amine
(R)-2-(3-methylmorpholino)-N-((2-(trifluoromethyl)pyridin-3-yl)methyl)pyrido[2,3-d]pyrimidin-4-amine
2-(4-chloropiperidin-1-yl)-4-(2-(2-(trifluoromethyl)phenyl)azetidin-1-yl)pyrido[2,3-d]pyrimidine
2-(4,4-difluoropiperidin-1-yl)-4-(2-(2-(trifluoromethyl)phenyl)azetidin-1-yl)pyrido[2,3-d]pyrimidine
3-methyl-4-(4-(2-(2-(trifluoromethyl)phenyl)azetidin-1-yl)pyrido[2,3-d]pyrimidin-2-yl)morpholine
2-(3,3-difluoropyrrolidin-1-yl)-4-(2-(2-(trifluoromethyl)phenyl)azetidin-1-yl)pyrido[2,3-d]pyrimidine
2-(3-(trifluoromethyl)azetidin-1-yl)-4-(2-(2-(trifluoromethyl)phenyl)azetidin-1-yl)pyrido[2,3-d]pyrimidine
2-(4-chloropiperidin-1-yl)-4-(2-(2-(trifluoromethyl)pyridin-3-yl)azetidin-1-yl)pyrido[2,3-d]pyrimidine
2-(4,4-difluoropiperidin-1-yl)-4-(2-(2-(trifluoromethyl)pyridin-3-yl)azetidin-1-yl)pyrido[2,3-d]pyrimidine
3-methyl-4-(4-(2-(2-(trifluoromethyl)pyridin-3-yl)azetidin-1-yl)pyrido[2,3-d]pyrimidin-2-yl)morpholine
2-(3,3-difluoropyrrolidin-1-yl)-4-(2-(2-(trifluoromethyl)pyridin-3-yl)azetidin-1-yl)pyrido[2,3-d]pyrimidine
2-(3-(trifluoromethyl)azetidin-1-yl)-4-(2-(2-(trifluoromethyl)pyridin-3-yl)azetidin-1-yl)pyrido[2,3-d]pyrimidine
2-((4-chloropiperidin-1-yl)methyl)-N-((2-(trifluoromethyl)pyridin-3-yl)methyl)pyrido[2,3-d]pyrimidin-4-amine
2-((4,4-difluoropiperidin-1-yl)methyl)-N-((2-(trifluoromethyl)pyridin-3-yl)methyl)pyrido[2,3-d]pyrimidin-4-amine
2-((3-methylmorpholino)methyl)-N-((2-(trifluoromethyl)pyridin-3-yl)methyl)pyrido[2,3-d]pyrimidin-4-amine
2-((3,3-difluoropyrrolidin-1-yl)methyl)-N-((2-(trifluoromethyl)pyridin-3-yl)methyl)pyrido[2,3-d]pyrimidin-4-amine
2-((3-(trifluoromethyl)azetidin-1-yl)methyl)-N-((2-(trifluoromethyl)pyridin-3-yl)methyl)pyrido[2,3-d]pyrimidin-4-amine
2-((4-chloropiperidin-1-yl)methyl)-N-((2-(trifluoromethyl)pyridin-3-yl)methyl)pyrido[2,3-d]pyrimidin-4-amine
2-((4,4-difluoropiperidin-1-yl)methyl)-N-((2-(trifluoromethyl)pyridin-3-yl)methyl)pyrido[2,3-d]pyrimidin-4-amine
2-((3-methylmorpholino)methyl)-N-((2-(trifluoromethyl)pyridin-3-yl)methyl)pyrido[2,3-d]pyrimidin-4-amine
2-((3,3-difluoropyrrolidin-1-yl)methyl)-N-((2-(trifluoromethyl)pyridin-3-yl)methyl)pyrido[2,3-d]pyrimidin-4-amine
2-((3-(trifluoromethyl)azetidin-1-yl)methyl)-N-((2-(trifluoromethyl)pyridin-3-yl)methyl)pyrido[2,3-d]pyrimidin-4-amine
2-(4-(methylsulfonyl)piperidin-1-yl)-N-((2-(trifluoromethyl)pyridin-3-yl)methyl)pyrido[2,3-d]pyrimidin-4-amine
2-(4,4-dimethylpiperidin-1-yl)-N-((2-(trifluoromethyl)pyridin-3-yl)methyl)pyrido[2,3-d]pyrimidin-4-amine
1-(4-(((2-(trifluoromethyl)pyridin-3-yl)methyl)amino)pyrido[2,3-d]pyrimidin-2-yl)piperidine-4-carbonitrile
2-(4-(trifluoromethyl)piperidin-1-yl)-N-((2-(trifluoromethyl)pyridin-3-yl)methyl)pyrido[2,3-d]pyrimidin-4-amine
N-((2-(trifluoromethyl)pyridin-3-yl)methyl)-2-(3-(trifluoromethyl) pyrrolidin-1-yl)pyrido[2,3-d]pyrimidin-4-amine
1-(4-(((2-(trifluoromethyl)pyridin-3-yl)methyl)amino)pyrido[2,3-d]pyrimidin-2-yl)azetidine-3-carbonitrile
1-(4-(((2-(trifluoromethyl)pyridin-3-yl)methyl)amino)pyrido[2,3-d]pyrimidin-2-yl)pyrrolidine-2-carbonitrile
2-(2,2-dimethylmorpholino)-N-((2-(trifluoromethyl)pyridin-3-yl)methyl)pyrido[2,3-d]pyrimidin-4-amine
2-(3,3-dimethylmorpholino)-N-((2-(trifluoromethyl)pyridin-3-yl)methyl)pyrido[2,3-d]pyrimidin-4-amine
2-(2-methylmorpholino)-N-((2-(trifluoromethyl)pyridin-3-yl)methyl)pyrido[2,3-d]pyrimidin-4-amine
2-(3-fluoropyrrolidin-1-yl)-N-((2-(trifluoromethyl)pyridin-3-yl)methyl)pyrido[2,3-d]pyrimidin-4-amine
2-(4-(methylsulfonyl)piperidin-1-yl)-N-(2-(trifluoromethyl)benzyl)pyrido[2,3-d]pyrimidin-4-amine
2-(4,4-dimethylpiperidin-1-yl)-N-(2-(trifluoromethyl)benzyl)pyrido[2,3-d]pyrimidin-4-amine
1-(4-((2-(trifluoromethyl)benzyl)amino)pyrido[2,3-d]pyrimidin-2-yl)azetidine-3-carbonitrile
2-(3,3-dimethylmorpholino)-N-(2-(trifluoromethyl)benzyl)pyrido[2,3-d]pyrimidin-4-amine
2-(2,2-dimethylmorpholino)-N-(2-(trifluoromethyl)benzyl)pyrido[2,3-d]pyrimidin-4-amine
2-(2-methylmorpholino)-N-(2-(trifluoromethyl)benzyl)pyrido[2,3-d]pyrimidin-4-amine
2-(3-(trifluoromethyl)piperidin-1-yl)-N-((2-(trifluoromethyl)pyridin-3-yl)methyl)pyrido[2,3-d]pyrimidin-4-amine
4-methyl-1-(4-(((2-(trifluoromethyl)pyridin-3-yl)methyl)amino)pyrido[2,3-d]pyrimidin-2-yl)piperidine-4-carbonitrile
1-(4-(((2-(trifluoromethyl)pyridin-3-yl)methyl)amino)pyrido[2,3-d]pyrimidin-2-yl)piperidine-3-carbonitrile
2-(2,2-difluoro-7-azaspiro[3.5]nonan-7-yl)-N-((2-(trifluoromethyl)pyridin-3-yl)methyl)pyrido[2,3-d]pyrimidin-4-amine
2-(3-cyclopropylmorpholino)-N-((2-(trifluoromethyl)pyridin-3-yl)methyl)pyrido[2,3-d]pyrimidin-4-amine
2-((6S)-2,6-dimethylmorpholino)-N-((2-(trifluoromethyl)pyridin-3-yl)methyl)pyrido[2,3-d]pyrimidin-4-amine
2-(2-oxa-5-azabicyclo[2.2.1]heptan-5-yl)-N-((2-(trifluoromethyl)pyridin-3-yl)methyl)pyrido[2,3-d]pyrimidin-4-amine
2-(4-oxa-7-azaspiro[2.5]octan-7-yl)-N-((2-(trifluoromethyl)pyridin-3-yl)methyl)pyrido[2,3-d]pyrimidin-4-amine
4-(4-(((2-(trifluoromethyl)pyridin-3-yl)methyl)amino)pyrido[2,3-d]pyrimidin-2-yl)morpholine-2-carbonitrile
2-(3-(fluoromethyl)piperidin-1-yl)-N-((2-(trifluoromethyl)pyridin-3-yl)methyl)pyrido[2,3-d]pyrimidin-4-amine
2-(2-(trifluoromethyl)piperidin-1-yl)-N-((2-(trifluoromethyl)pyridin-3-yl)methyl)pyrido[2,3-d]pyrimidin-4-amine
2-(2,2-difluoro-7-azaspiro[3.5]nonan-7-yl)-N-(2-(trifluoromethyl)benzyl)pyrido[2,3-d]pyrimidin-4-amine
4-methyl-1-(4-((2-(trifluoromethyl)benzyl)amino)pyrido[2,3-d]pyrimidin-2-yl)piperidine-4-carbonitrile
1-(4-((2-(trifluoromethyl)benzyl)amino)pyrido[2,3-d]pyrimidin-2-yl)piperidine-3-carbonitrile
2-(3-(methylsulfonyl)pyrrolidin-1-yl)-N-((2-(trifluoromethyl)pyridin-3-yl)methyl)pyrido[2,3-d]pyrimidin-4-amine
N-((2-(trifluoromethyl)pyridin-3-yl)methyl)-2-(2-(trifluoromethyl) pyrrolidin-1-yl)pyrido[2,3-d]pyrimidin-4-amine
2-(3-chloropyrrolidin-1-yl)-N-((2-(trifluoromethyl)pyridin-3-yl)methyl)pyrido[2,3-d]pyrimidin-4-amine
N-(2-(trifluoromethyl)benzyl)-2-(3-(trifluoromethyl) pyrrolidin-1-yl)pyrido[2,3-d]pyrimidin-4-amine
2-(2-oxa-5-azabicyclo[2.2.1]heptan-5-yl)-N-(2-(trifluoromethyl)benzyl)pyrido[2,3-d]pyrimidin-4-amine
2-((6S)-2,6-dimethylmorpholino)-N-(2-(trifluoromethyl)benzyl)pyrido[2,3-d]pyrimidin-4-amine
2-(3-isopropylmorpholino)-N-((2-(trifluoromethyl)pyridin-3-yl)methyl)pyrido[2,3-d]pyrimidin-4-amine
2-((2R,3R)-2,3-dimethylmorpholino)-N-((2-(trifluoromethyl)pyridin-3-yl)methyl)pyrido[2,3-d]pyrimidin-4-amine
2-((2S,5R)-2,5-dimethylmorpholino)-N-((2-(trifluoromethyl)pyridin-3-yl)methyl)pyrido[2,3-d]pyrimidin-4-amine
2-(6-oxa-9-azaspiro[4.5]decan-9-yl)-N-((2-(trifluoromethyl)pyridin-3-yl)methyl)pyrido[2,3-d]pyrimidin-4-amine
2-(4-(trifluoromethoxy)piperidin-1-yl)-N-((2-(trifluoromethyl)pyridin-3-yl)methyl)pyrido[2,3-d]pyrimidin-4-amine
2-(3-(difluoromethyl)azetidin-1-yl)-N-((2-(trifluoromethyl)pyridin-3-yl)methyl)pyrido[2,3-d]pyrimidin-4-amine
2-((3R)-3,5-dimethylmorpholino)-N-((2-(trifluoromethyl)pyridin-3-yl)methyl)pyrido[2,3-d]pyrimidin-4-amine
2-(3-(difluoromethyl)azetidin-1-yl)-N-(2-(trifluoromethyl)benzyl)pyrido[2,3-d]pyrimidin-4-amine
2-((3R)-3,5-dimethylmorpholino)-N-(2-(trifluoromethyl)benzyl)pyrido[2,3-d]pyrimidin-4-amine
2-(2-cyclopropylmorpholino)-N-((2-(trifluoromethyl)pyridin-3-yl)methyl)pyrido[2,3-d]pyrimidin-4-amine
2-((2S,5S)-2,5-dimethylmorpholino)-N-((2-(trifluoromethyl)pyridin-3-yl)methyl)pyrido[2,3-d]pyrimidin-4-amine
(R)-2-(3-(difluoromethoxy)pyrrolidin-1-yl)-N-((2-(trifluoromethyl)pyridin-3-yl)methyl)pyrido[2,3-d]pyrimidin-4-amine
(S)-2-(3-(difluoromethoxy)pyrrolidin-1-yl)-N-((2-(trifluoromethyl)pyridin-3-yl)methyl)pyrido[2,3-d]pyrimidin-4-amine
2-(2-(difluoromethyl)morpholino)-N-((2-(trifluoromethyl)pyridin-3-yl)methyl)pyrido[2,3-d]pyrimidin-4-amine
4-(4-((2-(trifluoromethyl)benzyl)amino)pyrido[2,3-d]pyrimidin-2-yl)morpholine-2-carbonitrile
2-(2-(difluoromethyl)morpholino)-N-(2-(trifluoromethyl)benzyl)pyrido[2,3-d]pyrimidin-4-amine
2-(8-oxa-5-azaspiro[3.5]nonan-5-yl)-N-(2-(trifluoromethyl)benzyl)pyrido[2,3-d]pyrimidin-4-amine
2-(9-oxa-6-azaspiro[4.5]decan-6-yl)-N-(2-(trifluoromethyl)benzyl)pyrido[2,3-d]pyrimidin-4-amine
2-((2R,3S)-2,3-dimethylmorpholino)-N-((2-(trifluoromethyl)pyridin-3-yl)methyl)pyrido[2,3-d]pyrimidin-4-amine
(3R)-3-methyl-4-(4-(2-(2-(trifluoromethyl)pyridin-3-yl)azetidin-1-yl)pyrido[2,3-d]pyrimidin-2-yl)morpholine
(3S)-3-methyl-4-(4-(2-(2-(trifluoromethyl)pyridin-3-yl)azetidin-1-yl)pyrido[2,3-d]pyrimidin-2-yl)morpholine
(3R)-3-methyl-4-(4-(2-(2-(trifluoromethyl)phenyl)azetidin-1-yl)pyrido[2,3-d]pyrimidin-2-yl)morpholine
(3S)-3-methyl-4-(4-(2-(2-(trifluoromethyl)phenyl)azetidin-1-yl)pyrido[2,3-d]pyrimidin-2-yl)morpholine
2-(3-(difluoromethoxy)piperidin-1-yl)-N-((2-(trifluoromethyl)pyridin-3-yl)methyl)pyrido[2,3-d]pyrimidin-4-amine
2-(6-oxa-3-azabicyclo[3.1.1]heptan-3-yl)-N-((2-(trifluoromethyl)pyridin-3-yl)methyl)pyrido[2,3-d]pyrimidin-4-amine
2-(2-oxa-5-azabicyclo[4.1.0]heptan-5-yl)-N-((2-(trifluoromethyl)pyridin-3-yl)methyl)pyrido[2,3-d]pyrimidin-4-amine
2-(2,2,6,6-tetrafluoromorpholino)-N-((2-(trifluoromethyl)pyridin-3-yl)methyl)pyrido[2,3-d]pyrimidin-4-amine
2-(4-azaspiro[2.5]octan-4-yl)-N-((2-(trifluoromethyl)pyridin-3-yl)methyl)pyrido[2,3-d]pyrimidin-4-amine
2-(3-(trifluoromethoxy)pyrrolidin-1-yl)-N-((2-(trifluoromethyl)pyridin-3-yl)methyl)pyrido[2,3-d]pyrimidin-4-amine
2-(5-azaspiro[3.4]octan-5-yl)-N-((2-(trifluoromethyl)pyridin-3-yl)methyl)pyrido[2,3-d]pyrimidin-4-amine
2-(2-((trifluoromethoxy)methyl)pyrrolidin-1-yl)-N-((2-(trifluoromethyl)pyridin-3-yl)methyl)pyrido[2,3-d]pyrimidin-4-amine
2-(6-oxa-3-azabicyclo[3.1.1]heptan-3-yl)-N-(2-(trifluoromethyl)benzyl)pyrido[2,3-d]pyrimidin-4-amine
2-(2-oxa-5-azabicyclo[4.1.0]heptan-5-yl)-N-(2-(trifluoromethyl)benzyl)pyrido[2,3-d]pyrimidin-4-amine
2-((3R)-3,5-dimethylmorpholino)-N-(2-(trifluoromethyl)benzyl)pyrido[2,3-d]pyrimidin-4-amine
6-fluoro-2-(3-methylmorpholino)-N-((2-(trifluoromethyl)pyridin-3-yl)methyl)pyrido[2,3-d]pyrimidin-4-amine
6-methoxy-2-(3-methylmorpholino)-N-((2-(trifluoromethyl)pyridin-3-yl)methyl)pyrido[2,3-d]pyrimidin-4-amine
7-methoxy-2-(3-methylmorpholino)-N-((2-(trifluoromethyl)pyridin-3-yl)methyl)pyrido[2,3-d]pyrimidin-4-amine
5-methoxy-2-(3-methylmorpholino)-N-((2-(trifluoromethyl)pyridin-3-yl)methyl)pyrido[2,3-d]pyrimidin-4-amine
6,7-dimethoxy-2-(3-methylmorpholino)-N-((2-(trifluoromethyl)pyridin-3-yl)methyl)pyrido[2,3-d]pyrimidin-4-amine, and
2-(3-methylmorpholino)-7-(2-morpholinoethoxy)-N-((2-(trifluoromethyl)pyridin-3-yl)methyl)pyrido[2,3-d]pyrimidin-4-amine;
   or a pharmaceutically acceptable salt or solvate thereof.

66. A compound for use/a method according to paragraph 65, wherein the compound of formula I is selected from:
N²-isopropyl-N²,N⁴-dimethyl-N⁴-((2-(trifluoromethyl)pyridin-3-yl)methyl)pyrido[2,3-d]pyrimidine-2,4-diamine;
N²-isopropyl-N²-methyl-N⁴-((2-(trifluoromethyl)pyridin-3-yl)methyl)pyrido[2,3-d]pyrimidine-2,4-diamine;
N²-isopropyl-N²-methyl-N⁴-((2-(trifluoromethyl)pyridin-3-yl)methyl)pyrido[2,3-d]pyrimidine-2,4-diamine methanesulfonate;
2-(azetidin-1-yl)-N-((2-(trifluoromethyl)pyridin-3-yl)methyl)pyrido[2,3-d]pyrimidin-4-amine;
N⁴-(2-fluoro-6-(trifluoromethyl)benzyl)-N²,N²-dimethylpyrido[2,3-d]pyrimidine-2,4-diamine;
3-methyl-1-(4-(((2-(trifluoromethyl)pyridin-3-yl)methyl)amino)pyrido[2,3-d]pyrimidin-2-yl)azetidin-3-ol;
2-(2-methylazetidin-1-yl)-N-((2-(trifluoromethyl)pyridin-3-yl)methyl)pyrido[2,3-d]pyrimidin-4-amine;
2-(2,2-dimethylazetidin-1-yl)-N-((2-(trifluoromethyl)pyridin-3-yl)methyl)pyrido[2,3-d]pyrimidin-4-amine;
2-(pyrrolidin-1-yl)-N-((2-(trifluoromethyl)pyridin-3-yl)methyl)pyrido[2,3-d]pyrimidin-4-amine;
N²-cyclopropyl-N²-methyl-N⁴-((2-(trifluoromethyl)pyridin-3-yl)methyl)pyrido[2,3-d]pyrimidine-2,4-diamine;
N-isopropyl-N-methyl-4-(2-(2-(trifluoromethyl)phenyl)azetidin-1-yl)pyrido[2,3-d]pyrimidin-2-amine;
N-isopropyl-N-methyl-4-(2-(2-(trifluoromethyl)phenyl)piperidin-1-yl)pyrido[2,3-d]pyrimidin-2-amine;
N-isopropyl-N-methyl-4-(3-(2-(trifluoromethyl)phenyl)morpholino)pyrido[2,3-d]pyrimidin-2-amine;
N-isopropyl-N-methyl-4-(2-(2-(trifluoromethyl)phenyl)pyrazolidin-1-yl)pyrido[2,3-d]pyrimidin-2-amine;
2-morpholino-N-((2-(trifluoromethyl)pyridin-3-yl)methyl)pyrido[2,3-d]pyrimidin-4-amine;
2-(3-methylmorpholino)-N-((2-(trifluoromethyl)pyridin-3-yl)methyl)pyrido[2,3-d]pyrimidin-4-amine;
2-(3-methylmorpholino)-N-((2-(trifluoromethyl)pyridin-3-yl)methyl)pyrido[2,3-d]pyrimidin-4-amine methanesulfonate;
2-(3,3-difluoropyrrolidin-1-yl)-N-((2-(trifluoromethyl)pyridin-3-yl)methyl)pyrido[2,3-d]pyrimidin-4-amine;
2-(3,3-difluoropyrrolidin-1-yl)-N-((2-(trifluoromethyl)pyridin-3-yl)methyl)pyrido[2,3-d]pyrimidin-4-amine methanesulfonate;
2-(4-fluoropiperidin-1-yl)-N-((2-(trifluoromethyl)pyridin-3-yl)methyl)pyrido[2,3-d]pyrimidin-4-amine;
2-(4,4-difluoropiperidin-1-yl)-N-(2-(trifluoromethyl)benzyl)pyrido[2,3-d]pyrimidin-4-amine;
2-(4,4-difluoropiperidin-1-yl)-N-((2-(trifluoromethyl)pyridin-3-yl)methyl)pyrido[2,3-d]pyrimidin-4-amine;
2-(4-chloropiperidin-1-yl)-N-(2-(trifluoromethyl)benzyl)pyrido[2,3-d]pyrimidin-4-amine;
2-(4-chloropiperidin-1-yl)-N-((2-(trifluoromethyl)pyridin-3-yl)methyl)pyrido[2,3-d]pyrimidin-4-amine;
2-(3-fluoroazetidin-1-yl)-N-((2-(trifluoromethyl)pyridin-3-yl)methyl)pyrido[2,3-d]pyrimidin-4-amine;
2-(3,3-difluoroazetidin-1-yl)-N-((2-(trifluoromethyl)pyridin-3-yl)methyl)pyrido[2,3-d]pyrimidin-4-amine;
2-(3-(trifluoromethyl)azetidin-1-yl)-N-((2-(trifluoromethyl)pyridin-3-yl)methyl)pyrido[2,3-d]pyrimidin-4-amine;
(S)-2-(3-methylmorpholino)-N-(2-(trifluoromethyl)benzyl)pyrido[2,3-d]pyrimidin-4-amine;
(S)-2-(3-methylmorpholino)-N-(2-(trifluoromethyl)benzyl)pyrido[2,3-d]pyrimidin-4-amine methanesulfonate;
(R)-2-(3-methylmorpholino)-N-(2-(trifluoromethyl)benzyl)pyrido[2,3-d]pyrimidin-4-amine;
(S)-2-(3-methylmorpholino)-N-((2-(trifluoromethyl)pyridin-3-yl)methyl)pyrido[2,3-d]pyrimidin-4-amine;
(R)-2-(3-methylmorpholino)-N-((2-(trifluoromethyl)pyridin-3-yl)methyl)pyrido[2,3-d]pyrimidin-4-amine;
2-(4-chloropiperidin-1-yl)-4-(2-(2-(trifluoromethyl)phenyl)azetidin-1-yl)pyrido[2,3-d]pyrimidine;
2-(4,4-difluoropiperidin-1-yl)-4-(2-(2-(trifluoromethyl)phenyl)azetidin-1-yl)pyrido[2,3-d]pyrimidine;
3-methyl-4-(4-(2-(2-(trifluoromethyl)phenyl)azetidin-1-yl)pyrido[2,3-d]pyrimidin-2-yl)morpholine;
2-(3,3-difluoropyrrolidin-1-yl)-4-(2-(2-(trifluoromethyl)phenyl)azetidin-1-yl)pyrido[2,3-d]pyrimidine;
2-(3-(trifluoromethyl)azetidin-1-yl)-4-(2-(2-(trifluoromethyl)phenyl)azetidin-1-yl)pyrido[2,3-d]pyrimidine;
2-(4-chloropiperidin-1-yl)-4-(2-(2-(trifluoromethyl)pyridin-3-yl)azetidin-1-yl)pyrido[2,3-d]pyrimidine;
2-(4,4-difluoropiperidin-1-yl)-4-(2-(2-(trifluoromethyl)pyridin-3-yl)azetidin-1-yl)pyrido[2,3-d]pyrimidine;
3-methyl-4-(4-(2-(2-(trifluoromethyl)pyridin-3-yl)azetidin-1-yl)pyrido[2,3-d]pyrimidin-2-yl)morpholine;
2-(3,3-difluoropyrrolidin-1-yl)-4-(2-(2-(trifluoromethyl)pyridin-3-yl)azetidin-1-yl)pyrido[2,3-d]pyrimidine;
2-(3-(trifluoromethyl)azetidin-1-yl)-4-(2-(2-(trifluoromethyl)pyridin-3-yl)azetidin-1-yl)pyrido[2,3-d]pyrimidine;
2-((4-chloropiperidin-1-yl)methyl)-N-((2-(trifluoromethyl)pyridin-3-yl)methyl)pyrido[2,3-d]pyrimidin-4-amine;
2-((4,4-difluoropiperidin-1-yl)methyl)-N-((2-(trifluoromethyl)pyridin-3-yl)methyl)pyrido[2,3-d]pyrimidin-4-amine;
2-((3-methylmorpholino)methyl)-N-((2-(trifluoromethyl)pyridin-3-yl)methyl)pyrido[2,3-d]pyrimidin-4-amine;
2-((3,3-difluoropyrrolidin-1-yl)methyl)-N-((2-(trifluoromethyl)pyridin-3-yl)methyl)pyrido[2,3-d]pyrimidin-4-amine;
2-((3-(trifluoromethyl)azetidin-1-yl)methyl)-N-((2-(trifluoromethyl)pyridin-3-yl)methyl)pyrido[2,3-d]pyrimidin-4-amine;
2-((4-chloropiperidin-1-yl)methyl)-N-((2-(trifluoromethyl)pyridin-3-yl)methyl)pyrido[2,3-d]pyrimidin-4-amine;
2-((4,4-difluoropiperidin-1-yl)methyl)-N-((2-(trifluoromethyl)pyridin-3-yl)methyl)pyrido[2,3-d]pyrimidin-4-amine;
2-((3-methylmorpholino)methyl)-N-((2-(trifluoromethyl)pyridin-3-yl)methyl)pyrido[2,3-d]pyrimidin-4-amine;
2-((3,3-difluoropyrrolidin-1-yl)methyl)-N-((2-(trifluoromethyl)pyridin-3-yl)methyl)pyrido[2,3-d]pyrimidin-4-amine;
2-((3-(trifluoromethyl)azetidin-1-yl)methyl)-N-((2-(trifluoromethyl)pyridin-3-yl)methyl)pyrido[2,3-d]pyrimidin-4-amine;
2-(4-(methylsulfonyl)piperidin-1-yl)-N-((2-(trifluoromethyl)pyridin-3-yl)methyl)pyrido[2,3-d]pyrimidin-4-amine;
2-(4,4-dimethylpiperidin-1-yl)-N-((2-(trifluoromethyl)pyridin-3-yl)methyl)pyrido[2,3-d]pyrimidin-4-amine;
1-(4-(((2-(trifluoromethyl)pyridin-3-yl)methyl)amino)pyrido[2,3-d]pyrimidin-2-yl)piperidine-4-carbonitrile;
2-(4-(trifluoromethyl)piperidin-1-yl)-N-((2-(trifluoromethyl)pyridin-3-yl)methyl)pyrido[2,3-d]pyrimidin-4-amine;
N-((2-(trifluoromethyl)pyridin-3-yl)methyl)-2-(3-(trifluoromethyl) pyrrolidin-1-yl)pyrido[2,3-d]pyrimidin-4-amine;
1-(4-(((2-(trifluoromethyl)pyridin-3-yl)methyl)amino)pyrido[2,3-d]pyrimidin-2-yl)azetidine-3-carbonitrile;
1-(4-(((2-(trifluoromethyl)pyridin-3-yl)methyl)amino)pyrido[2,3-d]pyrimidin-2-yl)pyrrolidine-2-carbonitrile;
2-(2,2-dimethylmorpholino)-N-((2-(trifluoromethyl)pyridin-3-yl)methyl)pyrido[2,3-d]pyrimidin-4-amine;
2-(3,3-dimethylmorpholino)-N-((2-(trifluoromethyl)pyridin-3-yl)methyl)pyrido[2,3-d]pyrimidin-4-amine;
2-(2-methylmorpholino)-N-((2-(trifluoromethyl)pyridin-3-yl)methyl)pyrido[2,3-d]pyrimidin-4-amine;
2-(3-fluoropyrrolidin-1-yl)-N-((2-(trifluoromethyl)pyridin-3-yl)methyl)pyrido[2,3-d]pyrimidin-4-amine;
2-(4-(methylsulfonyl)piperidin-1-yl)-N-(2-(trifluoromethyl)benzyl)pyrido[2,3-d]pyrimidin-4-amine;
2-(4,4-dimethylpiperidin-1-yl)-N-(2-(trifluoromethyl)benzyl)pyrido[2,3-d]pyrimidin-4-amine;
1-(4-((2-(trifluoromethyl)benzyl)amino)pyrido[2,3-d]pyrimidin-2-yl)azetidine-3-carbonitrile;
2-(3,3-dimethylmorpholino)-N-(2-(trifluoromethyl)benzyl)pyrido[2,3-d]pyrimidin-4-amine;
2-(2,2-dimethylmorpholino)-N-(2-(trifluoromethyl)benzyl)pyrido[2,3-d]pyrimidin-4-amine;
2-(2-methylmorpholino)-N-(2-(trifluoromethyl)benzyl)pyrido[2,3-d]pyrimidin-4-amine;
2-(3-(trifluoromethyl)piperidin-1-yl)-N-((2-(trifluoromethyl)pyridin-3-yl)methyl)pyrido[2,3-d]pyrimidin-4-amine;
4-methyl-1-(4-(((2-(trifluoromethyl)pyridin-3-yl)methyl)amino)pyrido[2,3-d]pyrimidin-2-yl)piperidine-4-carbonitrile;
1-(4-(((2-(trifluoromethyl)pyridin-3-yl)methyl)amino)pyrido[2,3-d]pyrimidin-2-yl)piperidine-3-carbonitrile;
2-(2,2-difluoro-7-azaspiro[3.5]nonan-7-yl)-N-((2-(trifluoromethyl)pyridin-3-yl)methyl)pyrido[2,3-d]pyrimidin-4-amine;
2-(2,2-difluoro-7-azaspiro[3.5]nonan-7-yl)-N-((2-(trifluoromethyl)pyridin-3-yl)methyl)pyrido[2,3-d]pyrimidin-4-amine methanesulfonate;
2-(3-cyclopropylmorpholino)-N-((2-(trifluoromethyl)pyridin-3-yl)methyl)pyrido[2,3-d]pyrimidin-4-amine;
2-((6S)-2,6-dimethylmorpholino)-N-((2-(trifluoromethyl)pyridin-3-yl)methyl)pyrido[2,3-d]pyrimidin-4-amine;
2-(2-oxa-5-azabicyclo[2.2.1]heptan-5-yl)-N-((2-(trifluoromethyl)pyridin-3-yl)methyl)pyrido[2,3-d]pyrimidin-4-amine;
2-(4-oxa-7-azaspiro[2.5]octan-7-yl)-N-((2-(trifluoromethyl)pyridin-3-yl)methyl)pyrido[2,3-d]pyrimidin-4-amine;
4-(4-(((2-(trifluoromethyl)pyridin-3-yl)methyl)amino)pyrido[2,3-d]pyrimidin-2-yl)morpholine-2-carbonitrile;
2-(3-(fluoromethyl)piperidin-1-yl)-N-((2-(trifluoromethyl)pyridin-3-yl)methyl)pyrido[2,3-d]pyrimidin-4-amine;
2-(2-(trifluoromethyl)piperidin-1-yl)-N-((2-(trifluoromethyl)pyridin-3-yl)methyl)pyrido[2,3-d]pyrimidin-4-amine;
2-(2,2-difluoro-7-azaspiro[3.5]nonan-7-yl)-N-(2-(trifluoromethyl)benzyl)pyrido[2,3-d]pyrimidin-4-amine;
4-methyl-1-(4-((2-(trifluoromethyl)benzyl)amino)pyrido[2,3-d]pyrimidin-2-yl)piperidine-4-carbonitrile;
1-(4-((2-(trifluoromethyl)benzyl)amino)pyrido[2,3-d]pyrimidin-2-yl)piperidine-3-carbonitrile;
2-(3-(methylsulfonyl)pyrrolidin-1-yl)-N-((2-(trifluoromethyl)pyridin-3-yl)methyl)pyrido[2,3-d]pyrimidin-4-amine;
N-((2-(trifluoromethyl)pyridin-3-yl)methyl)-2-(2-(trifluoromethyl) pyrrolidin-1-yl)pyrido[2,3-d]pyrimidin-4-amine;
2-(3-chloropyrrolidin-1-yl)-N-((2-(trifluoromethyl)pyridin-3-yl)methyl)pyrido[2,3-d]pyrimidin-4-amine;
N-(2-(trifluoromethyl)benzyl)-2-(3-(trifluoromethyl) pyrrolidin-1-yl)pyrido[2,3-d]pyrimidin-4-amine;
2-(2-oxa-5-azabicyclo[2.2.1]heptan-5-yl)-N-(2-(trifluoromethyl)benzyl)pyrido[2,3-d]pyrimidin-4-amine;
2-((6S)-2,6-dimethylmorpholino)-N-(2-(trifluoromethyl)benzyl)pyrido[2,3-d]pyrimidin-4-amine;
2-(3-isopropylmorpholino)-N-((2-(trifluoromethyl)pyridin-3-yl)methyl)pyrido[2,3-d]pyrimidin-4-amine;
2-((2R,3R)-2,3-dimethylmorpholino)-N-((2-(trifluoromethyl)pyridin-3-yl)methyl)pyrido[2,3-d]pyrimidin-4-amine;
2-((2S,5R)-2,5-dimethylmorpholino)-N-((2-(trifluoromethyl)pyridin-3-yl)methyl)pyrido[2,3-d]pyrimidin-4-amine;
2-(6-oxa-9-azaspiro[4.5]decan-9-yl)-N-((2-(trifluoromethyl)pyridin-3-yl)methyl)pyrido[2,3-d]pyrimidin-4-amine;
2-(4-(trifluoromethoxy)piperidin-1-yl)-N-((2-(trifluoromethyl)pyridin-3-yl)methyl)pyrido[2,3-d]pyrimidin-4-amine;
2-(4-(trifluoromethoxy)piperidin-1-yl)-N-((2-(trifluoromethyl)pyridin-3-yl)methyl)pyrido[2,3-d]pyrimidin-4-amine methanesulfonate;
2-(3-(difluoromethyl)azetidin-1-yl)-N-((2-(trifluoromethyl)pyridin-3-yl)methyl)pyrido[2,3-d]pyrimidin-4-amine;
2-((3R)-3,5-dimethylmorpholino)-N-((2-(trifluoromethyl)pyridin-3-yl)methyl)pyrido[2,3-d]pyrimidin-4-amine;
2-(3-(difluoromethyl)azetidin-1-yl)-N-(2-(trifluoromethyl)benzyl)pyrido[2,3-d]pyrimidin-4-amine;
2-((3R)-3,5-dimethylmorpholino)-N-(2-(trifluoromethyl)benzyl)pyrido[2,3-d]pyrimidin-4-amine;
2-(2-cyclopropylmorpholino)-N-((2-(trifluoromethyl)pyridin-3-yl)methyl)pyrido[2,3-d]pyrimidin-4-amine;
2-((2S,5S)-2,5-dimethylmorpholino)-N-((2-(trifluoromethyl)pyridin-3-yl)methyl)pyrido[2,3-d]pyrimidin-4-amine;
(R)-2-(3-(difluoromethoxy)pyrrolidin-1-yl)-N-((2-(trifluoromethyl)pyridin-3-yl)methyl)pyrido[2,3-d]pyrimidin-4-amine;
(S)-2-(3-(difluoromethoxy)pyrrolidin-1-yl)-N-((2-(trifluoromethyl)pyridin-3-yl)methyl)pyrido[2,3-d]pyrimidin-4-amine;
2-(2-(difluoromethyl)morpholino)-N-((2-(trifluoromethyl)pyridin-3-yl)methyl)pyrido[2,3-d]pyrimidin-4-amine;
4-(4-((2-(trifluoromethyl)benzyl)amino)pyrido[2,3-d]pyrimidin-2-yl)morpholine-2-carbonitrile;
2-(2-(difluoromethyl)morpholino)-N-(2-(trifluoromethyl)benzyl)pyrido[2,3-d]pyrimidin-4-amine;
2-(8-oxa-5-azaspiro[3.5]nonan-5-yl)-N-(2-(trifluoromethyl)benzyl)pyrido[2,3-d]pyrimidin-4-amine;
2-(9-oxa-6-azaspiro[4.5]decan-6-yl)-N-(2-(trifluoromethyl)benzyl)pyrido[2,3-d]pyrimidin-4-amine;
2-((2R,3S)-2,3-dimethylmorpholino)-N-((2-(trifluoromethyl)pyridin-3-yl)methyl)pyrido[2,3-d]pyrimidin-4-amine;
(3R)-3-methyl-4-(4-(2-(2-(trifluoromethyl)pyridin-3-yl)azetidin-1-yl)pyrido[2,3-d]pyrimidin-2-yl)morpholine;
(3S)-3-methyl-4-(4-(2-(2-(trifluoromethyl)pyridin-3-yl)azetidin-1-yl)pyrido[2,3-d]pyrimidin-2-yl)morpholine;
(3R)-3-methyl-4-(4-(2-(2-(trifluoromethyl)phenyl)azetidin-1-yl)pyrido[2,3-d]pyrimidin-2-yl)morpholine;
(3S)-3-methyl-4-(4-(2-(2-(trifluoromethyl)phenyl)azetidin-1-yl)pyrido[2,3-d]pyrimidin-2-yl)morpholine;
2-(3-(difluoromethoxy)piperidin-1-yl)-N-((2-(trifluoromethyl)pyridin-3-yl)methyl)pyrido[2,3-d]pyrimidin-4-amine;
2-(6-oxa-3-azabicyclo[3.1.1]heptan-3-yl)-N-((2-(trifluoromethyl)pyridin-3-yl)methyl)pyrido[2,3-d]pyrimidin-4-amine;
2-(2-oxa-5-azabicyclo[4.1.0]heptan-5-yl)-N-((2-(trifluoromethyl)pyridin-3-yl)methyl)pyrido[2,3-d]pyrimidin-4-amine;
2-(2,2,6,6-tetrafluoromorpholino)-N-((2-(trifluoromethyl)pyridin-3-yl)methyl)pyrido[2,3-d]pyrimidin-4-amine;
2-(4-azaspiro[2.5]octan-4-yl)-N-((2-(trifluoromethyl)pyridin-3-yl)methyl)pyrido[2,3-d]pyrimidin-4-amine;
2-(3-(trifluoromethoxy)pyrrolidin-1-yl)-N-((2-(trifluoromethyl)pyridin-3-yl)methyl)pyrido[2,3-d]pyrimidin-4-amine;
2-(5-azaspiro[3.4]octan-5-yl)-N-((2-(trifluoromethyl)pyridin-3-yl)methyl)pyrido[2,3-d]pyrimidin-4-amine;
2-(2-((trifluoromethoxy)methyl)pyrrolidin-1-yl)-N-((2-(trifluoromethyl)pyridin-3-yl)methyl)pyrido[2,3-d]pyrimidin-4-amine;
2-(6-oxa-3-azabicyclo[3.1.1]heptan-3-yl)-N-(2-(trifluoromethyl)benzyl)pyrido[2,3-d]pyrimidin-4-amine;
2-(2-oxa-5-azabicyclo[4.1.0]heptan-5-yl)-N-(2-(trifluoromethyl)benzyl)pyrido[2,3-d]pyrimidin-4-amine;
2-((3R)-3,5-dimethylmorpholino)-N-(2-(trifluoromethyl)benzyl)pyrido[2,3-d]pyrimidin-4-amine;
6-fluoro-2-(3-methylmorpholino)-N-((2-(trifluoromethyl)pyridin-3-yl)methyl)pyrido[2,3-d]pyrimidin-4-amine;
6-methoxy-2-(3-methylmorpholino)-N-((2-(trifluoromethyl)pyridin-3-yl)methyl)pyrido[2,3-d]pyrimidin-4-amine;
7-methoxy-2-(3-methylmorpholino)-N-((2-(trifluoromethyl)pyridin-3-yl)methyl)pyrido[2,3-d]pyrimidin-4-amine;
5-methoxy-2-(3-methylmorpholino)-N-((2-(trifluoromethyl)pyridin-3-yl)methyl)pyrido[2,3-d]pyrimidin-4-amine;
6,7-dimethoxy-2-(3-methylmorpholino)-N-((2-(trifluoromethyl)pyridin-3-yl)methyl)pyrido[2,3-d]pyrimidin-4-amine; and
2-(3-methylmorpholino)-7-(2-morpholinoethoxy)-N-((2-(trifluoromethyl)pyridin-3-yl)methyl)pyrido[2,3-d]pyrimidin-4-amine.

67. A compound for use/a method according to paragraph 65 or paragraph 66, wherein the compound of formula I is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine 2-(4-(trifluoromethoxy)piperidin-1-yl)-N-((2-(trifluoromethyl)pyridin-3-yl)methyl)pyrido[2,3-d]pyrimidin-4-amine, 2-((2S,5S)-2,5-dimethylmorpholino)-N-((2-(trifluoromethyl)pyridin-3-yl)methyl)pyrido[2,3-d]pyrimidin-4-amine and N²-isopropyl-N²-methyl-N⁴-((2-(trifluoromethyl)pyridin-3-yl)methyl)pyrido[2,3-d]pyrimidine-2,4-diamine or a pharmaceutically acceptable salt or solvate thereof.

68. A compound for use/a method according to paragraph 65 or paragraph 66, wherein the compound of formula I is selected from the group consisting of 2-(3-methylmorpholino)-N-((2-(trifluoromethyl)pyridin-3-yl)methyl)pyrido[2,3-d]pyrimidin-4-amine, 2-(4,4-difluoropiperidin-1-yl)-N-(2-(trifluoromethyl)benzyl)pyrido[2,3-d]pyrimidin-4-amine and 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof.

69. A compound for use/a method according to paragraph 65 or paragraph 66, wherein the compound of formula I is selected from the group consisting of 2-(4,4-difluoropiperidin-1-yl)-N-(2-(trifluoromethyl)benzyl)pyrido[2,3-d]pyrimidin-4-amine, or a pharmaceutically acceptable salt or solvate thereof.

70. A compound for use/a method according to paragraph 65 or paragraph 66, wherein the compound of formula I is selected from the group consisting of 2-(4-(trifluoromethoxy)piperidin-1-yl)-N-((2-(trifluoromethyl)pyridin-3-yl)methyl)pyrido[2,3-d]pyrimidin-4-amine, or a pharmaceutically acceptable salt or solvate thereof.

71. A compound for use/a method according to paragraph 65 or paragraph 66, wherein the compound of formula I is 2-(3-methylmorpholino)-N-((2-(trifluoromethyl)pyridin-3-yl)methyl)pyrido[2,3-d]pyrimidin-4-amine, or a pharmaceutically acceptable salt or solvate thereof.

72. A compound for use/a method according to paragraph 65 or paragraph 66, wherein the compound of formula I is (S)-2-(3-methylmorpholino)-N-((2-(trifluoromethyl)pyridin-3-yl)methyl)pyrido[2,3-d]pyrimidin-4-amine or a pharmaceutically acceptable salt or solvate thereof.

73. A compound for use/a method according to any one of the preceding paragraphs, wherein the compound of formula I is for use/used in combination with an anthelmintic agent.

74. A compound for use/a method according to paragraph 69, wherein the anthelmintic agent is selected from an a cestodal agent or a nematode agent.

75. A compound of formula I, or a pharmaceutically acceptable salt or solvate thereof, for use according to paragraph 69, wherein the anthelmintic agent is selected from a macrocyclic lactone; a benzenedisulfonamide compound; a benzimidazole compounds.

76. A compound for use/a method according to any one of paragraphs 1 to 68, wherein the compound of formula I is for use/used in combination with another anti-filarial agent.

77. A compound for use/a method according to paragraph 72, wherein the anti-filarial agent is selected from a macrocyclic lactone, a tetracycline antibiotic and a benzimidazole agent.

Though the present invention may relate to any compound or particular group of compounds for use in the methods defined herein by way of optional, preferred or suitable features or otherwise in terms of particular embodiments, the present invention may also relate to any compound or particular group of compounds that specifically excludes said optional, preferred or suitable features or particular embodiments.

### Salts and Solvates

The compounds of formula I described herein may be and used per se in the methods disclosed herein or may be used in the form of a pharmaceutically acceptable salt.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in mammals.

In one embodiment, the compounds of Formula I and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I and sub-formulae thereof may exist in solvated as well as unsolvated forms such as, for example, hydrated forms. It is to be understood that the invention encompasses all such solvated forms that possess the biological activity described herein.

### Routes of Administration

The compounds of the invention may be administered to a mammal by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In one embodiment, the compounds described herein can be used in the methods herein as oral formulations, injectable formulations, and topical, pour-on, dermal or subdermal formulations. The formulations are intended to be administered to a mammal, such as a human, a cat or a dog.

Compounds used in the methods described may be for oral use. For example, as baits, dietary supplements, troches, lozenges, chewables, tablets, hard or soft capsules, bolus, emulsions, aqueous or oily suspensions, aqueous or oily solutions, oral drench formulations, dispersible powders or granules, premixes, syrups or elixirs, enteric formulations or pastes.

Compounds used in the methods described may be for parenteral use. For instance, in the form of a sterile injectable aqueous or oleagenous suspension.

Compounds used in the methods described may be for topical, dermal and subdermal formulations. For instance, in the form of emulsions, creams, ointments, gels, pastes, powders, shampoos, pour-on formulations, ready-to-use formulations, spot-on solutions and suspensions, dips and sprays.

In one embodiment, the compound of formula I, or a pharmaceutically acceptable salt or solvate thereof is for administration orally. In another embodiment of the methods described herein the compound of formula I, or a pharmaceutically acceptable salt or solvate thereof is administered orally.

In one embodiment, the compound of formula I, or a pharmaceutically acceptable salt or solvate thereof is for administration orally. In another embodiment of the methods described herein the compound of formula I, or a pharmaceutically acceptable salt or solvate thereof is administered topically.

### Combinations

The compounds for use in the methods disclosed herein may be for use in combination with another anthelmintic agent.

Anthelmintic agents are well known in the art and include, but are not limited to, macrocyclic lactone anthelmintic compounds, such as the avermectin and milbemycin series of compounds; a benzenedisulfonamide compound; a cestodal agent; a benzimidazole compound.

The compounds for use in the methods disclosed herein may be for use in combination with another anti-filarial agent, such as a macrocyclic lactone, a tetracycline antibiotic and a benzimidazole agent.

### EXAMPLES

The following examples are provided solely to illustrate the present invention and are not intended to limit the scope of the invention, as described herein.

The compounds of formula I and salts and solvates thereof may be prepared using synthetic techniques that are known in the art. Exemplary methods of preparation are disclosed in PCT publication WO 2018/134685 (filed 17 Jan. 2018), the teachings of which are incorporated herein by reference.

### Example 1—In Vitro D. immitis Microfilariae Assay

Dogs experimentally infected with Missouri isolate D. immitis were maintained at the Filariasis Research Reagent Resource Centre (FR3, University of Georgia). Dog blood infected with D. immitis mf (Dimf) were shipped at ambient temperature to Liverpool, UK. Dimf were purified from dog blood by red blood cell lysis (0.1% ammonium chloride) and removal of white blood cells and dead mf by size exclusion chromatography (PD-10 sepharose columns, Amersham). Following buffer exchange by centrifugation, Dimf were placed into 96-well culture plates at a density of 8000 mf/well in 100 μl Dulbecco's Modified Eagles Medium (DMEM) supplemented with 10% foetal calf serum and antibiotics: 1% penicillin, streptomycin and amphotericin B (complete DMEM). Test compounds (doxycycline (DOX), Compound A and Compound B (see Table A, below) were prepared at 10 mM stocks in dimethyl sulphoxide (DMSO). Two-fold final dilutions in complete DMEM were added at 100 μl to five replicate wells containing Dimf. 1% DMSO in complete DMEM was used as a negative control. Mf assay plates were incubated for 6 days at 37° C./5% CO₂.

TABLE A
Compounds A to D

| | | |
|---|---|---|
| Compound A | 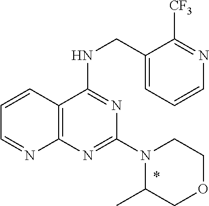 [SMILES: CC1COCCN1c1nc(NCc2cccnc2C(F)(F)F)c2cccnc2n1] | (S)-2-(3-methylmorpholino)-N-((2- (trifluoromethyl)pyridin-3- yl)methyl)pyrido[2,3-d]pyrimidin-4-amine |
| Compound B | 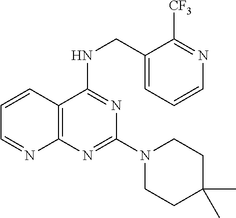 [SMILES: CC1(C)CCN(c2nc(NCc3cccnc3C(F)(F)F)c3cccnc3n2)CC1] | 2-(4,4-dimethylpiperidin-1-yl)-N-((2- (trifluoromethyl)pyridin-3- yl)methyl)pyrido[2,3-d]pyrimidin-4-amine |
| Compound C | 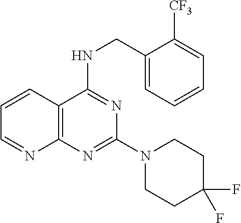 [SMILES: FC1(F)CCN(c2nc(NCc3ccccc3C(F)(F)F)c3cccnc3n2)CC1] | 2-(4,4-difluoropiperidin-1-yl)-N-(2- (trifluoromethyl)benzyl)pyrido[2,3- d]pyrimidin-4-amine |
| Compound D | 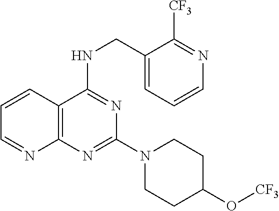 [SMILES: FC(F)(F)OC1CCN(c2nc(NCc3cccnc3C(F)(F)F)c3cccnc3n2)CC1] | 2-(4-(trifluoromethoxy)piperidin-1-yl)-N-((2- (trifluoromethyl)pyridin-3- yl)methyl)pyrido[2,3-d]pyrimidin-4-amine |

After six-day continuous exposures to drug, Dimf were harvested from plates, genomic DNA extracted using the QIAmp DNA Mini Kit (Qiagen) before undertaking quantitative (Q)PCR (Bio-Rad) to obtain an estimate of Wolbachia Surface Protein (wsp) gene copy number as a proxy for Wolbachia titre. Species-specific wsp primers were used. Enumerations of wsp copies were interpolated from a standard titration curve of known DNA content run on the same assay.

Table 1 illustrates the efficacy of doxycycline, Compound A or Compound B against D. immitis mf calculated as:

$$\% efficacy = 1 - \{ \frac{median wsp titre test group}{median wsp titre vehicle group} \} \times 100$$

TABLE 1
Efficacy of compounds of formula I against D. immitis mf in vitro

| | D. immitis mf |
|---|---|
| Drug | Efficacy |
| (dose) | (median % reduction Wolbachia) |
| Doxycycline | 58.8% |
| (5000 nM) | |
| Doxycycline | 10.0% |
| (50 nM) | |
| Compound A | 96.0% |
| (5000 nM) | |
| Compound A | 88.8% |
| (200 nM) | |
| Compound A | 64.1% |
| (50 nM) | |
| Compound B | 53.6% |
| (50 nM) | |
| Compound B | 63.9% |
| (0.5 nM) | |

This assay demonstrates D. immitis Wolbachia are susceptible to the azaquinazolines compounds of formula I such as Compound A and B.

### Example 2—In Vitro D. immitis L4 Larvae Assay

Heparinised dog blood containing D. immitis Missouri isolate (sourced from FR3 as described above) were adjusted to a density of 5000 mf/ml by mixing with uninfected blood before feeding to Aedes aegypti mosquitoes (Liverpool filarial susceptible strain) through an artificial membrane feeder (Hemotek®). Blood-fed mosquitoes were reared for 15 days to allow for development to infectious-stage L3. The L3 were collected from infected mosquitoes by crushing and concentration using a Baermann's apparatus and RPMI medium. Viable, motile DiL3 were enumerated into batches of 200 and loaded into 1 ml syringes with 25-gauge needles.

Male NOD. Cg-Prkdc^{scid} Il2rg^{tm1Wjl}/SzJ mice (NSG mice) at 5-6 weeks of age were purchased from Charles River, UK. Mice were inoculated with 200 DiL3 subcutaneously in the left flank. Two weeks following infection, mice were euthanised by rising CO₂ concentration and 14-day old fourth-stage D. immitis (DiL4) were retrieved from subcutaneous tissues.

Individual motile DiL4 were placed in to 96-well culture plates in 100 μl Dulbecco's Modified Eagles Medium (DMEM) supplemented with 10% foetal calf serum and antibiotics: 1% penicillin, streptomycin and amphotericin B (complete DMEM). Test compounds (doxycycline and Compound B) were prepared at 10 mM stocks in dimethyl sulphoxide (DMSO). Two-fold final dilutions in complete DMEM were added at 100 μl to between three and five replicate wells containing DiL4. 1% DMSO in complete DMEM was used as a negative control. Assay plates were incubated for 6 days at 37° C./5% CO₂.

After six-day continuous exposures to drug, DiL4 were removed from plates, genomic DNA extracted using the QIAmp DNA Mini Kit (Qiagen) before undertaking quantitative (Q)PCR (Bio-Rad) to obtain an estimate of D. immitis Wolbachia Surface Protein (wsp) gene copy number as a proxy for Wolbachia titre. Enumerations of wsp copies were interpolated from a standard titration curve of known DNA content run on the same assay.

Table 2 illustrates the efficacy of doxycycline or Compound B against D. immitis L4 calculated as:

$$\% efficacy = 1 - \{ \frac{median wsp titre test group}{median wsp titre vehicle group} \} \times 100$$

TABLE 2
Efficacy of azaquinazoline compounds against D. immitis L4 in vitro

| | D. immitis L4 |
|---|---|
| Drug | Efficacy |
| (dose) | (median % reduction Wolbachia) |
| Doxycycline | 65.9% |
| (5000 nM) | |
| Compound B | 57.4% |
| (50 nM) | |
| Compound B | 50.7% |
| (10 nM) | |
| Compound B | 45.4% |
| (1 nM) | |

The D. immitis L4 assay demonstrates D. immitis Wolbachia are susceptible to the azaquinazoline Compound B at low <100 nM dose exposures and doses between 50-10 nM mediate >50% depletions. This provides proof-of-concept that low concentrations of compound of formula I can deplete Wolbachia to a similar degree as the gold-standard, doxycycline, within developing stage Dirofilaria (the target life-cycle stage of a preventative chemotherapy strategy against heartworm).

### Example 3A—In Vivo D. immitis Larvae Mouse Assay

Heparinised dog blood containing D. immitis Missouri isolate (sourced from FR3 as described above) were adjusted to a density of 5000 mf/ml by mixing with uninfected blood before feeding to Aedes aegypti mosquitoes (Liverpool filarial susceptible strain) through an artificial membrane feeder (Hemotek®). Blood-fed mosquitoes were reared for 15 days to allow for development to infectious-stage L3. The L3 were collected from infected mosquitoes by crushing and concentration using a Baermann's apparatus and RPMI medium. Viable, motile DiL3 were enumerated into batches of 200 and loaded into 1 ml syringes with 25-gauge needles.

Male NOD. Cg-Prkdcscid Il2rgtm1Wjl/SzJ mice (NSG mice) at 5-6 weeks of age were purchased from Charles River, UK. Mice (n=3-4 per group) were inoculated with 200 DiL3 subcutaneously in the left flank. At point of infection mice commenced oral dosing with 50 mg/kg doxycycline monohydrate (Sigma Aldrich) once daily, 200 mg/kg Compound A twice-daily or vehicle control. Doxycycline was administered days 1-7 post-infection (seven day dosing), Compound A was administered days 1-2 (two day dosing). Doxycycline was prepared in distilled water, Compound A in PEG300/propylene glycol/H2O (55/25/20).

Two weeks following infection, mice were euthanised by rising CO₂ concentration and 14-day old fourth-stage D. immitis (DiL4) were retrieved from subcutaneous tissues.

Individual motile DiL4 (n=10-12 per treatment group) were separated and genomic DNA extracted using the QIAmp DNA Mini Kit (Qiagen) before undertaking quantitative (Q)PCR (Bio-Rad) to obtain an estimate of D. immitis Wolbachia Surface Protein (wsp) gene copy number as a proxy for Wolbachia titre. Enumerations of wsp copies were interpolated from a standard titration curve of known DNA content run on the same assay.

TABLE 3A
Efficacy of azaquinazoline compounds against D. immitis larvae in vivo

| | D. immitis L4 | |
|---|---|---|
| Drug | Efficacy (median % | n larvae |
| (dose) | reduction Wolbachia) | depleted ≥80% |
| Doxycycline | 69.6% | 4/9 |
| (50 mpk qd × 7) | | (44.4%) |
| Compound A | 89.7% | 10/11 |
| (200 mpk bid × 2) | | (90.9%) |

In conclusion, a short-course, 2-day regimen of Compound A mediates substantial, 89.7% median anti-Wolbachia activity in vivo against the D. immitis L3-L4 stage. This level of depletion is superior in terms of consistently mediating depletion >80% compared to 7-day dosing of doxycycline.

### Example 3B—In Vivo D. immitis Larvae Mouse Assay

Heparinised dog blood containing D. immitis Missouri isolate (sourced from FR3 as described above) were adjusted to a density of 5000 mf/ml by mixing with uninfected blood before feeding to Aedes aegypti mosquitoes (Liverpool filarial susceptible strain) through an artificial membrane feeder (Hemotek®). Blood-fed mosquitoes were reared for 15 days to allow for development to infectious-stage L3. The L3 were collected from infected mosquitoes by crushing and concentration using a Baermann's apparatus and RPMI medium. Viable, motile DiL3 were enumerated into batches of 200 and loaded into 1 ml syringes with 25-gauge needles.

Male NOD. Cg-Prkdc^{scid} Il2rg^{tm1Wjl}/SzJ mice (NSG mice) at 5-6 weeks of age were purchased from Charles River, UK. Mice (n=1-2 per group) were inoculated with 200 DiL3 subcutaneously in the left flank. At point of infection mice commenced oral dosing with 200 mg/kg Compound A, 200 mg/kg Compound C, or 200 mg/kg Compound D twice daily (see Table A, above) or were untreated. Dosing was given for one day (single day dosing). Compounds were prepared in PEG300/propylene glycol/H₂O (55/25/20).

Two weeks following infection, mice were euthanised by rising CO₂ concentration and 14-day old fourth-stage D. immitis (DiL4) were retrieved from subcutaneous tissues.

Individual motile DiL4 (n=3-12 per treatment group) were separated and genomic DNA extracted using the QIAmp DNA Mini Kit (Qiagen) before undertaking quantitative (Q)PCR (Bio-Rad) to obtain an estimate of D. immitis Wolbachia Surface Protein (wsp) gene copy number as a proxy for Wolbachia titre. Enumerations of wsp copies were interpolated from a standard titration curve of known DNA content run on the same assay.

Table 3B illustrates the anti-Wolbachia efficacy against D. immitis L3-L4 in NSG mice, calculated as:

$$\% efficacy = 1 - \{ \frac{median wsp titre test group}{median wsp titre vehicle group} \} \times 100$$

TABLE 3B
Efficacy of compounds of formula I against
D. immitis larval Wolbachia in vivo

| | D. immitis L4 | |
|---|---|---|
| Drug | Efficacy | n larvae |
| (dose) | (median % reduction Wolbachia) | depleted ≥50% |
| Compound A | 52.1% | 4/5 |
| (200 mpk bid × 1) | | (80%) |
| Compound C | 61.7% | 2/3 |
| (200 mpk bid × 1) | | (67%) |
| Compound D | 53.6% | 8/12 |
| (200 mpk bid × 1) | | (67%) |

In conclusion, a 1-day regimen of Compound A mediates >50% median anti-Wolbachia activity in vivo against the D. immitis L3-L4 stage. This level of depletion or higher can be achieved also with analogues such as Compound C and Compound D.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

### REFERENCES

Taylor, M. J., Hoerauf, A., Bockarie, M., 2010. Lymphatic filariasis and onchocerciasis. Lancet 376, 1175-1185.
Simon, F., Siles-Lucas, M., Morchon, R., Gonzalez-Miguel, J., Mellado, I., Carreton, E., Montoya-Alonso, J. A., 2012. Human and animal dirofilariasis: the emergence of a zoonotic mosaic. Clin. Microbiol. Rev. 25, 507-544.
McCall, J. W., Genchi, C., Kramer, L. H., Guerrero, J., Venco, L., 2008. Heartworm disease in animals and humans. Adv. Parasitol. 66, 193-285.
Genchi, C., Kramer, L., 2017. Subcutaneous dirofilariosis (Dirofilaria repens): an infection spreading throughout the old world. Parasit. Vectors 10, 517.
Lee, A. C., Montgomery, S. P., Theis, J. H., Blagburn, B. L., Eberhard, M. L., 2010. Public health issues concerning the widespread distribution of canine heartworm disease. Trends Parasitol. 26, 168-173.
AHS, 2016. Heartworm by the Numbers. https://d3ft8sckhnqim2.cloudfront.net/images/pdf/2019HeartwormByTheNumbers.pdf?1548859511.
Bowman, D., Little, S. E., Lorentzen, L., Shields, J., Sullivan, M. P., Carlin, E. P., 2009. Prevalence and geographic distribution of Dirofilaria immitis, Borrelia burgdorferi, Ehrlichia canis, and Anaplasma phagocytophilum in dogs in the United States: results of a national clinic-based serologic survey. Vet. Parasitol. 160, 138-148.
Morchon, R., Carreton, E., Gonzalez-Miguel, J., Mellado-Hernandez, I., 2012. Heartworm disease (Dirofilaria immitis) and their vectors in Europe—new distribution trends. Front. Physiol. 3, 196.
Mallawarachchi, C. H., Chandrasena, N., Wickramasinghe, S., Premaratna, R., Gunawardane, N., Mallawarachchi, N., de Silva, N. R., 2018a. A preliminary survey of filarial parasites in dogs and cats in Sri Lanka. PLoS One 13, e0206633.
Khowawisetsut, L., Sarasombath, P. T., Thammapalo, S., Loymek, S., Korbarsa, T., Nochote, H., Phuakrod, A., Choochote, W., Wongkamchai, S., 2017. Therapeutic trial of doxycyclin plus ivermectin for the treatment of Brugia malayi naturally infected cats. Vet. Parasitol. 245, 42-47.
Mallawarachchi, C. H., Nilmini Chandrasena, T. G. A., Premaratna, R., Mallawarachchi, S., de Silva, N. R., 2018b. Human infection with sub-periodic Brugia spp. in Gampaha District, Sri Lanka: a threat to filariasis elimination status? Parasit. Vectors 11, 68.
Prichard, R. K., Geary, T. G., 2019. Perspectives on the utility of moxidectin for the control of parasitic nematodes in the face of developing anthelmintic resistance. Int. J. Parasitol. Drugs Drug Resist. 10, 69-83.
Wolstenholme, A. J., Evans, C. C., Jimenez, P. D., Moorhead, A. R., 2015. The emergence of macrocyclic lactone resistance in the canine heartworm, Dirofilaria immitis. Parasitology 142, 1249-1259.
Clark, J. N., Daurio, C. P., Plue, R. E., Wallace, D. H., Longhofer, S. L., 1992. Efficacy of ivermectin and pyrantel pamoate combined in a chewable formulation against heartworm, hookworm, and ascarid infections in dogs. Am. J. Vet. Res. 53, 517-520.
Di Cesare, A., Braun, G., Di Giulio, E., Paoletti, B., Aquilino, V., Bartolini, R., La Torre, F., Meloni, S., Drake, J., Pandolfi, F., Avolio, S., Traversa, D., 2014. Field clinical study evaluating the efficacy and safety of an oral formulation containing milbemycin oxime/praziquantel (Milbemax®, Novartis Animal Health) in the chemoprevention of the zoonotic canine infection by Dirofilaria repens. Parasit. Vectors 7, 347.
Kryda, K., Six, R. H., Walsh, K. F., Holzmer, S. J., Chapin, S., Mahabir, S. P., Myers, M., Inskeep, T., Rugg, J., Cundiff, B., Pullins, A., Ulrich, M., McCall, J. W., McTier, T. L., Maeder, S. J., 2019. Laboratory and field studies to investigate the efficacy of a novel, orally administered combination product containing moxidectin, sarolaner and pyrantel for the prevention of heartworm disease (Dirofilaria immitis) in dogs. Parasit. Vectors 12, 445.
Pollono, F., Pollmeier, M., Rossi, L., 1998. The prevention of Dirofilaria repens infection with ivermectin/pyrantel chewables. Parassitologia 40, 457-459.
McCall, J. W., McTier, T. L., Ryan, W. G., Gross, S. J., Soll, M. D., 1996. Evaluation of ivermectin and milbemycin oxime efficacy against Dirofilaria immitis infections of three and four months' duration in dogs. Am. J. Vet. Res. 57, 1189-1192.
Paul, A. J., Todd Jr, K. S., Sundberg, J. P., DiPietro, J. A., McCall, J. W., 1986. Efficacy of ivermectin against Dirofilaria immitis larvae in dogs 30 and 45 days after induced infection. Am. J. Vet. Res. 47, 883-884.
McTier, T. L., Kryda, K., Wachowski, M., Mahabir, S., Ramsey, D., Rugg, D., Mazaleski, M., Therrien, C., Adams, E., Wolff, T., Bowman, D. D., 2019. ProHeart® 12, a moxidectin extended-release injectable formulation for prevention of heartworm (Dirofilaria immitis) disease in dogs in the USA for 12 months. Parasit. Vectors 12, 369.
Bourguinat, C., Keller, K., Bhan, A., Peregrine, A., Geary, T., Prichard, R., 2011a. Macrocyclic lactone resistance in Dirofilaria immitis. Vet. Parasitol. 181, 388-392.
Pulaski, C. N., Malone, J. B., Bourguinat, C., Prichard, R., Geary, T., Ward, D., Klei, T. R., Guidry, T., Smith, G., Delcambre, B., Bova, J., Pepping, J., Carmichael, J., Schenker, R., Pariaut, R., 2014. Establishment of macrocyclic lactone resistant Dirofilaria immitis isolates in experimentally infected laboratory dogs. Parasit. Vectors 7, 494.
Snyder, D. E., Wiseman, S., Bowman, D. D., McCall, J. W., Reinemeyer, C. R., 2011. Assessment of the effectiveness of a combination product of spinosad and milbemycin oxime on the prophylaxis of canine heartworm infection. Vet. Parasitol. 180, 262-266.
Bourguinat, C., Lee, A. C., Lizundia, R., Blagburn, B. L., Liotta, J. L., Kraus, M. S., Keller, K., Epe, C., Letourneau, L., Kleinman, C. L., Paterson, T., Gomez, E. C., Montoya-Alonso, J. A., Smith, H., Bhan, A., Peregrine, A. S., Carmichael, J., Drake, J., Schenker, R., Kaminsky, R., Bowman, D. D., Geary, T. G., Prichard, R. K., 2015. Macrocyclic lactone resistance in Dirofilaria immitis: failure of heartworm preventives and investigation of genetic markers for resistance. Vet. Parasitol. 210, 167-178.
Kramer, L., Grandi, G., Passeri, B., Gianelli, P., Genchi, M., Dzimianski, M. T., Supakorndej, P., Mansour, A. M., Supakorndej, N., McCall, S. D., McCall, J. W., 2011. Evaluation of lung pathology in Dirofilaria immitis-experimentally infected dogs treated with doxycycline or a combination of doxycycline and ivermectin before administration of melarsomine dihydrochloride. Vet. Parasitol. 176, 357-360.
AHS, 2019a. Current Canine Guidelines for the Prevention, Diagnosis and Management of Heartworm (Dirofilaria immitis) in Dogs. https://d3ft8sckhnqim2.cloudfront.net/images/pdf/2018_AHS_Canine_Guidelines_rev_7-25-19.pdf?1564157216.
AHS, 2019b. Current Feline Guidelines for the Prevention, Diagnosis and Management of Heartworm (Dirofilaria immitis) Infection in Cats. https://d3ft8sckhnqim2.cloudfront.net/images/pdf/Feline-Guidelines-Summary.pdf?1393457997.
Bosshardt, S. C. et al. Prophylactic activity of tetracycline against Brugia pahangi infection in jirds (Meriones unguiculatus). J Parasitol 79, 775-777 (1993).

1. A compound of formula I, or pharmaceutically acceptable salt or solvate thereof, for use in the treatment or prevention of a Dirofilaria infection in a mammal, 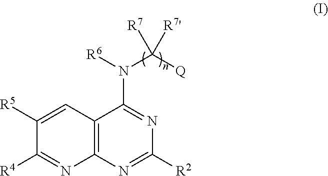
wherein
Q is a group selected from an C₃₋₁₁cycloalkyl optionally substituted by one or more R^{b}, 3-15 membered heterocycloalkyl optionally substituted by one or more R^{b}, C₆₋₁₁ aryl group optionally substituted with by one or more R^{b}, 5-15 membered heteroaryl optionally substituted by one or more R^{b};
R⁶ is selected from hydrogen and C₁₋₆ alkyl;
R⁷ and R^{7′} are independently selected from hydrogen, C₃₋₆ cycloalkyl, C₁₋₆ alkyl, where said C₃₋₆ cycloalkyl and C₁₋₆ alkyl are optionally substituted by one or more R^{a}; or
R⁷ and R^{7′}, together with the carbon to which they are attached form a 3-7 membered cycloalkyl ring, optionally substituted with one or more R^{a}, or R⁷ and R^{7′}, together with the carbon to which they are attached form a carbonyl group; or
R⁶ and R⁷, together with the atoms to which they are attached form a 3-7 membered heterocyclic ring, optionally substituted with one or more R^{a};
n is a number selected from 1, 2 and 3;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1}, —OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1}, C₁₋₁₀ haloalkyl, C₁₋₁₀alkyl optionally substituted by one or more R^{e}, C₂₋₆alkenyl optionally substituted by one or more R^{e}, C₂₋₆alkynyl optionally substituted by one or more R^{e}, C₆₋₁₁aryl optionally substituted by one or more R^{e}, (C₇₋₁₆)alkylaryl optionally substituted by one or more R^{e}, C₃₋₁₁cycloalkyl optionally substituted by one or more R^{e}, (C₄₋₁₇)cycloalkylalkyl optionally substituted by one or more R^{e}, 3-15 membered heterocycloalkyl optionally substituted by one or more R^{e}, 4-21 membered alkylheterocycloalkyl optionally substituted by one or more R^{e}, 5-15 membered heteroaryl optionally substituted by one or more R^{e}, and 6-21 membered alkylheteroaryl optionally substituted by one or more R^{e};
where each R^{c1} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d1} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c1} and R^{d1}, when attached to the same atom, together with the atom to which they are attached form a 3-7 membered ring, optionally containing one or more for heteroatoms selected from O, NH and S, and wherein said ring is optionally substituted with one or more R^{a};
where each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl and 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆alkyl;
each R^{b} and R^{e} is independently selected from hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl;
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, amino, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; or
R^{c2} and R^{d2}, when attached to the same atom, together with the atom to which they are attached form a 3-7 membered ring, optionally containing one or more for heteroatoms selected from O, NH and S, and wherein said ring is optionally substituted with one or more R^{a};
R³, R⁴ and R⁵ are independently selected from hydrogen, hydroxyl, halogen, CN, C₁₋₆ haloalkyl, C₁₋₆ haloalkoxy, C₁₋₆ alkyl, phenyl and cyclopropyl, wherein said C₁₋₆ alkyl, phenyl and cyclopropyl are optionally substituted by one or more R^{a};

2. A compound of formula I, or a pharmaceutically acceptable salt or solvate thereof, for use according to claim 1 wherein the Dirofilaria infection is infection with one or more parasite selected from D. immitis, D. repens and D. tenuis.

3. A compound of formula I, or a pharmaceutically acceptable salt or solvate thereof, for use according to claim 1 or claim 2 wherein the Dirofilaria infection is infection with D. immitis.

4. A compound of formula I, or a pharmaceutically acceptable salt or solvate thereof, for use according to any one of the preceding claims wherein the Dirofilaria infection is infection with Dirofilaria microfilariae, Dirofilaria third stage (L3) larvae, Dirofilaria fourth stage (L4) larvae, Dirofilaria fifth stage (L5) larvae, an adult dirofilarial worm or a combination thereof.

5. A compound of formula I, or a pharmaceutically acceptable salt or solvate thereof, for use according to any one of claims 1 to 3 wherein the Dirofilaria infection is infection with D. immitis microfilariae (mf), L3 larvae, L4 larvae or a combination thereof.

6. A compound of formula I, or a pharmaceutically acceptable salt or solvate thereof, for use according to any one of the preceding claims wherein the mammal is selected from a human, a dog, a cat, a racoon, a coyote, a jackal, a wolf, a fox, a ferret.

7. A compound of formula I, or a pharmaceutically acceptable salt or solvate thereof, for use according to any one of the preceding claims wherein the mammal is selected from a human, a dog and a cat.

8. A compound of formula I, or a pharmaceutically acceptable salt or solvate thereof, for use according to any one of the preceding claims wherein the mammal is a dog.

9. A compound of formula I, or a pharmaceutically acceptable salt or solvate thereof, as defined in claim 1 for use in the treatment or prevention of a disease or condition caused by a Dirofilaria infection in a mammal.

10. A compound of formula I, or a pharmaceutically acceptable salt or solvate thereof, for use according to claim 9 wherein the disease or condition is selected from heartworm disease (HWD), heartworm associated respiratory disease (HARD) or a dermatological condition.

11. A compound of formula I, or a pharmaceutically acceptable salt or solvate thereof, for use according to claim 9 or claim 10 wherein the disease or condition is selected from heartworm disease (HWD) or a dermatological condition and the mammal is a dog.

12. A compound of formula I, or a pharmaceutically acceptable salt or solvate thereof, for use according to claim 9 or 10 wherein the disease or condition is selected from heartworm associated respiratory disease (HARD) and the mammal is a cat.

13. A compound of formula I, or a pharmaceutically acceptable salt or solvate thereof, for use according to any one of claims 9 to 12 wherein the Dirofilaria infection is D. immitis infection.

14. A compound of formula I, or a pharmaceutically acceptable salt or solvate thereof, for use according to any one of the preceding claims wherein the compound is for administration for 7 consecutive days per month or less.

15. A compound of formula I, or a pharmaceutically acceptable salt or solvate thereof, for use according to any one of the preceding claims wherein the compound is for administration for 1 or 2 consecutive days per month or less.

16. A compound of formula I, or a pharmaceutically acceptable salt or solvate thereof, for use according to any one of the preceding claims wherein the compound is for oral administration.

17. A compound of formula I, or a pharmaceutically acceptable salt or solvate thereof, for use according to any one of the preceding claims, wherein the compound is of sub-formula Ia: 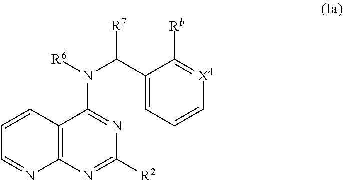
wherein,
R⁶ is selected from hydrogen and C₁₋₆ alkyl;
R⁷ is selected from hydrogen, ═O, C₃₋₆ cycloalkyl, C₁₋₆ alkyl, where said C₃₋₆ cycloalkyl, C₁₋₆ alkyl are optionally substituted by one or more R^{a}; or
R⁶ and R⁷, together with the atoms to which they are attached form a 3-7 membered heterocyclic ring, optionally substituted with one or more R^{a};
X⁴ is selected from CH and N;
R² is selected from —CN, —C(═O)R^{d1}, —C(═O)OR^{d1}, —C(═O)NR^{c1}R^{d1}, —C(O)C(═O)R^{d1}, —NR^{c1}R^{d1}, —NR^{c1}C(═O)R^{d1}, —NR^{c1}C(═O)OR^{d1}, —NR^{c1}C(═O)NR^{c1}R^{d1}, —NR^{c1}S(═O)₂R^{d1}, —NR^{c1}S(═O)₂NR^{c1}R^{d1}, —OR^{d1}, —SR^{d1}, —OC(═O)R^{d1}, —OC(═O)NR^{c1}R^{d1}, —OC(═O)OR^{d1}, —S(═O)R^{d1}, —S(═O)₂R^{d1}, —OS(═O)R^{d1}, —OS(═O)₂R^{d1}, —OS(═O)₂OR^{d1}, —S(═O)NR^{c1}R^{d1}, —OS(═O)₂NR^{c1}R^{d1}, —S(═O)₂NR^{c1}R^{d1}, C₁₋₁₀haloalkyl, C₁₋₁₀alkyl optionally substituted by 1-13 R^{e}, C₂₋₆alkenyl optionally substituted by 1-11 R^{e}, C₂₋₆alkynyl optionally substituted by 1-9 R^{e}, C₆₋₁₁aryl optionally substituted by 1-11 R^{e}, (C₇₋₁₆)alkylaryl optionally substituted by 1-9 R^{e}, C₃₋₁₁cycloalkyl optionally substituted by 1-21 R^{e}, (C₄₋₁₇)cycloalkylalkyl optionally substituted by 1-32 R^{e}, 3-15 membered heterocycloalkyl optionally substituted by 1-28 R^{e}, 4-21 membered alkylheterocycloalkyl optionally substituted by 1-40 R^{e}, 5-15 membered heteroaryl optionally substituted by 1-15 R^{e}, and 6-21 membered alkylheteroaryl optionally substituted by 1-27 R^{e};
each R^{a} is independently selected from hydroxyl, halogen, CN, C₁₋₆ haloalkyl, C₁₋₆ haloalkoxy, C₁₋₆ alkyl, C₃₋₆ cycloalkyl, 3-7 membered heterocycloalkyl, wherein said C₃₋₆ cycloalkyl, 3-7 membered heterocycloalkyl are optionally substituted with one or more groups selected from hydroxyl, halogen, CN, C₁₋₆ haloalkyl, C₃₋₆ cycloalkyl, C₁₋₆ alkyl and O—C₁₋₆ alkyl;
each R^{b} and R^{e} is independently selected from hydroxyl, ═O, halogen, CN, C₁₋₆ haloalkyl, C₁₋₆ haloalkoxy, C₃₋₆ cycloalkyl, C₁₋₆ alkyl, O—C₁₋₆ alkyl, C₃₋₆ cycloalkyl, 3-7 membered heterocycloalkyl, —C(═O)R^{d2}, —C(═O)OR^{d2}, —C(═O)NR^{c2}R^{d2}, —C(O)C(═O)R^{d2}, —NR^{c2}R^{d2}, —NR^{c2}C(═O)R^{d2}, —NR^{c2}C(═O)OR^{d2}, —NR^{c2}C(═O)NR^{c2}R^{d2}, —NR^{c2}S(═O)₂R^{d2}, NR^{c2}S(═O)₂NR^{c2}R^{d2}, —OR^{d2}, —SR^{d2}, —OC(═O)R^{d2}, —OC(═O)NR^{c2}R^{d2}, —OC(═O)OR^{d2}, —S(═O)₂R^{d2}, —S(═O)R^{d2}, —OS(═O)R^{d2}, —OS(═O)₂R^{d2}, —OS(═O)₂OR^{d2}, —S(═O)NR^{c2}R^{d2}, —OS(═O)₂NR^{c2}R^{d2}, and —S(═O)₂NR^{c2}R^{d2}, where said C₃₋₆ cycloalkyl, C₁₋₆ alkyl, and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
each R^{c2} is independently selected from hydrogen, hydroxyl, halogen, CN, C₁₋₆ haloalkyl, C₃₋₆ cycloalkyl, C₁₋₆ alkyl and O—C₁₋₆ alkyl; and
each R^{d2} is independently selected from hydrogen, hydroxyl, halogen, CN, C₁₋₆ haloalkyl, 3-7 membered heterocycloalkyl, C₃₋₆ cycloalkyl, C₁₋₆ alkyl and O—C₁₋₆ alkyl, C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, halogen, CN, C₁₋₆ haloalkyl, C₃₋₆ cycloalkyl, C₆₋₁₁ aryl, C₁₋₆ alkyl and O—C₁₋₆ alkyl; or
R^{c2} and R^{d2}, when attached to the same atom, together with the atom to which they are attached form a 3-7 membered ring, optionally substituted with one or more R^{a}.

18. A compound of formula I, or a pharmaceutically acceptable salt or solvate thereof, for use according to any one of the preceding claims, wherein the compound of formula I is selected from:
N²-isopropyl-N²,N⁴-dimethyl-N⁴-((2-(trifluoromethyl)pyridin-3-yl)methyl)pyrido[2,3-d]pyrimidine-2,4-diamine;
N²-isopropyl-N²-methyl-N⁴-((2-(trifluoromethyl)pyridin-3-yl)methyl)pyrido[2,3-d]pyrimidine-2,4-diamine;
N²-isopropyl-N²-methyl-N⁴-((2-(trifluoromethyl)pyridin-3-yl)methyl)pyrido[2,3-d]pyrimidine-2,4-diamine methanesulfonate;
2-(azetidin-1-yl)-N-((2-(trifluoromethyl)pyridin-3-yl)methyl)pyrido[2,3-d]pyrimidin-4-amine;
N⁴-(2-fluoro-6-(trifluoromethyl)benzyl)-N², N²-dimethylpyrido[2,3-d]pyrimidine-2,4-diamine;
3-methyl-1-(4-(((2-(trifluoromethyl)pyridin-3-yl)methyl)amino)pyrido[2,3-d]pyrimidin-2-yl)azetidin-3-ol;
2-(2-methylazetidin-1-yl)-N-((2-(trifluoromethyl)pyridin-3-yl)methyl)pyrido[2,3-d]pyrimidin-4-amine;
2-(2,2-dimethylazetidin-1-yl)-N-((2-(trifluoromethyl)pyridin-3-yl)methyl)pyrido[2,3-d]pyrimidin-4-amine;
2-(pyrrolidin-1-yl)-N-((2-(trifluoromethyl)pyridin-3-yl)methyl)pyrido[2,3-d]pyrimidin-4-amine;
N²-cyclopropyl-N²-methyl-N⁴-((2-(trifluoromethyl)pyridin-3-yl)methyl)pyrido[2,3-d]pyrimidine-2,4-diamine;
N-isopropyl-N-methyl-4-(2-(2-(trifluoromethyl)phenyl)azetidin-1-yl)pyrido[2,3-d]pyrimidin-2-amine;
N-isopropyl-N-methyl-4-(2-(2-(trifluoromethyl)phenyl)piperidin-1-yl)pyrido[2,3-d]pyrimidin-2-amine;
N-isopropyl-N-methyl-4-(3-(2-(trifluoromethyl)phenyl)morpholino)pyrido[2,3-d]pyrimidin-2-amine;
N-isopropyl-N-methyl-4-(2-(2-(trifluoromethyl)phenyl)pyrazolidin-1-yl)pyrido[2,3-d]pyrimidin-2-amine;
2-morpholino-N-((2-(trifluoromethyl)pyridin-3-yl)methyl)pyrido[2,3-d]pyrimidin-4-amine;
2-(3-methylmorpholino)-N-((2-(trifluoromethyl)pyridin-3-yl)methyl)pyrido[2,3-d]pyrimidin-4-amine;
2-(3-methylmorpholino)-N-((2-(trifluoromethyl)pyridin-3-yl)methyl)pyrido[2,3-d]pyrimidin-4-amine methanesulfonate;
2-(3,3-difluoropyrrolidin-1-yl)-N-((2-(trifluoromethyl)pyridin-3-yl)methyl)pyrido[2,3-d]pyrimidin-4-amine;
2-(3,3-difluoropyrrolidin-1-yl)-N-((2-(trifluoromethyl)pyridin-3-yl)methyl)pyrido[2,3-d]pyrimidin-4-amine methanesulfonate;
2-(4-fluoropiperidin-1-yl)-N-((2-(trifluoromethyl)pyridin-3-yl)methyl)pyrido[2,3-d]pyrimidin-4-amine;
2-(4,4-difluoropiperidin-1-yl)-N-(2-(trifluoromethyl)benzyl)pyrido[2,3-d]pyrimidin-4-amine;
2-(4,4-difluoropiperidin-1-yl)-N-((2-(trifluoromethyl)pyridin-3-yl)methyl)pyrido[2,3-d]pyrimidin-4-amine;
2-(4-chloropiperidin-1-yl)-N-(2-(trifluoromethyl)benzyl)pyrido[2,3-d]pyrimidin-4-amine;
2-(4-chloropiperidin-1-yl)-N-((2-(trifluoromethyl)pyridin-3-yl)methyl)pyrido[2,3-d]pyrimidin-4-amine;
2-(3-fluoroazetidin-1-yl)-N-((2-(trifluoromethyl)pyridin-3-yl)methyl)pyrido[2,3-d]pyrimidin-4-amine;
2-(3,3-difluoroazetidin-1-yl)-N-((2-(trifluoromethyl)pyridin-3-yl)methyl)pyrido[2,3-d]pyrimidin-4-amine;
2-(3-(trifluoromethyl)azetidin-1-yl)-N-((2-(trifluoromethyl)pyridin-3-yl)methyl)pyrido[2,3-d]pyrimidin-4-amine;
(S)-2-(3-methylmorpholino)-N-(2-(trifluoromethyl)benzyl)pyrido[2,3-d]pyrimidin-4-amine;
(S)-2-(3-methylmorpholino)-N-(2-(trifluoromethyl)benzyl)pyrido[2,3-d]pyrimidin-4-amine methanesulfonate;
(R)-2-(3-methylmorpholino)-N-(2-(trifluoromethyl)benzyl)pyrido[2,3-d]pyrimidin-4-amine;
(S)-2-(3-methylmorpholino)-N-((2-(trifluoromethyl)pyridin-3-yl)methyl)pyrido[2,3-d]pyrimidin-4-amine;
(R)-2-(3-methylmorpholino)-N-((2-(trifluoromethyl)pyridin-3-yl)methyl)pyrido[2,3-d]pyrimidin-4-amine;
2-(4-chloropiperidin-1-yl)-4-(2-(2-(trifluoromethyl)phenyl)azetidin-1-yl)pyrido[2,3-d]pyrimidine;
2-(4,4-difluoropiperidin-1-yl)-4-(2-(2-(trifluoromethyl)phenyl)azetidin-1-yl)pyrido[2,3-d]pyrimidine;
3-methyl-4-(4-(2-(2-(trifluoromethyl)phenyl)azetidin-1-yl)pyrido[2,3-d]pyrimidin-2-yl)morpholine;
2-(3,3-difluoropyrrolidin-1-yl)-4-(2-(2-(trifluoromethyl)phenyl)azetidin-1-yl)pyrido[2,3-d]pyrimidine;
2-(3-(trifluoromethyl)azetidin-1-yl)-4-(2-(2-(trifluoromethyl)phenyl)azetidin-1-yl)pyrido[2,3-d]pyrimidine;
2-(4-chloropiperidin-1-yl)-4-(2-(2-(trifluoromethyl)pyridin-3-yl)azetidin-1-yl)pyrido[2,3-d]pyrimidine;
2-(4,4-difluoropiperidin-1-yl)-4-(2-(2-(trifluoromethyl)pyridin-3-yl)azetidin-1-yl)pyrido[2,3-d]pyrimidine;
3-methyl-4-(4-(2-(2-(trifluoromethyl)pyridin-3-yl)azetidin-1-yl)pyrido[2,3-d]pyrimidin-2-yl)morpholine;
2-(3,3-difluoropyrrolidin-1-yl)-4-(2-(2-(trifluoromethyl)pyridin-3-yl)azetidin-1-yl)pyrido[2,3-d]pyrimidine;
2-(3-(trifluoromethyl)azetidin-1-yl)-4-(2-(2-(trifluoromethyl)pyridin-3-yl)azetidin-1-yl)pyrido[2,3-d]pyrimidine;
2-((4-chloropiperidin-1-yl)methyl)-N-((2-(trifluoromethyl)pyridin-3-yl)methyl)pyrido[2,3-d]pyrimidin-4-amine;
2-((4,4-difluoropiperidin-1-yl)methyl)-N-((2-(trifluoromethyl)pyridin-3-yl)methyl)pyrido[2,3-d]pyrimidin-4-amine;
2-((3-methylmorpholino)methyl)-N-((2-(trifluoromethyl)pyridin-3-yl)methyl)pyrido[2,3-d]pyrimidin-4-amine;
2-((3,3-difluoropyrrolidin-1-yl)methyl)-N-((2-(trifluoromethyl)pyridin-3-yl)methyl)pyrido[2,3-d]pyrimidin-4-amine;
2-((3-(trifluoromethyl)azetidin-1-yl)methyl)-N-((2-(trifluoromethyl)pyridin-3-yl)methyl)pyrido[2,3-d]pyrimidin-4-amine;
2-((4-chloropiperidin-1-yl)methyl)-N-((2-(trifluoromethyl)pyridin-3-yl)methyl)pyrido[2,3-d]pyrimidin-4-amine;
2-((4,4-difluoropiperidin-1-yl)methyl)-N-((2-(trifluoromethyl)pyridin-3-yl)methyl)pyrido[2,3-d]pyrimidin-4-amine;
2-((3-methylmorpholino)methyl)-N-((2-(trifluoromethyl)pyridin-3-yl)methyl)pyrido[2,3-d]pyrimidin-4-amine;
2-((3,3-difluoropyrrolidin-1-yl)methyl)-N-((2-(trifluoromethyl)pyridin-3-yl)methyl)pyrido[2,3-d]pyrimidin-4-amine;
2-((3-(trifluoromethyl)azetidin-1-yl)methyl)-N-((2-(trifluoromethyl)pyridin-3-yl)methyl)pyrido[2,3-d]pyrimidin-4-amine;
2-(4-(methylsulfonyl)piperidin-1-yl)-N-((2-(trifluoromethyl)pyridin-3-yl)methyl)pyrido[2,3-d]pyrimidin-4-amine;
2-(4,4-dimethylpiperidin-1-yl)-N-((2-(trifluoromethyl)pyridin-3-yl)methyl)pyrido[2,3-d]pyrimidin-4-amine;
1-(4-(((2-(trifluoromethyl)pyridin-3-yl)methyl)amino)pyrido[2,3-d]pyrimidin-2-yl)piperidine-4-carbonitrile;
2-(4-(trifluoromethyl)piperidin-1-yl)-N-((2-(trifluoromethyl)pyridin-3-yl)methyl)pyrido[2,3-d]pyrimidin-4-amine;
N-((2-(trifluoromethyl)pyridin-3-yl)methyl)-2-(3-(trifluoromethyl) pyrrolidin-1-yl)pyrido[2,3-d]pyrimidin-4-amine;
1-(4-(((2-(trifluoromethyl)pyridin-3-yl)methyl)amino)pyrido[2,3-d]pyrimidin-2-yl)azetidine-3-carbonitrile;
1-(4-(((2-(trifluoromethyl)pyridin-3-yl)methyl)amino)pyrido[2,3-d]pyrimidin-2-yl)pyrrolidine-2-carbonitrile;
2-(2,2-dimethylmorpholino)-N-((2-(trifluoromethyl)pyridin-3-yl)methyl)pyrido[2,3-d]pyrimidin-4-amine;
2-(3,3-dimethylmorpholino)-N-((2-(trifluoromethyl)pyridin-3-yl)methyl)pyrido[2,3-d]pyrimidin-4-amine;
2-(2-methylmorpholino)-N-((2-(trifluoromethyl)pyridin-3-yl)methyl)pyrido[2,3-d]pyrimidin-4-amine;
2-(3-fluoropyrrolidin-1-yl)-N-((2-(trifluoromethyl)pyridin-3-yl)methyl)pyrido[2,3-d]pyrimidin-4-amine;
2-(4-(methylsulfonyl)piperidin-1-yl)-N-(2-(trifluoromethyl)benzyl)pyrido[2,3-d]pyrimidin-4-amine;
2-(4,4-dimethylpiperidin-1-yl)-N-(2-(trifluoromethyl)benzyl)pyrido[2,3-d]pyrimidin-4-amine;
1-(4-((2-(trifluoromethyl)benzyl)amino)pyrido[2,3-d]pyrimidin-2-yl)azetidine-3-carbonitrile;
2-(3,3-dimethylmorpholino)-N-(2-(trifluoromethyl)benzyl)pyrido[2,3-d]pyrimidin-4-amine;
2-(2,2-dimethylmorpholino)-N-(2-(trifluoromethyl)benzyl)pyrido[2,3-d]pyrimidin-4-amine;
2-(2-methylmorpholino)-N-(2-(trifluoromethyl)benzyl)pyrido[2,3-d]pyrimidin-4-amine;
2-(3-(trifluoromethyl)piperidin-1-yl)-N-((2-(trifluoromethyl)pyridin-3-yl)methyl)pyrido[2,3-d]pyrimidin-4-amine;
4-methyl-1-(4-(((2-(trifluoromethyl)pyridin-3-yl)methyl)amino)pyrido[2,3-d]pyrimidin-2-yl)piperidine-4-carbonitrile;
1-(4-(((2-(trifluoromethyl)pyridin-3-yl)methyl)amino)pyrido[2,3-d]pyrimidin-2-yl)piperidine-3-carbonitrile;
2-(2,2-difluoro-7-azaspiro[3.5]nonan-7-yl)-N-((2-(trifluoromethyl)pyridin-3-yl)methyl)pyrido[2,3-d]pyrimidin-4-amine;
2-(2,2-difluoro-7-azaspiro[3.5]nonan-7-yl)-N-((2-(trifluoromethyl)pyridin-3-yl)methyl)pyrido[2,3-d]pyrimidin-4-amine methanesulfonate;
2-(3-cyclopropylmorpholino)-N-((2-(trifluoromethyl)pyridin-3-yl)methyl)pyrido[2,3-d]pyrimidin-4-amine;
2-((6S)-2,6-dimethylmorpholino)-N-((2-(trifluoromethyl)pyridin-3-yl)methyl)pyrido[2,3-d]pyrimidin-4-amine;
2-(2-oxa-5-azabicyclo[2.2.1]heptan-5-yl)-N-((2-(trifluoromethyl)pyridin-3-yl)methyl)pyrido[2,3-d]pyrimidin-4-amine;
2-(4-oxa-7-azaspiro[2.5]octan-7-yl)-N-((2-(trifluoromethyl)pyridin-3-yl)methyl)pyrido[2,3-d]pyrimidin-4-amine;
4-(4-(((2-(trifluoromethyl)pyridin-3-yl)methyl)amino)pyrido[2,3-d]pyrimidin-2-yl)morpholine-2-carbonitrile;
2-(3-(fluoromethyl)piperidin-1-yl)-N-((2-(trifluoromethyl)pyridin-3-yl)methyl)pyrido[2,3-d]pyrimidin-4-amine;
2-(2-(trifluoromethyl)piperidin-1-yl)-N-((2-(trifluoromethyl)pyridin-3-yl)methyl)pyrido[2,3-d]pyrimidin-4-amine;
2-(2,2-difluoro-7-azaspiro[3.5]nonan-7-yl)-N-(2-(trifluoromethyl)benzyl)pyrido[2,3-d]pyrimidin-4-amine;
4-methyl-1-(4-((2-(trifluoromethyl)benzyl)amino)pyrido[2,3-d]pyrimidin-2-yl)piperidine-4-carbonitrile;
1-(4-((2-(trifluoromethyl)benzyl)amino)pyrido[2,3-d]pyrimidin-2-yl)piperidine-3-carbonitrile;
2-(3-(methylsulfonyl)pyrrolidin-1-yl)-N-((2-(trifluoromethyl)pyridin-3-yl)methyl)pyrido[2,3-d]pyrimidin-4-amine;
N-((2-(trifluoromethyl)pyridin-3-yl)methyl)-2-(2-(trifluoromethyl)pyrrolidin-1-yl)pyrido[2,3-d]pyrimidin-4-amine;
2-(3-chloropyrrolidin-1-yl)-N-((2-(trifluoromethyl)pyridin-3-yl)methyl)pyrido[2,3-d]pyrimidin-4-amine;
N-(2-(trifluoromethyl)benzyl)-2-(3-(trifluoromethyl)pyrrolidin-1-yl)pyrido[2,3-d]pyrimidin-4-amine;
2-(2-oxa-5-azabicyclo[2.2.1]heptan-5-yl)-N-(2-(trifluoromethyl)benzyl)pyrido[2,3-d]pyrimidin-4-amine;
2-((6S)-2,6-dimethylmorpholino)-N-(2-(trifluoromethyl)benzyl)pyrido[2,3-d]pyrimidin-4-amine;
2-(3-isopropylmorpholino)-N-((2-(trifluoromethyl)pyridin-3-yl)methyl)pyrido[2,3-d]pyrimidin-4-amine;
2-((2R,3R)-2,3-dimethylmorpholino)-N-((2-(trifluoromethyl)pyridin-3-yl)methyl)pyrido[2,3-d]pyrimidin-4-amine;
2-((2S,5R)-2,5-dimethylmorpholino)-N-((2-(trifluoromethyl)pyridin-3-yl)methyl)pyrido[2,3-d]pyrimidin-4-amine;
2-(6-oxa-9-azaspiro[4.5]decan-9-yl)-N-((2-(trifluoromethyl)pyridin-3-yl)methyl)pyrido[2,3-d]pyrimidin-4-amine;
2-(4-(trifluoromethoxy)piperidin-1-yl)-N-((2-(trifluoromethyl)pyridin-3-yl)methyl)pyrido[2,3-d]pyrimidin-4-amine;
2-(4-(trifluoromethoxy)piperidin-1-yl)-N-((2-(trifluoromethyl)pyridin-3-yl)methyl)pyrido[2,3-d]pyrimidin-4-amine methanesulfonate;
2-(3-(difluoromethyl)azetidin-1-yl)-N-((2-(trifluoromethyl)pyridin-3-yl)methyl)pyrido[2,3-d]pyrimidin-4-amine;
2-((3R)-3,5-dimethylmorpholino)-N-((2-(trifluoromethyl)pyridin-3-yl)methyl)pyrido[2,3-d]pyrimidin-4-amine;
2-(3-(difluoromethyl)azetidin-1-yl)-N-(2-(trifluoromethyl)benzyl)pyrido[2,3-d]pyrimidin-4-amine;
2-((3R)-3,5-dimethylmorpholino)-N-(2-(trifluoromethyl)benzyl)pyrido[2,3-d]pyrimidin-4-amine;
2-(2-cyclopropylmorpholino)-N-((2-(trifluoromethyl)pyridin-3-yl)methyl)pyrido[2,3-d]pyrimidin-4-amine;
2-((2S,5S)-2,5-dimethylmorpholino)-N-((2-(trifluoromethyl)pyridin-3-yl)methyl)pyrido[2,3-d]pyrimidin-4-amine;
(R)-2-(3-(difluoromethoxy)pyrrolidin-1-yl)-N-((2-(trifluoromethyl)pyridin-3-yl)methyl)pyrido[2,3-d]pyrimidin-4-amine;
(S)-2-(3-(difluoromethoxy)pyrrolidin-1-yl)-N-((2-(trifluoromethyl)pyridin-3-yl)methyl)pyrido[2,3-d]pyrimidin-4-amine;
2-(2-(difluoromethyl)morpholino)-N-((2-(trifluoromethyl)pyridin-3-yl)methyl)pyrido[2,3-d]pyrimidin-4-amine;
4-(4-((2-(trifluoromethyl)benzyl)amino)pyrido[2,3-d]pyrimidin-2-yl)morpholine-2-carbonitrile;
2-(2-(difluoromethyl)morpholino)-N-(2-(trifluoromethyl)benzyl)pyrido[2,3-d]pyrimidin-4-amine;
2-(8-oxa-5-azaspiro[3.5]nonan-5-yl)-N-(2-(trifluoromethyl)benzyl)pyrido[2,3-d]pyrimidin-4-amine;
2-(9-oxa-6-azaspiro[4.5]decan-6-yl)-N-(2-(trifluoromethyl)benzyl)pyrido[2,3-d]pyrimidin-4-amine;
2-((2R,3S)-2,3-dimethylmorpholino)-N-((2-(trifluoromethyl)pyridin-3-yl)methyl)pyrido[2,3-d]pyrimidin-4-amine;
(3R)-3-methyl-4-(4-(2-(2-(trifluoromethyl)pyridin-3-yl)azetidin-1-yl)pyrido[2,3-d]pyrimidin-2-yl)morpholine;
(3S)-3-methyl-4-(4-(2-(2-(trifluoromethyl)pyridin-3-yl)azetidin-1-yl)pyrido[2,3-d]pyrimidin-2-yl)morpholine;
(3R)-3-methyl-4-(4-(2-(2-(trifluoromethyl)phenyl)azetidin-1-yl)pyrido[2,3-d]pyrimidin-2-yl)morpholine;
(3S)-3-methyl-4-(4-(2-(2-(trifluoromethyl)phenyl)azetidin-1-yl)pyrido[2,3-d]pyrimidin-2-yl)morpholine;
2-(3-(difluoromethoxy)piperidin-1-yl)-N-((2-(trifluoromethyl)pyridin-3-yl)methyl)pyrido[2,3-d]pyrimidin-4-amine;
2-(6-oxa-3-azabicyclo[3.1.1]heptan-3-yl)-N-((2-(trifluoromethyl)pyridin-3-yl)methyl)pyrido[2,3-d]pyrimidin-4-amine;
2-(2-oxa-5-azabicyclo[4.1.0]heptan-5-yl)-N-((2-(trifluoromethyl)pyridin-3-yl)methyl)pyrido[2,3-d]pyrimidin-4-amine;
2-(2,2,6,6-tetrafluoromorpholino)-N-((2-(trifluoromethyl)pyridin-3-yl)methyl)pyrido[2,3-d]pyrimidin-4-amine;
2-(4-azaspiro[2.5]octan-4-yl)-N-((2-(trifluoromethyl)pyridin-3-yl)methyl)pyrido[2,3-d]pyrimidin-4-amine;
2-(3-(trifluoromethoxy)pyrrolidin-1-yl)-N-((2-(trifluoromethyl)pyridin-3-yl)methyl)pyrido[2,3-d]pyrimidin-4-amine;
2-(5-azaspiro[3.4]octan-5-yl)-N-((2-(trifluoromethyl)pyridin-3-yl)methyl)pyrido[2,3-d]pyrimidin-4-amine;
2-(2-((trifluoromethoxy)methyl)pyrrolidin-1-yl)-N-((2-(trifluoromethyl)pyridin-3-yl)methyl)pyrido[2,3-d]pyrimidin-4-amine;
2-(6-oxa-3-azabicyclo[3.1.1]heptan-3-yl)-N-(2-(trifluoromethyl)benzyl)pyrido[2,3-d]pyrimidin-4-amine;
2-(2-oxa-5-azabicyclo[4.1.0]heptan-5-yl)-N-(2-(trifluoromethyl)benzyl)pyrido[2,3-d]pyrimidin-4-amine;
2-((3R)-3,5-dimethylmorpholino)-N-(2-(trifluoromethyl)benzyl)pyrido[2,3-d]pyrimidin-4-amine;
6-fluoro-2-(3-methylmorpholino)-N-((2-(trifluoromethyl)pyridin-3-yl)methyl)pyrido[2,3-d]pyrimidin-4-amine;
6-methoxy-2-(3-methylmorpholino)-N-((2-(trifluoromethyl)pyridin-3-yl)methyl)pyrido[2,3-d]pyrimidin-4-amine;
7-methoxy-2-(3-methylmorpholino)-N-((2-(trifluoromethyl)pyridin-3-yl)methyl)pyrido[2,3-d]pyrimidin-4-amine;
5-methoxy-2-(3-methylmorpholino)-N-((2-(trifluoromethyl)pyridin-3-yl)methyl)pyrido[2,3-d]pyrimidin-4-amine;
6,7-dimethoxy-2-(3-methylmorpholino)-N-((2-(trifluoromethyl)pyridin-3-yl)methyl)pyrido[2,3-d]pyrimidin-4-amine; and
2-(3-methylmorpholino)-7-(2-morpholinoethoxy)-N-((2-(trifluoromethyl)pyridin-3-yl)methyl)pyrido[2,3-d]pyrimidin-4-amine.

19. A compound of formula I, or a pharmaceutically acceptable salt or solvate thereof, for use according to any one of the preceding claims, wherein the compound of formula I is 2-(3-methylmorpholino)-N-((2-(trifluoromethyl)pyridin-3-yl)methyl)pyrido[2,3-d]pyrimidin-4-amine, or (S)-2-(3-methylmorpholino)-N-((2-(trifluoromethyl)pyridin-3-yl)methyl)pyrido[2,3-d]pyrimidin-4-amine.

20. A compound of formula I, or a pharmaceutically acceptable salt or solvate thereof, for use according to any one of the preceding claims, wherein the compound is for use in combination with another anti-filarial agent.
